SEQUENCE LISTING (56 sequences)

SEQ ID NO: 1 - DNA, 789 bp, Homo sapiens
  source 1..789: mol_type = genomic DNA
atgaggaactcctatagatttctggcatcctctctctcagttgtcgtttctctcctgctaattcctgaagatgtctgtgaaaaaattattggaggaaatgaagtaactcctcattcaagaccctacatggtcctacttagtcttgacagaaaaaccatctgtgctggggctttgattgcaaaagactgggtgttgactgcagctcactgtaacttgaacaaaaggtcccaggtcattcttggggctcactcaataaccagggaagagccaacaaaacagataatgcttgttaagaaagagtttccctatccatgctatgacccagccacacgcgaaggtgaccttaaacttttacagctgacggaaaaagcaaaaattaacaaatatgtgactatccttcatctacctaaaaagggggatgatgtgaaaccaggaaccatgtgccaagttgcagggtggggcaggactcacaatagtgcatcttggtccgatactctgagagaagtcaatatcaccatcatagacagaaaagtctgcaatgatcgaaatcactataattttaaccctgtgattggaatgaatatggtttgtgctggaagcctccgaggtggaagagactcgtgcaatggagattctggaagccctttgttgtgcgagggtgttttccgaggggtcacttcctttggccttgaaaataaatgcggagaccctcgtgggcctggtgtctatattcttctctcaaagaaacacctcaactggataattatgactatcaagggagcagtttaa

SEQ ID NO: 2 - DNA, 90 bp, Homo sapiens
  source 1..90: mol_type = genomic DNA
atgaggaactcctatagatttctggcatcctctctctcagttgtcgtttctctcctgctaattcctgaagatgtctgtgaaaaaattatt

SEQ ID NO: 3 - DNA, 740 bp, Homo sapiens
  source 1..740: mol_type = genomic DNA
atgcagccaatactgctcctgttggccttcctcctcctcccgcgggcagatgcaggcgaaataattggtgggcatgaagcgaaaccccactcacgcccatatatggcttatttgatgatatgggaccagaaatctcttaaacgatgcggtggatttctcatccgcgatgattttgtgctcactgcagctcattgttgggggtcaagtatcaatgtaacactcggtgctcacaatatcaaagagcaagagcccacgcaacagtttatacccgttaaacggccgatccctcatcctgcctataaccccaagaacttctctaacgacattatgctccttcaattggagcggaaggctaagcgaacaagagcggttcagcccctccggctgccttccaacaaagctcaagtaaaaccgggccagacgtgctccgtcgcagggtggggtcaaactgccccactcggcaagcactcccacacgcttcaggaagtgaagatgacagtgcaggaagaccgcaagtgcgaaagcgaccttcggcactattacgactctacaatcgaattgtgcgtgggagatcctgagatcaagaagacctccttcaagggggatagtgggggtccactggtttgcaacaaggtagctcagggtatagtgtcttatggacgcaataacgggatgccccctagagcttgcaccaaagtgtcatccttcgccattggattaaaaagacaatgaagcggtac

SEQ ID NO: 4 - DNA, 66 bp, Homo sapiens
  source 1..66: mol_type = genomic DNA
atgcagccaatactgctcctgttggccttcctcctcctcccgcgggcagatgcaggcgaaataatt

SEQ ID NO: 5 - DNA, 741 bp, Homo sapiens
  source 1..741: mol_type = genomic DNA
atgcagccattcctcctcctgttggcctttcttctgacccctggggctgggacagaggagatcatcgggggccatgaggccaagccccactcccgcccctacatggcctttgttcagtttctgcaagagaagagtcggaagaggtgtggcggcatcctagtgagaaaggactttgtgctgacagctgctcactgccagggaagctccataaatgtcaccttgggggcccacaatatcaaggaacaggagcggacccagcagtttatccctgtgaaaagacccatcccccatccagcctataatcctaagaacttctccaacgacatcatgctactgcagctggagagaaaggccaagtggaccacagctgtgcggcctctcaggctacctagcagcaaggcccaggtgaagccagggcagctgtgcagtgtggctggctggggttatgtctcaatgagcactttagcaaccacactgcaggaagtgttgctgacagtgcagaaggactgccagtgtgaacgtctcttccatggcaattacagcagagccactgagatttgtgtgggggatccaaagaagacacagaccggtttcaagggggactccggggggcccctcgtgtgtaaggacgtagcccaaggtattctctcctatggaaataaaaaagggacacctccaggagtctacatcaaggtctcacacttcctgccctggataaagagaacaatgaagcgcctctaa

SEQ ID NO: 6 - DNA, 66 bp, Homo sapiens
  source 1..66: mol_type = genomic DNA
atgcagccattcctcctcctgttggcctttcttctgacccctggggctgggacagaggagatcatc

SEQ ID NO: 7 - DNA, 795 bp, Homo sapiens
  source 1..795: mol_type = genomic DNA
atgactaagttttcttccttttctctgtttttcctaatagttggggcttatatgactcatgtgtgtttcaatatggaaattattggagggaaagaagtgtcacctcattccaggccatttatggcctccatccagtatggcggacatcacgtttgtggaggtgttctgattgatccacagtgggtgctgacagcagcccactgccaatatcggtttaccaaaggccagtctcccactgtggttttaggcgcacactctctctcaaagaatgaggcctccaaacaaacactggagatcaaaaaatttataccattctcaagagttacatcagatcctcaatcaaatgatatcatgctggttaagcttcaaacagccgcaaaactcaataaacatgtcaagatgctccacataagatccaaaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaattaa

SEQ ID NO: 8 - DNA, 84 bp, Homo sapiens
  source 1..84: mol_type = genomic DNA
atgactaagttttcttccttttctctgtttttcctaatagttggggcttatatgactcatgtgtgtttcaatatggaaattatt

SEQ ID NO: 9 - DNA, 774 bp, Homo sapiens
  source 1..774: mol_type = genomic DNA
atggaggcctgcgtgtcttcactgctggtgctggccctgggggccctgtcagtaggcagctcctttgggacccagatcatcgggggccgggaggtgatcccccactcgcgcccgtacatggcctcactgcagagaaatggctcccacctgtgcgggggtgtcctggtgcacccaaagtgggtgctgacggctgcccactgcctggcccagcggatggcccagctgaggctggtgctggggctccacaccctggacagccccggtctcaccttccacatcaaggcagccatccagcaccctcgctacaagcccgtccctgccctggagaacgacctcgcgctgcttcagctggacgggaaagtgaagcccagccggaccatccggccgttggccctgcccagtaagcgccaggtggtggcagcagggactcggtgcagcatggccggctgggggctgacccaccagggcgggcgcctgtcccgggtgctgcgggagctggacctccaagtgctggacacccgcatgtgtaacaacagccgcttctggaacggcagcctctcccccagcatggtctgcctggcggccgactccaaggaccaggctccctgcaagggtgactcgggcgggcccctggtgtgtggcaaaggccgggtgttggccggagtcctgtccttcagctccagggtctgcactgacatcttcaagcctcccgtggccaccgctgtggcgccttacgtgtcctggatcaggaaggtcaccggccgatcggcctga

SEQ ID NO: 10 - DNA, 81 bp, Homo sapiens
  source 1..81: mol_type = genomic DNA
atggaggcctgcgtgtcttcactgctggtgctggccctgggggccctgtcagtaggcagctcctttgggacccagatcatc

SEQ ID NO: 11 - DNA, 438 bp, Homo sapiens
  source 1..438: mol_type = genomic DNA
atggctacctgggccctcctgctccttgcagccatgctcctgggcaacccaggtctggtcttctctcgtctgagccctgagtactacgacctggcaagagcccacctgcgtgatgaggagaaatcctgcccgtgcctggcccaggagggcccccagggtgacctgttgaccaaaacacaggagctgggccgtgactacaggacctgtctgacgatagtccaaaaactgaagaagatggtggataagcccacccagagaagtgtttccaatgctgcgacccgggtgtgtaggacggggaggtcacgatggcgcgacgtctgcagaaatttcatgaggaggtatcagtctagagttacccagggcctcgtggccggagaaactgcccagcagatctgtgaggacctcaggttgtgtataccttctacaggtcccctctga

SEQ ID NO: 12 - DNA, 66 bp, Homo sapiens
  source 1..66: mol_type = genomic DNA
atggctacctgggccctcctgctccttgcagccatgctcctgggcaacccaggtctggtcttctct

SEQ ID NO: 13 - DNA, 477 bp, Homo sapiens
  source 1..477: mol_type = genomic DNA
atgatgcagaagctactcaaatgcagtcggcttgtcctggctcttgccctcatcctggttctggaatcctcagttcaaggttatcctacgcagagagccaggtaccaatgggtgcgctgcaatccagacagtaattctgcaaactgccttgaagaaaaaggaccaatgttcgaactacttccaggtgaatccaacaagatcccccgtctgaggactgacctttttccaaagacgagaatccaggacttgaatcgtatcttcccactttctgaggactactctggatcaggcttcggctccggctccggctctggatcaggatctgggagtggcttcctaacggaaatggaacaggattaccaactagtagacgaaagtgatgctttccatgacaaccttaggtctcttgacaggaatctgccctcagacagccaggacttgggtcaacatggattagaagaggattttatgttataa

SEQ ID NO: 14 - DNA, 81 bp, Homo sapiens
  source 1..81: mol_type = genomic DNA
atgatgcagaagctactcaaatgcagtcggcttgtcctggctcttgccctcatcctggttctggaatcctcagttcaaggt

SEQ ID NO: 15 - DNA, 1668 bp, Homo sapiens
  source 1..1668: mol_type = genomic DNA
atggcagcccgtctgctcctcctgggcatccttctcctgctgctgcccctgcccgtccctgccccgtgccacacagccgcacgctcagagtgcaagcgcagccacaagttcgtgcctggtgcatggctggccggggagggtgtggacgtgaccagcctccgccgctcgggctccttcccagtggacacacaaaggttcctgcggcccgacggcacctgcaccctctgtgaaaatgccctacaggagggcaccctccagcgcctgcctctggcgctcaccaactggcgggcccagggctctggctgccagcgccatgtaaccagggccaaagtcagctccactgaagctgtggcccgggatgcggctcgtagcatccgcaacgactggaaggtcgggctggacgtgactcctaagcccaccagcaatgtgcatgtgtctgtggccggctcacactcacaggcagccaactttgcagcccagaagacccaccaggaccagtacagcttcagcactgacacggtggagtgccgcttctacagtttccatgtggtacacactcccccgctgcaccctgacttcaagagggccctcggggacctgccccaccacttcaacgcctccacccagcccgcctacctcaggcttatctccaactacggcacccacttcatccgggctgtggagctgggtggccgcatatcggccctcactgccctgcgcacctgcgagctggccctggaagggctcacggacaacgaggtggaggactgcctgactgtcgaggcccaggtcaacataggcatccacggcagcatctctgccgaagccaaggcctgtgaggagaagaagaagaagcacaagatgacggcctccttccaccaaacctaccgggagcgccactcggaagtggttggcggccatcacacctccattaacgacctgctgttcgggatccaggccgggcccgagcagtactcagcctgggtaaactccgtgcccggcagccctggcctggtggactacaccctggaacccctgcacgtgctgctggacagccaggacccgcggcgggaggcactgaggagggccctgagtcagtacctgacggacagggctcgctggagggactgcagccggccgtgcccaccagggcggcagaagagcccccgagacccatgccagtgtgtgtgccatggctcagcggtcaccacccaggactgctgccctcggcagaggggcctggcccagctggaggtgaccttcatccaagcatggagcctgtggggggactggttcactgccacggatgcctatgtgaagctcttctttggtggccaggagctgaggacgagcaccgtgtgggacaataacaaccccatctggtcagtgcggctggattttggggatgtgctcctggccacaggggggcccctgaggttgcaggtctgggatcaggactctggcagggacgatgacctccttggcacctgtgatcaggctcccaagtctggttcccatgaggtgagatgcaacctgaatcatggccacctaaaattccgctatcatgccaggtgcttgccccacctgggaggaggcacctgcctggactatgtcccccaaatgcttctgggggagcctccaggaaaccggagtggggccgtgtggtga

SEQ ID NO: 16 - DNA, 63 bp, Homo sapiens
  source 1..63: mol_type = genomic DNA
atggcagcccgtctgctcctcctgggcatccttctcctgctgctgcccctgcccgtccctgcc

SEQ ID NO: 17 - DNA, 172 bp, Homo sapiens
  source 1..172: mol_type = genomic DNA
acagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaagg

SEQ ID NO: 18 - DNA, 60 bp, Homo sapiens
  source 1..60: mol_type = genomic DNA
gaactggaagtggaatgtgccacacagctgcggagattcggcgacaagctgaacttccgg

SEQ ID NO: 19 - DNA, 60 bp, Homo sapiens
  source 1..60: mol_type = genomic DNA
tcgtccgccgcgcagctcaccgccgcccggctcaaggcgctaggcgacgagctgcaccag

SEQ ID NO: 20 - DNA, 60 bp, Homo sapiens
  source 1..60: mol_type = genomic DNA
ctctgggcagcacagcgctatggccgcgagctccggaggatgagtgacgagtttgtggac

SEQ ID NO: 21 - DNA, 60 bp, Homo sapiens
  source 1..60: mol_type = genomic DNA
gacatcatccggaatattgccaggcacctcgcccaggtcggggacagcatggaccgtagc

SEQ ID NO: 22 - DNA, 60 bp, Homo sapiens
  source 1..60: mol_type = genomic DNA
atgcgcccagagatatggatcgcccaagagttgcggcgtatcggagacgagtttaacgct

SEQ ID NO: 23 - DNA, 60 bp, Homo sapiens
  source 1..60: mol_type = genomic DNA
ttcaaagcctggggcaagttcggaggcgccttttgccggtatgcggatgagatgaaagaa

SEQ ID NO: 24 - DNA, 165 bp, Homo sapiens
  source 1..165: mol_type = genomic DNA
atgcctgggaagaaggcgcgcaagaacgctcaaccgagccccgcgcgggctccagcagagctggaagtcgagtgtgctactcaactcaggagatttggagacaaactgaacttccggcagaaacttctgaatctgatatccaaactcttctgctcaggaacctga

SEQ ID NO: 25 - DNA, 276 bp, Homo sapiens
  source 1..276: mol_type = genomic DNA
atgtgcccgtgccccctgcaccgcggccgcggccccccggccgtgtgcgcctgcagcgcgggtcgcctggggctgcgctcgtccgccgcgcagctcaccgccgcccggctcaaggcgctaggcgacgagctgcaccagcgcaccatgtggcggcgccgcgcgcggagccggagggcgccggcgcccggcgcgctccccacctactggccttggctgtgcgcggccgcgcaggtggcggcgctggcggcctggctgctcggcaggcggaacttgtag

SEQ ID NO: 26 - DNA, 507 bp, Homo sapiens
  source 1..507: mol_type = genomic DNA
atgttccagatcccagagtttgagccgagtgagcaggaagactccagctctgcagagaggggcctgggccccagccccgcaggggacgggccctcaggctccggcaagcatcatcgccaggccccaggcctcctgtgggacgccagtcaccagcaggagcagccaaccagcagcagccatcatggaggcgctggggctgtggagatccggagtcgccacagctcctaccccgcggggacggaggacgacgaagggatgggggaggagcccagcccctttcggggccgttcgcgctcggcgccccccaacctctgggcagcacagcgctatggccgcgagctccggaggatgagtgacgagtttgtggactcctttaagaagggacttcctcgcccgaagagcgcgggcacagcaacgcagatgcggcaaagctccagctggacgcgagtcttccagtcctggtgggatcggaacttgggcaggggaagctccgccccctcccagtga

SEQ ID NO: 27 - DNA, 588 bp, Homo sapiens
  source 1..588: mol_type = genomic DNA
atggactgtgaggtcaacaacggttccagcctcagggatgagtgcatcacaaacctactggtgtttggcttcctccaaagctgttctgacaacagcttccgcagagagctggacgcactgggccacgagctgccagtgctggctccccagtgggagggctacgatgagctgcagactgatggcaaccgcagcagccactcccgcttgggaagaatagaggcagattctgaaagtcaagaagacatcatccggaatattgccaggcacctcgcccaggtcggggacagcatggaccgtagcatccctccgggcctggtgaacggcctggccctgcagctcaggaacaccagccggtcggaggaggaccggaacagggacctggccactgccctggagcagctgctgcaggcctaccctagagacatggagaaggagaagaccatgctggtgctggccctgctgctggccaagaaggtggccagtcacacgccgtccttgctccgtgatgtctttcacacaacagtgaattttattaaccagaacctacgcacctacgtgaggagcttagccagaaatgggatggactga

SEQ ID NO: 28 - DNA, 597 bp, Homo sapiens
  source 1..597: mol_type = genomic DNA
atggcaaagcaaccttctgatgtaagttctgagtgtgaccgagaaggtagacaattgcagcctgcggagaggcctccccagctcagacctggggcccctacctccctacagacagagccacaaggtaatcctgaaggcaatcacggaggtgaaggggacagctgcccccacggcagccctcagggcccgctggccccacctgccagccctggcccttttgctaccagatccccgcttttcatctttatgagaagatcctccctgctgtctcgatcctccagtgggtatttctcttttgacacagacaggagcccagcacccatgagttgtgacaaatcaacacaaaccccaagtcctccttgccaggccttcaaccactatctcagtgcaatggcttccatgaggcaggctgaacctgcagatatgcgcccagagatatggatcgcccaagagttgcggcgtatcggagacgagtttaacgcttactatgcaaggagggtatttttgaataattaccaagcagccgaagaccacccacgaatggttatcttacgactgttacgttacattgtccgcctggtgtggagaatgcattga

SEQ ID NO: 29 - DNA, 483 bp, Homo sapiens
  source 1..483: mol_type = genomic DNA
atgtctgaagtaagacccctctccagagacatcttgatggagaccctcctgtatgagcagctcctggaacccccgaccatggaggttcttggcatgactgactctgaagaggacctggaccctatggaggacttcgattctttggaatgcatggagggcagtgacgcattggccctgcggctggcctgcatcggggacgagatggacgtgagcctcagggccccgcgcctggcccagctctccgaggtggccatgcacagcctgggtctggctttcatctacgaccagactgaggacatcagggatgttcttagaagtttcatggacggtttcaccacacttaaggagaacataatgaggttctggagatccccgaaccccgggtcctgggtgtcctgcgaacaggtgctgctggcgctgctgctgctgctggcgctgctgctgccgctgctcagcgggggcctgcacctgctgctcaagtga

SEQ ID NO: 30 - DNA, 582 bp, Homo sapiens
  source 1..582: mol_type = genomic DNA
atggcccgcgcacgccaggagggcagctccccggagcccgtagagggcctggcccgcgacggcccgcgccccttcccgctcggccgcctggtgccctcggcagtgtcctgcggcctctgcgagcccggcctggctgccgcccccgccgcccccaccctgctgcccgctgcctacctctgcgcccccaccgccccacccgccgtcaccgccgccctggggggttcccgctggcctgggggtccccgcagccggccccgaggcccgcgcccggacggtcctcagccctcgctctcgctggcggagcagcacctggagtcgcccgtgcccagcgccccgggggctctggcgggcggtcccacccaggcggccccgggagtccgcggggaggaggaacagtgggcccgggagatcggggcccagctgcggcggatggcggacgacctcaacgcacagtacgagcggcggagacaagaggagcagcagcggcaccgcccctcaccctggagggtcctgtacaatctcatcatgggactcctgcccttacccaggggccacagagcccccgagatggagcccaattag

SEQ ID NO: 31 - DNA, 555 bp, Homo sapiens
  source 1..555: mol_type = genomic DNA
atggagccatctcagtgtgtggaggagctggaggatgatgtgttccaaccagaggatggggagccggtgacccaacccgggagcttgctctctgctgacctgtttgcccagagcctactggactgccccctcagccgacttcagctcttccctctcacccactgctgtggccctggccttcgacccaccagccaggaagacaaagctacccagactctcagcccagcctcccccagccaaggtgtcatgctgccttgtggggtgactgaggaaccccagcgactcttttatggcaatgctggctatcggcttcctctccctgccagtttcccagcagtcttgcccattggggagcagccccccgaagggcagtggcaacatcaagcagaggtacagattgcccgaaagcttcagtgcattgcagaccagttccaccggcttcatgtgcagcaacaccagcagaaccaaaatcgtgtgtggtggcagatcctcctcttcctgcacaaccttgctttgaatggagaagagaacaggaacggggcaggccctaggtga

SEQ ID NO: 32 - DNA, 1353 bp, Homo sapiens
  source 1..1353: mol_type = genomic DNA
atggaagggtctaagacgtccaacaacagcaccatgcaggtgagcttcgtgtgccagcgctgcagccagcccctgaaactggacacgagtttcaagatcctggaccgtgtcaccatccaggaactcacagctccattacttaccacagcccaggcgaaaccaggagagacccaggaggaagagactaactcaggagaggagccatttattgaaactcctcgccaggatggtgtctctcgcagattcatccccccagccaggatgatgtccacagaaagtgccaacagcttcactctgattggggaggcatctgatggcggcaccatggagaacctcagccgaagactgaaggtcactggggacctttttgacatcatgtcgggccagacagatgtggatcacccactctgtgaggaatgcacagatactcttttagaccagctggacactcagctcaacgtcactgaaaatgagtgtcagaactacaaacgctgtttggagatcttagagcaaatgaatgaggatgacagtgaacagttacagatggagctaaaggagctggcactagaggaggagaggctgatccaggagctggaagacgtggaaaagaaccgcaagatagtggcagaaaatctcgagaaggtccaggctgaggctgagagactggatcaggaggaagctcagtatcagagagaatacagtgaatttaaacgacagcagctggagctggatgatgagctgaagagtgttgaaaaccagatgcgttatgcccagacgcagctggataagctgaagaaaaccaacgtctttaatgcaaccttccacatctggcacagtggacagtttggcacaatcaataacttcaggctgggtcgcctgcccagtgttcccgtggaatggaatgagattaatgctgcttggggccagactgtgttgctgctccatgctctggccaataagatgggtctgaaatttcagagataccgacttgttccttacggaaaccattcatatctggagtctctgacagacaaatctaaggagctgccgttatactgttctggggggttgcggtttttctgggacaacaagtttgaccatgcaatggtggctttcctggactgtgtgcagcagttcaaagaagaggttgagaaaggcgagacacgtttttgtcttccctacaggatggatgtggagaaaggcaagattgaagacacaggaggcagtggcggctcctattccatcaaaacccagtttaactctgaggagcagtggacaaaagctctcaagttcatgctgacgaatcttaagtggggtcttgcttgggtgtcctcacaattttataacaaatga

SEQ ID NO: 33 - DNA, 945 bp, Homo sapiens
  source 1..945: mol_type = genomic DNA
atggaaggtgtggaacttaaagaagaatggcaagatgaagattttccgatacctttaccagaagatgatagtattgaagcagatatactagctataactggaccagaggaccagcctggctcactagaagttaatggaaataaagtgagaaagaaactaatggctccagacattagcctgacactggatcctagtgatggctctgtattgtcagatgatttggatgaaagtggggagattgacttagatggcttagacacaccgtcagagaatagtaatgagtttgagtgggaagatgatcttccaaaacccaagactactgaagtaattaggaaaggctcaattactgaatacacagcagcagaggaaaaagaagatggacgacgctggcgtatgttcaggattggagaacaggaccacagggttgatatgaaggcaattgaaccctataaaaaagttatcagccatgggggatattatggggatggattaaatgccattgttgtatttgctgtctgtttcatgcctgaaagtagtcagcctaactatagatacctgatggacaatctttttaaatatgttattggcactttggagctattagtagcagaaaactacatgatagtttatttaaatggtgcaacaactcgaagaaaaatgcccagtctgggatggctcaggaaatgttatcagcaaattgatagaaggttacggaaaaatctaaaatccctaatcattgtacatccttcttggtttatcagaacacttctggctgttacaagaccatttattagctcgaaattcagccaaaaaattagatacgtgtttaatttggcagaactagcagaacttgtccccatggaatacgttggcataccagaatgcataaaacaagttgatcaagaacttaatggaaaacaagatgaaccgaaaaatgaacagtaa

SEQ ID NO: 34 - DNA, 660 bp, Homo sapiens
  source 1..660: mol_type = genomic DNA
atgtcgtcccacctagtcgagccgccgccgcccctgcacaacaacaacaacaactgcgaggaaaatgagcagtctctgcccccgccggccggcctcaacagttcctgggtggagctacccatgaacagcagcaatggcaatgataatggcaatgggaaaaatggggggctggaacacgtaccatcctcatcctccatccacaatggagacatggagaagattcttttggatgcacaacatgaatcaggacagagtagttccagaggcagttctcactgtgacagcccttcgccacaagaagatgggcagatcatgtttgatgtggaaatgcacaccagcagggaccatagctctcagtcagaagaagaagttgtagaaggagagaaggaagtcgaggctttgaagaaaagtgcggactgggtatcagactggtccagtagacccgaaaacattccacccaaggagttccacttcagacaccctaaacgttctgtgtctttaagcatgaggaaaagtggagccatgaagaaagggggtattttctccgcagaatttctgaaggtgttcattccatctctcttcctttctcatgttttggctttggggctaggcatctatattggaaagcgactgagcacaccctctgccagcacctactga

SEQ ID NO: 35 - DNA, 57 bp, synthetic construct
  misc_feature 1..57: note = modified human wildtype sequence
  source 1..57: mol_type = other DNA
atgcctggcaagaaggcccggaagaacgcccaaccttctccagctagagcccctgcc

SEQ ID NO: 36 - DNA, 51 bp, synthetic construct
  misc_feature 1..51: note = modified human wildtype sequence
  source 1..51: mol_type = other DNA
atgcctggcaagaaggcccggaagaacgcccaaccttctccagccagagcc

SEQ ID NO: 37 - DNA, 45 bp, synthetic construct
  misc_feature 1..45: note = modified human wildtype sequence
  source 1..45: mol_type = other DNA
cagaagctgctgaacctgatcagcaagctgttctgcagcggcacc

SEQ ID NO: 38 - DNA, 33 bp, synthetic construct
  misc_feature 1..33: note = modified human wildtype sequence
  source 1..33: mol_type = other DNA
aacctgatcagcaagctgttctgcagcggcacc

SEQ ID NO: 39 - DNA, 162 bp, synthetic construct
  misc_feature 1..162: note = modified human wildtype sequence
  source 1..162: mol_type = other DNA
atgcctggcaagaaggcccggaagaacgcccaaccttctccagctagagcccctgccgaactggaagtggaatgtgccacacagctgcggagattcggcgacaagctgaacttccggcagaagctgctgaacctgatcagcaagctgttctgcagcggcacc

SEQ ID NO: 40 - DNA, 162 bp, synthetic construct
  misc_feature 1..162: note = modified human wildtype sequence
  source 1..162: mol_type = other DNA
atgcctggcaagaaggcccggaagaacgcccaaccttctccagctagagcccctgcctcgtccgccgcgcagctcaccgccgcccggctcaaggcgctaggcgacgagctgcaccagcagaagctgctgaacctgatcagcaagctgttctgcagcggcacc

SEQ ID NO: 41 - DNA, 162 bp, synthetic construct
  misc_feature 1..162: note = modified human wildtype sequence
  source 1..162: mol_type = other DNA
atgcctggcaagaaggcccggaagaacgcccaaccttctccagctagagcccctgccctctgggcagcacagcgctatggccgcgagctccggaggatgagtgacgagtttgtggaccagaagctgctgaacctgatcagcaagctgttctgcagcggcacc

SEQ ID NO: 42 - DNA, 162 bp, synthetic construct
  misc_feature 1..162: note = modified human wildtype sequence
  source 1..162: mol_type = other DNA
atgcctggcaagaaggcccggaagaacgcccaaccttctccagctagagcccctgccgacatcatccggaatattgccaggcacctcgcccaggtcggggacagcatggaccgtagccagaagctgctgaacctgatcagcaagctgttctgcagcggcacc

SEQ ID NO: 43 - DNA, 162 bp, synthetic construct
  misc_feature 1..162: note = modified human wildtype sequence
  source 1..162: mol_type = other DNA
atgcctggcaagaaggcccggaagaacgcccaaccttctccagccagagccgacatgaggcccgagatctggattgcccaagagctgagaagaatcggcgacgagttcaacgcctactacgccagacggaacctgatcagcaagctgttctgcagcggcacc

SEQ ID NO: 44 - DNA, 162 bp, synthetic construct
  misc_feature 1..162: note = modified human wildtype sequence
  source 1..162: mol_type = other DNA
atgcctggcaagaaggcccggaagaacgcccaaccttctccagctagagcccctgccttcaaagcctggggcaagttcggaggcgccttttgccggtatgcggatgagatgaaagaacagaagctgctgaacctgatcagcaagctgttctgcagcggcacc

SEQ ID NO: 45 - protein, 54 aa, Unidentified
  VAR_SEQ 1..54
MPGKKARKNAQPSPARAPAELEVECATQLRRFGDKLNFRQKLLNLISKLFCSGT

SEQ ID NO: 46 - protein, 55 aa, Unidentified
  VARIANT 1..55
MPGKKARKNAQPSPARAPAMSSAAQLTAARLKALGDELHQQKLLNLISKLFCSGT

SEQ ID NO: 47 - protein, 54 aa, Unidentified
  VARIANT 1..54
MPGKKARKNAQPSPARAPALWAAQRYGRELRRMSDEFVDQKLLNLISKLFCSGT

SEQ ID NO: 48 - protein, 54 aa, Unidentified
  VARIANT 1..54
MPGKKARKNAQPSPARAPADIIRNIARHLAQVGDSMDRSQKLLNLISKLFCSGT

SEQ ID NO: 49 - protein, 54 aa, Unidentified
  VARIANT 1..54
MPGKKARKNAQPSPARADMRPEIWIAQELRRIGDEFNAYYARRNLISKLFCSGT

SEQ ID NO: 50 - protein, 54 aa, Unidentified
  VARIANT 1..54
MPGKKARKNAQPSPARAPAFKAWGKFGGAFCRYADEMKEQKLLNLISKLFCSGT

SEQ ID NO: 51 - protein, 247 aa, Homo sapiens
MQPILLLLAFLLLPRADAGEIIGGHEAKPHSRPYMAYLMIWDQKSLKRCGGFLIRDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRY

SEQ ID NO: 52 - protein, 54 aa, Unidentified
  VARIANT 1..54
MPGKKARKNAQPSPARAPAELEVECATQERREGDKENFRQKLLNLISKLFCSGT

SEQ ID NO: 53 - protein, 10 aa, Unidentified
  VARIANT 1..10
QERREGDKEN

SEQ ID NO: 54 - DNA, 26 bp, synthetic construct
  variation 1..26
  source 1..26: mol_type = other DNA
agagcggagagagggcgacaaagaga

SEQ ID NO: 55 - DNA, 162 bp, synthetic construct
  variation 1..162
  source 1..162: mol_type = other DNA
atgcctggcaagaaggcccggaagaacgcccaaccttctccagctagagcccctgccgaactggaagtggaatgcgccacacaagagcggagagagggcgacaaagagaacttccggcagaagctgctgaacctgatcagcaagctgttctgcagcggcacc

SEQ ID NO: 56 - DNA, 516 bp, Synthetic construct
  source 1..516: mol_type = other DNA
atggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagacc

(19) United States
(12) Patent Application Publication
(10) Pub. No.: US 2025/0009796 A1
(43) Pub. Date: Jan. 9, 2025

(54) PRO-APOPTOTIC CONSTRUCT AND USE THEREOF

(71) Applicant: UMC UTRECHT HOLDING B.V., Utrecht (NL)

(72) Inventors: Victor Peperzak, Utrecht (NL); Zsolt Sebestyen, Utrecht (NL); Thomas Kimman, Utrecht (NL)

(21) Appl. No.: 18/688,542

(22) PCT Filed: Aug. 30, 2022
(86) PCT No.: PCT/EP2022/074109

§ 371 Date: Mar. 1, 2024

(30) Foreign Application Priority Data
Sep. 2, 2021 (EP) 21194538.1

(51) Int. Cl. A61K 35/17; A61K 39/00; A61P 35/00; C07K 14/47

(52) U.S. Cl. CPC A61K 35/17; A61K 39/4611; A61K 39/4631; A61K 39/46445; A61P 35/00; C07K 14/4747; C07K 2319/055

(57) ABSTRACT

The current disclosure relates to pro-apoptotic molecules with a B-cell lymphoma 2 (BCL-2) homology 3 (BH3) effector domain. The current disclosure furthermore relates to pro-apoptotic constructs wherein the pro-apoptotic molecules are linked to a granule-localizing domain. The pro-apoptotic construct may be transferred from an effector cell to a target cell to induce apoptosis. The current disclosure also relates to the nucleic acid molecules encoding the pro-apoptotic proteins and the uses thereof in a medical therapy such as cancer therapy, including chimeric antigen receptor cell therapy and the like.

### CROSS-REFERENCE TO RELATED APPLICATIONS

This application is a national phase entry under 35 U.S.C. § 371 of International Patent Application PCT/EP2022/074109, filed Aug. 30, 2022, designating the United States of America and published as International Patent Publication WO 2023/031215 A2 on Mar. 9, 2023, which claims the benefit under Article 8 of the Patent Cooperation Treaty of European Patent Application Serial No. 21194538.1, filed Sep. 2, 2021.

### TECHNICAL FIELD

The current disclosure relates to a pro-apoptotic construct that may be used to target cancer cells in a medical therapy, for example, in immune cell transfer therapy.

### STATEMENT REGARDING ELECTRONIC FILING OF A SEQUENCE LISTING

Pursuant to 37 C.F.R. § 1. § 1.834, a Sequence Listing XML file entitled “6VP3091-P35202PC00-ST26 sequence lis.xml,” 68.9 kilobytes in size, generated Feb. 29, 2024, has been submitted via EFS-Web is provided in lieu of a paper copy. This Sequence Listing is hereby incorporated by reference into the specification for its disclosures.

### BACKGROUND

Immunological strategies for cancer treatment comprise the adoptive transfer of cells with engineered anti-tumor specificity. These engineered cells may include chimeric antigen receptor (CAR) T or NK cells, αβ T cell receptor (TCR)-modified T cells and T cells engineered to express a defined γδ TCR (TEGs). Cytotoxic lymphocytes (CLs), such as cytotoxic T-lymphocytes and natural killer cells (NKs), are thought to be particularly efficient in the killing of tumor cells and are typically considered main effector cells in cancer immunotherapy. In addition to their role in cancer treatment, engineered CLs may possibly also be harnessed for treatment of non-malignant diseases including infection, autoimmunity, and in allotransplantation.

CLs use granzymes, in particular, Granzyme B (GzmB), to kill target cells. Granzymes are serine proteases contained in lytic granules where they are co-packed with pore-forming cytolytic proteins such as perforin. Upon CL activation by a target cell, granzyme and perforin are exocytosed from the lytic granule into the synapse between the CL and the target cell. At the target cell membrane, perforin aggregates to form multimeric, transmembrane pores. This allows the entry of granzyme into cytosol of the target cell, where it preferentially induces cell apoptosis by promoting mitochondrial outer membrane permeabilization and/or activating caspases, which cleaves many substrates, including caspase-activated DNase to execute cell death.

Adoptive cellular therapies are in principle an effective form of immune therapy in several types of cancer. However, there are still major problems. First, treating solid tumors with engineered CLs has proven to be inadequate when compared to their efficacy in the treatment of hematologic malignancies. Second, relapse after initial responses remains a major problem (Sterner et al., Blood Cancer J. 2021 Apr. 6; 11(4):69). Although the reason for the suboptimal clinical efficacy remains inconclusive to date, it appears that that tumors have natural mechanisms to resist the killing machinery of engineered CLs.

A number of strategies have been proposed to bypass apoptosis resistance and improve target cell killing by engineered CLs. Modification of engineered CL specificity, enhancing CL activity, modulating interaction of CLs with the microenvironment, including safety measures and promoting the survival, proliferation and trafficking of gene CLs have been considered (Newick et al., Annu. Rev. Med. 2017 Jan. 14; 68:139-152).

Despite these efforts, the proposed methods do not appear to efficiently counteract or bypass the natural mechanisms by which tumor cells resist the killing machinery of CLs.

A current challenge is to overcome tumor resistance to immune cell-mediated killing of cancer cells. A major problem to be solved herein is to enhance the normal cytotoxic capacity of immune cells and the natural pro-apoptotic effectors therein.

### BRIEF SUMMARY

It was found that the resistance of tumor cells to engineered CLs mediated killing is likely related to expression of Serpin B9 by target cells, which render the target cells resistant to the killing activity of granzymes, despite effective targeting. Serpin B9 is the only human protein currently known to be able to inhibit the activity of GzmB and was found to be expressed not only in hematological cancers (e.g., lymphoma, multiple myeloma), but also in solid tumors including liver cancer, breast cancer, lung cancer, and colorectal cancer. The production of natural inhibitors such as serpin B9 to pro-apoptotic proteins could explain the relapse after initial responses using engineered CLs. Moreover, Serpin B9 expression could also explain why, so far, gene engineered T cells were successful in the treatment of hematologic malignancies, but much less so in solid tumors.

It was found that the resistance of cancers to engineered CLs can be bypassed by inactivating pro-survival B-cell lymphoma 2 (BCL-2) family proteins using BH3-only proteins. To this end, modified variants of BCL-2 homology 3 (BH3)-only proteins, i.e., having improved pro-apoptotic potency, were developed. In particular, the use of modified variants of NOXA has been envisaged. NOXA plays an important role in the control of cancers cells as it is induced following genotoxic stress through activation of p53. In addition, due to is small size, NOXA can be readily translocated from a CL effector cell to a tumor target cell. Therefore, there is a desire in the art to further boost the pro-apoptotic activity of NOXA. Normally, NOXA promotes cell killing by neutralizing pro-survival BCL-2 family protein MCL-1. However, NOXA does not inhibit other BCL-2 family proteins (e.g., BCL-2, BCL-B, BCL-W, and BCL-XL), even though many tumor types also depend on expression of these other BCL-2 family proteins for survival. By replacing the BH3 effector domain of NOXA by a BH3 effector domain of another pro-apoptotic protein (preferably the BH3 domain of pro-apoptotic BIM), multiple or even all pro-survival BCL-2 family proteins can be inhibited. By introducing the BH3 effector domain of BIM into NOXA, maximal induction of apoptosis can be achieved. It has been anticipated that all tumor types will be sensitive to this modified version of NOXA having the BIM BH3 effector domain. It has been confirmed that the NOXA(BIM) constructs lead to killing of different types of tumors, e.g., irrespective of MCL-1 sensitivity of the tumor.

It has been established that it is an option to replace the BH3 domain in BH3-only proteins other than NOXA. The modified BH3-only proteins may retain their pro-apoptotic functions that are unrelated to the BH3 effector domain, while also being rendered with a second specific pro-apoptotic function as provided by the BH3 effector domain that inhibits one or more pro-survival BCL-2 family proteins. This may lead to an enhanced pro-apoptotic effect, wherein the optimal strategy may be dependent on the type of cancer and the patient.

Furthermore, it has been shown that tumor eradication by engineered CLs may be largest if the (modified) BH3-only proteins are preceded by a sequence of a granule-localizing domain such as derived from a granzyme protein and the like. The granule-localizing domain naturally localizes to the cytotoxic granules in the CLs. Subsequently, the pro-apoptotic cargo is delivered into the tumor cell following the natural granzyme-perforin pathway. After tumor cell recognition, the granule-localizing protein, together with the pro-apoptotic protein, will be secreted into the synapse between the CL and the tumor cell. Due to the directed secretion into the close environment of the synapse, toxicity to neighboring cells will be minimal. The pro-apoptotic protein will efficiently enter tumor cells through perforin pores and inhibit pro-survival BCL-2 family proteins that sustain tumor cell survival, resulting in apoptosis of the tumor cell. It appears that the use of a granzyme-localizing domain for the delivery of (modified) NOXA is far more effective and causes no or less side effects (e.g., toxicity such as to neighboring cells, off-target pro-apoptotic effects, and/or non-specific killing) as compared to other delivery systems such as the use of a transmembrane-localizing sequence. It has been successfully shown that exogenous delivery of modified pro-apoptotic proteins via transduced CLs induces apoptosis in tumor cells via this mechanism. Incidentally, when comparing tumor cell killing by BH3-only family proteins (e.g., NOXA), granzymes (e.g., granzyme B), or a combination of both, it has been observed that their combined treatment showed increased tumor cell killing capacity compared to the single treatments. This suggests that BH3-only proteins including NOXA and granzymes, including granzyme B, act through different apoptotic pathways that act in concert.

This currently proposed strategy surprisingly overcomes the action of the granzyme inhibitors including Serpin B9 and leads to effective killing of tumor cells. The particularly large potential of the current strategy in cancer treatment is related to the following aspects of the disclosure. First, target cell recognition by engineered CLs brings the required specificity. Second, the activity of pro-apoptotic mediators is confined to the closed environment of the synapse and the intracellular compartment of the target cell, hence toxicity to neighboring cells is circumvented. Third, the pro-apoptotic BH3-only proteins, preferably a modified variant of the pro-apoptotic protein NOXA, induce apoptosis through several routes, at least in part through inhibiting pro-survival BCL-2 family proteins.

### DETAILED DESCRIPTION

It has been shown that a granule-localizing domain, preferably a granzyme, more preferably granzyme B, may act as a chaperone for the cell-specific transfer of a pro-apoptotic protein from an effector cell into a target cell. Furthermore, it has been shown that a (modified variant of a) BH3-only protein transferred into a target cell using such chaperone, readily induces apoptosis in the cancer cell, irrespective of the presence of granzyme (B) inhibitors including Serpin B9. It has been proven that this novel strategy can especially be applied to empower engineered cytotoxic lymphocytes (e.g., engineered T cells or NK cells) to potently kill cancer cells.

In one aspect, the current disclosure relates to a nucleic acid molecule comprising:
1) a first nucleotide sequence encoding a granule-localizing domain;
2) a second nucleotide sequence encoding a pro-apoptotic protein comprising a B-cell lymphoma 2 (BCL-2) homology 3 (BH3) effector domain.

In an embodiment, the nucleic acid molecule as taught herein is a synthetic or recombinant nucleic acid molecule.

The term “nucleic acid molecule” as used herein can be used interchangeably with the term “nucleic acid construct” and wherein the construct can be encoded by one or more (types of) molecules (e.g., RNA, DNA).

In an embodiment, the nucleic acid molecule as taught herein has a length of 10-1,000,000 nucleotides, or 10-100,000 nucleotides, or 10-10,000 nucleotides, or 10-1,000 nucleotides.

The nucleic acid molecule as taught herein preferably encodes for amino acid sequence(s), peptide(s), and/or protein(s). The terms “amino acid sequence” or “protein” or “peptide” refer to molecules consisting of a chain of amino acids, without reference to a specific mode of action, size, three-dimensional structure or origin. A “fragment” or “portion” of thereof may thus still be referred to as an “amino acid sequence” or “protein” or “peptide.”

The term “apoptosis” as used herein relates to a form of programmed cell death that occurs in multicellular organisms. Apoptosis may, for example, be mediated by one of the two best-understood activation mechanisms, i.e., the intrinsic pathway (also called the mitochondrial pathway) and the extrinsic pathway. The intrinsic pathway is typically activated by intracellular signals generated when cells are stressed and depends on the release of proteins from the intermembrane space of mitochondria. The extrinsic pathway is typically activated by extracellular ligands binding to cell-surface death receptors, which leads to the formation of the death-inducing signaling complex. The term “pro-apoptotic” as used herein relates to promoting (or enhancing, increasing) or inducing apoptosis (preferably initiating or giving rise to apoptosis when previously not present). The pro-apoptotic effect (of a substance or therapy) is typically determined by comparing the amount of apoptosis in target cells (e.g., as % apoptosis-related cell death and/or quantification of apoptosis-related markers known in the art) relative to an equivalent untreated control. Any effect size may indicate a pro-apoptotic effect, e.g., an effect size of 1%, 2%, 3%, 4%, 5%, 6%, 7% 8%, 9%, 10%, 20%, 30%, 40%, 50%, 60%, 80%, 90%, 95%, or 100%.

In a preferred embodiment, the BH3-domain encoded by the nucleic acid molecule as taught herein binds a multidomain BCL-2 family protein and/or a BH3 domain-binding groove of a multidomain BCL-2 family protein, such as a (multidomain) pro-apoptotic BCL-2 family protein and/or a BH3 domain-binding groove of a (multidomain) pro-apoptotic BCL-2 family protein, and/or a (multidomain) pro-survival BCL-2 family protein and/or a BH3 domain-binding groove of a (multidomain) pro-survival BCL-2 family protein, preferably a (multidomain) pro-survival BCL-2 family protein and/or a BH3 domain-binding groove of a (multidomain) pro-survival BCL-2 family protein.

The “BCL-2 family of proteins” relates to a family of proteins that are, among others, key regulators of apoptosis. The BCL-2 family of proteins comprises both pro-survival (inhibiting apoptosis) and pro-apoptotic proteins. The BCL-2 family of proteins typically regulate apoptosis by governing mitochondrial outer membrane permeabilization (MOMP) and the pathways involved therein. Mitochondrial outer membrane permeabilization is considered a key step in the intrinsic pathway of apoptosis. The BCL-2 family of proteins is characterized by evolutionarily conserved BCL-2 homology (BH) domains. Generally, all members of the BCL-2 family share a close homology in up to four characteristic regions termed the BH (BCL-2 homology) domains (BH1-BH4). Of these different BH1-BH4 domains, the “BH3 domain” (or BH3 motif) as taught herein refers to the domain that determines the binding specificity for BCL-2 family proteins. As such, the BH3 domain largely determines the interactions between members of the BCL-2 family of proteins in the regulation The BH3 domain is thought to be involved in interactions between the BCL-2 proteins and may be important for the pro-survival and pro-apoptotic functions of the BCL-2 family members and MOMP.

The term “BCL-2 family protein” as used herein relates to a protein belonging to the BCL-2 family of proteins. The term “multidomain BCL-2 family protein” as used herein relates to a BCL-2 family protein having two or more different types of BH domain selected from the BH1-BH4 domains. The term “BH3-only family protein” or “BH3-only protein” as used herein relate to a BCL-2 family protein having only a BH3 domain, thus lacking a BH1, BH2 and BH4 domain.

The BCL-2 family proteins are typically classified into three major subgroups:
1) Pro-survival BCL-2 family proteins, which include, but are not limited to, BCL-2, B-cell lymphoma extra-large (BCL-XL), myeloid cell leukemia 1 (MCL-1), BCL-2 related gene in fetal liver (BFL-1, or A1), BCL-W, and BCL-2-like protein 10 (BCL-2L10, or BCL-B). The pro-survival BCL-2 family proteins typically possess four BH domains (BH 1-BH4), hence are generally considered “multidomain” BCL-2 family proteins. They typically preserve outer mitochondrial membrane integrity by inhibiting the pro-apoptotic members of the BCL-2 family of proteins. In the pro-survival BCL-2 family proteins, the BH1, BH2 and BH3 domains generally interact to form a hydrophobic groove on the surface of the protein. This groove accommodates the BH3 domain of its pro-apoptotic partners and is involved in the pro-survival function of these proteins. The protein “BCL-2” as used herein refers to a single member of the (multidomain) pro-survival BCL-2 family proteins, e.g., as the founding member of the BCL-2 family of proteins;
2) Pro-apoptotic BCL-2 family proteins, which include, but are not limited to, BCL-2 homologous antagonist/killer (BAK), BCL-2-associated X (BAX), and BCL-2 related ovarian killer (BOK). The pro-apoptotic BCL-2 family proteins generally contain three BH domains (BH1, BH2 and BH3), hence are generally considered “multidomain” BCL-2 family proteins. The pro-apoptotic BCL-2 family proteins, foremost BAX and BAK, are effectors of the BCL-2-regulated apoptosis pathway by permeabilizing the mitochondria to allow the release of apoptogenic proteins into the cytosol. This is thought to occur through the formation of pores in the outer mitochondrial membrane. Hence, the pro-apoptotic BCL-2 family proteins have also been referred to as “pore-former” pro-apoptotic proteins.
3) Pro-apoptotic BH3-only proteins, including but not limited to BH3-interacting domain death agonist (BID, also called Bod), truncated BID (tBID), BIM, p53 upregulated modulator of apoptosis (PUMA, also called BBC3), BCL-2/adenovirus E1B 19 kDa protein-interacting protein (BNIP)2, BNIP3, BNIP3L, harakiri (HRK, also called Dp5), BCL-2 interacting killer (BIK, also called Nbk), BCL-2-modifying factor (BMF), Beclin-1, BCL-2 associated agonist of cell death (BAD), and phorbol-12-myristate-13-acetate-induced protein 1 (NOXA). The BH3-only proteins are named for their single conserved BH3 domain. The BH3-only proteins may bind (strongly) to at least one pro-survival BCL-2 family protein. The BH3 domain of BH3-only proteins generally inserts in the hydrophobic groove formed by the BH1, BH2 and BH3 domains at the surface of the pro-survival BCL-2 proteins. The BH3 domain seems to be largely responsible for the binding and inactivation of pro-survival BCL-2 family proteins and is, therefore, considered important for the pro-apoptotic activities of the BH3-only proteins.

The term “BH3 effector domain” as used herein relates to any amino acid sequence (e.g., a domain or motif) that can bind to a groove on multi-domain BCL-2 family protein, preferably a groove on a pro-survival BCL-2 protein, most preferably a groove on one or more of BCL-XL, BCL-W, BCL-2, MCL-1 and BFL-1. Preferably, the binding to a groove is established for the amino acid sequence comprised in a full-length BCL-2 family protein, preferably a full-length BH3-only protein. The term “BH3 effector domain,” as used herein, excludes the BH3 domain of the multi-domain BCL-2 proteins, e.g., that are tightly associated with the mitochondrial membrane and/or inserted into the mitochondrial membrane to induce MOMP and that are typically considered as pore-formers (i.e., multi-domain BCL-2 proteins BAX, BAK, BOK). The term “BH3 effector domain” as used herein preferably excludes the BH3 domain of the multi-domain BCL-2 proteins BAX, BAK, and/or BOK. BAX, BAK, and BOK are typically not suitable to regulate apoptosis by inactivating pro-survival BCL-2 family proteins due to their localization and/or binding affinity to pro-survival BCL-2 family proteins. The term “BH3 effector domain” as used herein includes “BH3-like amino acid sequences,” wherein a BH3-like amino acid sequences is an amino acid sequence not found in wildtype BCL-2 family proteins (in humans), but can nevertheless bind to a groove on multi-domain BCL-2 family protein, preferably a groove on a pro-survival BCL-2 protein, most preferably a groove on one or more of BCL-XL, BCL-W, BCL-2, BCL-B, MCL-1 and BFL-1. For example, PURB has been identified as a protein having a BH3-like amino acid sequence. More specifically, the BH3-like amino acid sequence in PURB binds specifically and with high affinity (e.g., with a K_{D} value of 40 nM) to BCL-2, and not to BCL-XL, BCL-W, MCL-1, and BFL-1 (DeBartolo, J. et al. PLoS Comput. Biol. 2014. PMID: 24967846).

The modified variant of a pro-apoptotic (BH3-only) BCL-2 family protein is preferably functional or “active.”

The term “modified variant” as used herein typically refers to a protein, preferably a pro-apoptotic protein, more preferably a BCL-2 family pro-apoptotic protein, most preferably a BH3-only protein, wherein a BH3 effector domain of another BCL-2 family pro-apoptotic protein is comprised. In addition or alternatively, the BH3 effector domain in the non-modified protein (i.e., wildtype) may be replaced by another BH3 effector domain (i.e., a BH3 effector domain not found in the wildtype protein). As a result of the modification, the modified variant typically has an altered binding affinity to one or more BCL-2 family proteins, preferably multi-domain BCL-2 family proteins, more preferably pro-survival BCL-2 family proteins. In addition or alternatively, as a result of the modification, the modified variant typically has a different pro-apoptotic effect.

This preferably renders the modified variant of NOXA to have a different binding affinity to one or more pro-survival BCL-2 proteins as compared to wildtype NOXA. Normally, NOXA promotes cell killing by neutralizing the pro-survival protein MCL-1. However, NOXA does not inhibit other BCL-2 family proteins. By replacing the BH3 effector domain of NOXA, that is, the domain that is used for binding and inhibiting pro-survival proteins, NOXA can be rendered with an inhibitory effect on other BCL-2 family proteins. To illustrate, replacing the BH3 effector domain of NOXA with the BH3 effector domain of BIM, many pro-survival BCL-2 family proteins are inhibited (at least BCL-2, BCL-XL, BCL-W, MCL-1, BFL-1 and BCL-B) and a highly potent pro-apoptotic effect may be achieved.

“Active” as used herein means that the protein has a pro-apoptotic effect and/or binds one or more pro-survival BCL-2 family-proteins. In contrast, an “inactive” form has no pro-apoptotic effect and/or does not bind a pro-survival BCL-2 family-proteins. An inactive form may be the result of amino acid substitutions in the wildtype protein and/or the result of nucleotide substitutions in the sequence encoding the wildtype protein. In a preferred embodiment of the current disclosure, the modified variant of a pro-apoptotic (BH3-only) BCL-2 family protein as taught herein, is active. In addition or alternatively, the modified variant of a pro-apoptotic (BH3-only) BCL-2 family protein as taught herein is not inactive. Preferably, the modified variant of NOXA as taught herein not an inactive variant of NOXA and/or preferably does not have an amino acid sequence with at least 40, 50, 60, 70, 80, 85, 90, 95, 96, 97, 98, 99, 99.5, 99.9%, or 100% sequence identity with SEQ ID NO:52, and/or preferably does not comprise the amino acid sequence as provided in SEQ ID NO:53. In addition or alternatively, the nucleic acid molecule as taught herein preferably does not comprise a nucleotide sequence with at least 40, 50, 60, 70, 80, 85, 90, 95, 96, 97, 98, 99, 99.5, 99.9%, or 100% sequence identity with SEQ ID NO:54, and/or preferably does not comprise the nucleotide sequence as provided in SEQ ID NO:55.

In an embodiment of the current disclosure, the modified variant of NOXA as taught herein excludes a protein having an amino acid sequence with at least 40, 50, 60, 70, 80, 85, 90, 95, 96, 97, 98, 99, 99.5, 99.9%, or 100% sequence identity with SEQ ID NO:52.

The “binding affinity” as used herein relates to the strength of the binding interaction between a molecule (e.g., a BH3-only protein or a modified variant thereof such as having a substituted BH3 domain) to its binding partner molecule (e.g., a multidomain BCL-2 family protein, preferably a pro-survival BCL-2 family protein). The binding affinity of a protein is generally measured and reported by the equilibrium dissociation constant (K_{d}), which is used to evaluate and rank order strengths of bimolecular interactions. The smaller the K_{d}, the greater the binding affinity of the ligand for its target. The skilled person is well-aware of the different ways to measure the K_{d} value, such as by ELISA, gel-shift assays, pull-down assays, equilibrium dialysis, analytical ultracentrifugation, surface plasmon resonance, and spectroscopic assays. As used herein, the term “binding” indicates that the binding affinity between of a molecule to its binding partner molecule is characterized by a K_{d} value of less than 1000 nM, preferably less than 300 nM. These threshold K_{d} values are typically indicative of a strong binding affinity between a BH3-only protein (or a modified variant thereof), with a binding partner molecule (e.g., herein preferably a pro-survival BCL-2 family protein or a BH3 binding groove thereof), as has been indicated in the state-of-the art literature (Kale et al., Cell Death Differ. 2018 January; 25(1):65-80). It is well-known by the skilled person that the dissociation constant is dependent on the size of the protein used, e.g., as reported by Kale et al. (Cell Death Differ. 2018 January; 25(1):65-80). In the current disclosure, the K_{d} values are preferably determined for a full-length protein, e.g., a full-length BH3-only protein, a full-length modified variant BH3-only protein, or a full-length pro-survival BCL-2 family protein.

In an embodiment, the granule-localizing domain as taught herein is a domain that localizes to a granule, e.g., that localizes the pro-apoptotic protein according to the disclosure to a granule, preferably a lytic (or cytotoxic) granule in a cytotoxic lymphocyte (preferably a cytotoxic T lymphocyte or a NK cell). The term “granule-localizing domain” may be interchanged in the current disclosure with the term “immunological synapse-localizing domain.” The immunological synapse as used herein means the interface between an antigen-presenting cell and/or target cell, preferably a cancer cell, and an effector cell, preferably an (cytotoxic) immune cell. Among the possible other functions of the immunological synapse, it is involved in directing the secretion of the content of the (lytic) granule and/or transfer of the content of the (lytic) granule into an effector cell. Subsequently, the pro-apoptotic cargo is delivered into the tumor cell following the natural granzyme-perforin pathway. After tumor cell recognition, the granule-localizing protein, together with the pro-apoptotic protein, will be secreted into the synapse between the CL and the tumor cell. Due to the directed secretion into the close environment of the synapse, toxicity to neighboring cells will be minimal. The granule-localizing domain in the context of the present disclosure may encompass any protein known to be specifically transported to cytotoxic granules. For example, granule-localizing domains that the skilled person would be familiar and that are suitable for the present disclosure, are at least one or more of perforin, granulysin, granzymes (e.g., A, B, H, M), cathepsin (e.g., C, D, L), chondroitin sulfate proteoglycans, mannose 6-phosphate receptor, H+-ATPase, rylsulfatase, β-Hexosamidase, β-Glucuronidase, CD63, Lamp 1, Lamp 2 (Smyth et al., J. Leukoc. Biol. 2001 July; 70(1):18-29), and serglycin (Metkar et al., Immunity. 2002 March; 16(3):417-28).

In an embodiment, the granule-localizing domain is part of the granzyme-perforin pathway. In an embodiment, the granule-localizing domain is not part of the granzyme-perforin pathway.

In a preferred embodiment, the granule-localizing domain is (a leader peptide of) granzyme A, granzyme B, granzyme H, granzyme K, granzyme M, granulysin, serglycin, and/or perforin.

In an embodiment, granzyme B as taught herein as has at least 40, 50, 60, 70, 80, 85, 90, 95, 96, 97, 98, 99, 99.5, 99.9%, or 100% sequence identity with SEQ ID NO:51.

The term “effector cell” refers to a cell that mediates the killing of the target cell as taught herein. The effector cell preferably is an immune cell as taught herein, more preferably a cytotoxic lymphocyte, more preferably a cytotoxic T cell or NK cell, most preferably a chimeric antigen receptor (CAR) T cell or NK cell. In addition or alternatively, the effector cell preferably expresses the molecule encoded by the nucleic acid molecule as taught herein. The term “target cell” as used herein refers to a cell to which the (pro-apoptotic) therapy as taught herein is preferably targeted. In addition or alternatively, a target cell is a cell to which the granule-localizing peptide as disclosed herein and/or the pro-apoptotic protein comprising a BH3 effector domain as disclosed herein is transferred to, e.g., via the granzyme-perforin pathway. In addition or alternatively, the target cell refers to a cell where the pro-apoptotic protein comprising a BH3 effector domain as disclosed herein acts upon, i.e., the cell wherein apoptosis is induced. If the effector cell is a cytotoxic T cell, the target cell is preferably a cell bearing an appropriate antigenic complex (peptide-MHC) recognized by their T cell receptor and activates the cytotoxic T cell to release lytic proteins (e.g., a granzyme, perforin). The target cell as used herein is typically an infected host cell, preferably a virus-infected host cell, or a hyperproliferative cell, preferably a tumor and/or cancer cell.

In a preferred embodiment, the granule-localizing domain as taught herein (e.g., encoded by the first nucleotide sequence comprised in the nucleic acid molecule as taught herein) is:
a granzyme protein or a leader peptide thereof, wherein the granzyme protein may be one or more selected from any known human granzyme (such as granzyme A, granzyme B, granzyme H, granzyme K, and granzyme M, preferably granzyme B);
perforin or a leader peptide thereof;
serglycin or a leader peptide thereof;
granulysin or a leader peptide thereof, and/or
an N-Acetylglucosamine-1 (GlcNAc-1) phosphotransferase binding domain,
   and/or the first nucleotide sequence as taught herein is:
a nucleotide sequence encoding granzyme A, preferably having at least 40, 50, 60, 70, 80, 85, 90, 95, 96, 97, 98, 99, 99.5, 99.9%, or 100% sequence identity with SEQ ID NO:1, or a nucleotide sequence encoding a leader peptide of granzyme A, preferably having at least 40, 50, 60, 70, 80, 85, 90, 95, 96, 97, 98, 99, 99.5, 99.9%, or 100% sequence identity with SEQ ID NO:2;
a nucleotide sequence encoding granzyme B, preferably having at least 40, 50, 60, 70, 80, 85, 90, 95, 96, 97, 98, 99, 99.5, 99.9%, or 100% sequence identity with SEQ ID NO:3, or a nucleotide sequence encoding a leader peptide of granzyme B, preferably having at least 40, 50, 60, 70, 80, 85, 90, 95, 96, 97, 98, 99, 99.5, 99.9%, or 100% sequence identity with SEQ ID NO:4;
a nucleotide sequence encoding granzyme H, preferably having at least 40, 50, 60, 70, 80, 85, 90, 95, 96, 97, 98, 99, 99.5, 99.9%, or 100% sequence identity with SEQ ID NO:5, or a nucleotide sequence encoding a leader peptide of granzyme H, preferably having at least 40, 50, 60, 70, 80, 85, 90, 95, 96, 97, 98, 99, 99.5, 99.9%, or 100% sequence identity with SEQ ID NO:6;
a nucleotide sequence encoding granzyme K, preferably having at least 40, 50, 60, 70, 80, 85, 90, 95, 96, 97, 98, 99, 99.5, 99.9%, or 100% sequence identity with SEQ ID NO:7, or a nucleotide sequence encoding a leader peptide of granzyme K, preferably having at least 40, 50, 60, 70, 80, 85, 90, 95, 96, 97, 98, 99, 99.5, 99.9%, or 100% sequence identity with SEQ ID NO:8;
a nucleotide sequence encoding granzyme M, preferably having at least 40, 50, 60, 70, 80, 85, 90, 95, 96, 97, 98, 99, 99.5, 99.9%, or 100% sequence identity with SEQ ID NO:9, or a nucleotide sequence encoding a leader peptide of granzyme M, preferably having at least 40, 50, 60, 70, 80, 85, 90, 95, 96, 97, 98, 99, 99.5, 99.9%, or 100% sequence identity with SEQ ID NO:10;
a nucleotide sequence encoding granulysin, preferably having at least 40, 50, 60, 70, 80, 85, 90, 95, 96, 97, 98, 99, 99.5, 99.9%, or 100% sequence identity with SEQ ID NO:11, or a nucleotide sequence encoding leader peptide of granulysin, preferably having at least 40, 50, 60, 70, 80, 85, 90, 95, 96, 97, 98, 99, 99.5, 99.9%, or 100% sequence identity with SEQ ID NO:12;
a nucleotide sequence encoding serglycin, preferably having at least 40, 50, 60, 70, 80, 85, 90, 95, 96, 97, 98, 99, 99.5, 99.9%, or 100% sequence identity with SEQ ID NO:13, or a nucleotide sequence encoding a leader peptide of serglycin, preferably having at least 40, 50, 60, 70, 80, 85, 90, 95, 96, 97, 98, 99, 99.5, 99.9%, or 100% sequence identity with SEQ ID NO:14;
a nucleotide sequence encoding perforin, preferably having at least 40, 50, 60, 70, 80, 85, 90, 95, 96, 97, 98, 99, 99.5, 99.9%, or 100% sequence identity with SEQ ID NO:15, or a nucleotide sequence encoding a leader peptide of perforin, preferably having at least 40, 50, 60, 70, 80, 85, 90, 95, 96, 97, 98, 99, 99.5, 99.9%, or 100% sequence identity with SEQ ID NO:16; and/or
a nucleotide sequence encoding for an N-Acetylglucosamine-1 (GlcNAc-1) phosphotransferase binding domain, preferably having at least 40, 50, 60, 70, 80, 85, 90, 95, 96, 97, 98, 99, 99.5, 99.9%, or 100% sequence identity with SEQ ID NO:17.

A “leader peptide” as used herein can lead a protein wherein it is comprised to a cellular organelle, such as a (lytic) granule. A leader peptide as used herein may relate to a peptide (generally 16-30 amino acids in length), typically present at the N-terminus (but occasionally at the C-terminus) of proteins that are destined toward the secretory pathway. The proteins having a leader peptide include those that reside either inside organelles such as the endoplasmic reticulum, Golgi, endosomes, and/or the lytic (granule). In addition or alternatively, the proteins having a leader peptide include those secreted from the cell and/or inserted into cellular membranes. A leader peptide is sometimes also referred to as a signal peptide, a signal sequence, a targeting signal, a localization signal, a localization sequence, a transit peptide, or a leader sequence), and may thus be used interchangeably therewith. Proteins having a leader peptide are generally localized to the endoplasmic reticulum, Golgi, endosomes, and/or the lytic (granule) during and/or after translation. In contrast, proteins not having a leader peptide are generally localized into the cytosol during the entire translation and/or after translation. In the current disclosure, the use of a leader peptide as a granule-localizing domain, preferably a leader peptide of a granzyme, most preferably a leader peptide of granzyme B, may promote the localization of certain cargo (e.g., a pro-apoptotic protein as taught herein) to the (lytic granule), e.g., without using a full-length protein as a granule-localizing domain.

It is considered that the natural configuration of a granzyme, preferably granzyme B, allows the most efficient localization of the protein plus cargo into the (lytic) granules and subsequently into target cells. The granule-localizing domain as taught herein hence preferably is full-length granzyme, preferably a full-length granzyme B.

In an embodiment, the granule-localizing domain is an inactivated form of a full-length granzyme, preferably an inactivated form of a full-length granzyme B, which, for example, allows localization of any of the pro-apoptotic proteins as taught herein into (lytic) granules, but in absence of a functional granzyme.

In an embodiment, the granule-localizing domain is a domain comprising both an endoplasmic reticulum(ER)-Golgi localization domain (e.g., a granzyme signal sequence) and a (lytic) granule localization domain (e.g., a sorting motif including an N-linked glycosylation site) such as present in granzyme (B). This allows to localize to and/or shuttles cargo into the (lytic) granules.

In a preferred embodiment, the BH3 effector domain as taught herein is a BH3 effector domain of a BH3-only pro-apoptotic protein and/or a BH3 effector domain of a purine rich element binding protein (PUR), preferably PURbeta (PURB), wherein the BH3-only pro-apoptotic protein is preferably chosen from BCL-2-like protein 11 (BIM), BH3-interacting domain death agonist (BID), BCL-2 interacting killer (BIK), p53 upregulated modulator of apoptosis (PUMA), BCL-2 associated agonist of cell death (BAD), phorbol-12-myristate-13-acetate-induced protein 1 (NOXA), BCL-2-modifying factor (BMF), harakiri (HRK), Beclin-1, BCL-2/adenovirus E1B 19 kDa protein-interacting protein (BNIP)2, BNIP3, and/or BNIP3L, and/or wherein the BH3 effector domain is encoded by a nucleotide sequence having at least 40, 50, 60, 70, 80, 85, 90, 95, 96, 97, 98, 99, 99.5, 99.9%, or 100% sequence identity with:
SEQ ID NO:18 (e.g., the BH3 effector domain of NOXA);
SEQ ID NO:19 (e.g., the BH3 effector domain of HRK);
SEQ ID NO:20 (e.g., the BH3 effector domain of BAD);
SEQ ID NO:21 (e.g., the BH3 effector domain of BID);
SEQ ID NO:22 (e.g., the BH3 effector domain of BIM); and/or
SEQ ID NO:23 (e.g., the BH3 effector domain of NOXA).

It is considered that PURB and/or the BH3 effector domain of PURB may have a pro-apoptotic effect by binding the pro-survival BCL-2 family protein BCL-2. PURB normally does not interact with other BCL-2 family members (e.g., that are mostly localized in the cytoplasm and mitochondria) and/or directly play a role in the intrinsic pathway of apoptosis.

The BH3 effector domain of PURB was found as the only candidate with specific and high affinity binding to BCL-2 only (DeBartolo, J. et al., PLoS Comput. Biol. 2014. PMID: 24967846).

Modified variants of pro-apoptotic (BH3-only) BCL-2 family protein have been developed and produced, i.e., wherein the wildtype BH3 effector domain is replaced by a BH3 effector domain of another pro-apoptotic (BH3-only) BCL-2 family protein. The BH3 effector domain replacement leads to an altered binding affinity of the protein for the different types of pro-survival BCL-2 family proteins and their inactivation, hereby regulating pro-apoptotic activity. In particular, replacing the BH3 effector domain of a pro-apoptotic (BH3-only) BCL-2 family protein with a BH3 effector domain derived from BAD, BID, or BIM, may lead to the inactivation of multiple pro-survival BCL-2 family proteins and high pro-apoptotic potency. Pro-apoptotic (BH3-only) BCL-2 family protein having a BH3 effector domain of BIM is considered to have the largest therapeutic effect in cancer eradication, since they may inactivate all pro-survival BCL-2 family proteins (i.e., BCL-2, BCL-XL, BCL-W, MCL-1, BFL-1 and BCL-B).

The modified variants of pro-apoptotic (BH3-only) BCL-2 family proteins as disclosed herein provide the skilled person (e.g., the medical practitioner) with an arsenal of tools to apply for more effective treatment of different types of cancer. The pro-apoptotic variants may retain their pro-apoptotic functions that are unrelated to the BH3 effector domain, while also being rendered with a second specific pro-apoptotic function as provided by the BH3 effector domain that inhibits one or more pro-survival BCL-2 family proteins.

This may lead to an enhanced pro-apoptotic effect, wherein the optimal strategy may be dependent on the type of cancer and the patient. To illustrate, in one type of cancer or patient, the therapy may be maximized by selective inhibition of only one specific pro-survival BCL-2 family protein. In another type of cancer or patient, the therapy may be maximized by inhibition of multiple/all pro-survival BCL-2 family proteins. Moreover, the optimal (BH3-only) pro-apoptotic protein serving as a backbone for the modified variant may be dependent on the type of cancer or patient.

It is considered that a BH3-only protein having a BH3 effector domain of NOXA binds MCL-1 and/or BFL-1, and/or does not bind BCL-2, BCL-XL, BCL-Wand BCL-B. In addition or alternatively, it is considered that this BH3-only protein has a moderate pro-apoptotic activity.

It is considered that a BH3-only protein having a BH3 effector domain of PURB binds BCL-2 and/or does not bind BCL-XL, BCL-W, MCL-1, BFL-1 and BCL-B. In addition or alternatively, it is considered that this BH3-only protein has a moderate/high pro-apoptotic activity.

It is considered that a BH3-only protein having a BH3 effector domain of HRK binds BCL-XL and/or does not bind BCL-2, BCL-W, MCL-1, BFL-1 and BCL-B. In addition or alternatively, it is considered that this BH3-only protein has a moderate/high pro-apoptotic activity.

It is considered that a BH3-only protein having a BH3 effector domain of BAD binds BCL-2, BCL-XL, and BCL-W, and/or does not bind MCL-1, BFL-1 and BCL-B. In addition or alternatively, it is considered that this BH3-only protein has a high pro-apoptotic activity.

It is considered that a BH3-only protein having a BH3 effector domain of BID binds BCL-2, BCL-XL, BCL-W, MCL-1, and BFL-1 and/or does not bind BCL-B. In addition or alternatively, it is considered that this BH3-only protein has a high/very high pro-apoptotic activity.

It is considered that a BH3-only protein having a BH3 effector domain of BIM binds BCL-2, BCL-XL, BCL-W, MCL-1, BFL-1, and BCL-B. In addition or alternatively, it is considered that this BH3-only protein has a very high pro-apoptotic activity.

In a preferred embodiment, the second nucleotide sequence as taught herein encodes for a BH3-only pro-apoptotic protein, wherein the BH3 effector domain is substituted by a BH3 effector domain of a second protein, preferably a second BH3-only pro-apoptotic protein, wherein the second protein is preferably chosen from:
NOXA, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:24;
HRK, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:25;
BAD, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:26;
BID, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:27;
BIM, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:28;
BIK, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:29;
PUMA, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:30;
BMF, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:31;
Beclin-1, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:32;
BNIP2, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:33;
BNIP3;
BNIP3L, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:34,
wherein preferably the BH3 effector domain is substituted by:
a BH3 effector domain of NOXA, preferably encoded by a nucleotide sequence having at least 40, 50, 60, 70, 80, 85, 90, 95, 96, 97, 98, 99, 99.5, 99.9%, or 100% sequence identity with SEQ ID NO:18 (wherein NOXA is not the BH3-only pro-apoptotic protein);
a BH3 effector domain of HRK, preferably encoded by a nucleotide sequence having at least 40, 50, 60, 70, 80, 85, 90, 95, 96, 97, 98, 99, 99.5, 99.9%, or 100% sequence identity with SEQ ID NO:19 (wherein HRK is not the BH3-only pro-apoptotic protein);
a BH3 effector domain of BAD, preferably encoded by a nucleotide sequence having at least 40, 50, 60, 70, 80, 85, 90, 95, 96, 97, 98, 99, 99.5, 99.9%, or 100% sequence identity with SEQ ID NO:20 (wherein BAD is not the BH3-only pro-apoptotic protein);
a BH3 effector domain of BID, preferably encoded by a nucleotide sequence having at least 40, 50, 60, 70, 80, 85, 90, 95, 96, 97, 98, 99, 99.5, 99.9%, or 100% sequence identity with SEQ ID NO:21 (wherein BID is not the BH3-only pro-apoptotic protein);
a BH3 effector domain of BIM, preferably encoded by a nucleotide sequence having at least 40, 50, 60, 70, 80, 85, 90, 95, 96, 97, 98, 99, 99.5, 99.9%, or 100% sequence identity with SEQ ID NO:22 (wherein BIM is not the BH3-only pro-apoptotic protein);
a BH3 effector domain of PURB, preferably encoded by a nucleotide sequence having at least 40, 50, 60, 70, 80, 85, 90, 95, 96, 97, 98, 99, 99.5, 99.9%, or 100% sequence identity with SEQ ID NO:23 (wherein PURB is not the BH3-only pro-apoptotic protein;
a BH3 effector domain of PUMA (wherein PUMA is not the BH3-only pro-apoptotic protein);
a BH3 effector domain of BMF (wherein BMF is not the BH3-only pro-apoptotic protein);
a BH3 effector domain of Beclin-1 (wherein Beclin-1 is not the BH3-only pro-apoptotic protein);
a BH3 effector domain of BNIP2 (wherein BNIP2 is not the BH3-only pro-apoptotic protein);
a BH3 effector domain of BNIP3 (wherein BNIP3 is not the BH3-only pro-apoptotic protein); or
a BH3 effector domain of BNTP3L (wherein BNIP3L is not the BH3-only pro-apoptotic protein).

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is NOXA, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:24,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is HRK, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:25,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence in the context of the present disclosure encodes a pro-apoptotic protein preferably comprising a BH3 effector domain.

In an embodiment, the second nucleotide sequence in the context of the present disclosure encodes a pro-apoptotic protein wherein the pro-apoptotic protein is NOXA, and/or a pro-apoptotic protein with at least 40%, or 50%, or 60%, or 70%, or 80%, 85%, 90%, or 95%, 99% or 100% sequence identity with SEQ ID NO:45, and/or a pro-apoptotic protein encoded by a nucleotide sequence having at least 40%, or 50%, or 60%, or 70%, or 80%, 85%, 90%, or 95%, 99% or 100% sequence identity with SEQ ID NO:24.

In an embodiment, the second nucleotide sequence in the context of the present disclosure encodes a pro-apoptotic protein, wherein the BH3 effector domain is substituted by:
a BH3 effector domain of harakiri (HRK), preferably encoded by a nucleotide sequence having at least 70%, 80%, 90%, or 95%, 99% or 100% sequence identity with SEQ ID NO:19,
a BH3 effector domain of BCL-2 associated agonist of cell death (BAD), preferably encoded by a nucleotide sequence having at least 70%, 80%, 90%, or 95%, 99% or 100% sequence identity with SEQ ID NO:20,
a BH3 effector domain of BH3-interacting domain death agonist (BID), preferably encoded by a nucleotide sequence having at least 70%, 80%, 90%, or 95%, 99% or 100% sequence identity with SEQ ID NO:21,
a BH3 effector domain of BCL-2-like protein 11 (BIM), preferably encoded by a nucleotide sequence having at least 70%, 80%, 90%, or 95%, 99% or 100% sequence identity with SEQ ID NO:22, or
a BH3 effector domain of PURbeta (PURB), preferably encoded by a nucleotide sequence having at least 70%, 80%, 90%, or 95%, 99% or 100% sequence identity with SEQ ID NO:23.

In the context of the present disclosure, the term “wherein the BH3 effector domain is substituted” or “substitution of the BH3 effector domain” means that the BH3 effector domain that is native to the pro-apoptotic protein, in other words original or endogenous or naturally occurring BH3 effector domain of the respective pro-apoptotic protein, is substituted by a BH3 effector domain that is not native to the pro-apoptotic protein. The BH3 effector domain that is native to the pro-apoptotic protein preferably has the indicated % sequence identity with one of the sequences according to SEQ ID NOS;18-23 and/or having the indicated % sequence identity with the BH3 effector domain that can be derived from SEQ ID NOS;45-50 (e.g., by excluding the constant amino acid sequences common in SEQ ID NOS;45-50).

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is BAD, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:26,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is preferably chosen from BID, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:27,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is preferably chosen from BIK, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:29,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is preferably chosen from PUMA, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:30,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is preferably chosen from BMF, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:31,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is preferably chosen from Beclin-1, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:32,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is preferably chosen from BNIP2, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:33,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is preferably chosen from BNIP3,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

In an embodiment, the second nucleotide sequence as taught herein encodes for a BH3-only pro-apoptotic protein, wherein the BH3 effector domain is substituted by a BH3 effector domain of BIM,
wherein the BH3-only pro-apoptotic protein is preferably chosen from BNIP3L, and/or a BH3-only protein encoded by a nucleotide sequence having at least 40, 50, 60, 70, 80, 85, 90, 95, 96, 97, 98, 99, 99.5, 99.9%, or 100% sequence identity with SEQ ID NO:34,
wherein the BH3 effector domain of BIM is preferably encoded by a nucleotide sequence having at least 40, 50, 60, 70, 80, 85, 90, 95, 96, 97, 98, 99, 99.5, 99.9%, or 100% sequence identity with SEQ ID NO:22.

It is considered that the activation of the intrinsic apoptosis pathway may induce apoptosis in cancer, while overcoming resistance due to natural inhibitors of apoptosis such as Serpin B9. It is considered that the intrinsic pathway of apoptosis is most preferably induced by selecting pro-apoptotic proteins that regulate the activity of specific pro-survival BCL-2 family proteins. It is considered that the most effective way to achieve this is the use of BH3-only pro-apoptotic proteins and/or modified variants thereof, as disclosed herein.

The use of BH3-only proteins and/or modified variants thereof has been identified as a more potent strategy in killing cancer cells as compared to the use of other (classes of) pro-apoptotic proteins. It has been shown that BH3-only proteins and/or modified variants thereof kill cancer cells irrespective of the presence of inhibitors of apoptosis such as Serpin B9, which is unknown for other (classes of) pro-apoptotic proteins. The advantages of BH3-only proteins compared to other types of pro-apoptotic proteins are further illustrated below:
As a first example, caspases are well-known proteases involved in initiating apoptosis. However, delivery of caspases by means of a granule-localizing domain may not lead to apoptosis. Caspases are only active after cleavage by an activated upstream molecule. Thus, introducing full-length caspases alone are not effective unless apoptosis is induced upstream. On the other hand, the delivery of activated/cleaved caspases in the granules may already degrade various other/target molecules in the cytotoxic lymphocytes, which leads, for example, to high toxicity and complications. BH3-only proteins and/or modified variants specifically induce apoptosis in cancer cells and does not induce toxicity.
As a second example, delivery of multidomain BCL-2 family proteins (e.g., so-called pore-formers) such as BAX and BAK by means of a granule-localizing domain may also not lead to apoptosis. BAK and/or BAX insert themselves into membranes (as they normally do in mitochondria). Hence, this would lead to pore formation in the granules in cytotoxic lymphocytes and that leads, for example, to high toxicity and complications. BH3-only proteins and/or modified variants specifically induce apoptosis in cancer cells and does not induce toxicity.
As a third example, delivery of pro-apoptotic molecules involved in the death receptor pathway by means of a granule-localizing domain may also not lead to apoptosis. Essential molecules downstream of death receptor signaling (FADD, TRAIL2) are frequently mutated in cancer cells. In addition, CAR T cells have shorter contacts with target cells as compared to conventional T cells, therefore the granzyme-perforin pathway plays a more important role (Davenport et al. PNAS 2018. PMID: 29440406). It has also been shown that cytotoxic lymphocytes first kill cancer cells via the content of lytic granules and only later (in serial killing) via death receptors (Prager et al. JEM 2019. PMID: 31270246).

The use of a modified variant of NOXA has been considered as a highly effective way of killing cancer cells, even in the presence of apoptosis inhibitors such as Serpin B9. The term “modified variant of NOXA” as used herein refers to a NOXA protein wherein the BH3 effector domain is replaced by a BH3 effector domain not found in wildtype NOXA. In addition or alternatively, the “modified variant of NOXA” may have a non-NOXA BH3-effector, preferably a BH3-effector domain that is not wildtype of NOXA. This preferably renders the modified variant of NOXA to have a different binding affinity to one or more pro-survival BCL-2 proteins as compared to wildtype NOXA. Normally, NOXA promotes cell killing by neutralizing the pro-survival protein MCL-1, and to a lesser extent BFL-1. However, NOXA does not inhibit other BCL-2 family proteins. By replacing the BH3 effector domain of NOXA, that is, the domain that is used for binding and inhibiting pro-survival proteins, NOXA can be rendered with an inhibitory effect on other BCL-2 family proteins. To illustrate, replacing the BH3 effector domain of NOXA with the BH3 effector domain of BIM, many pro-survival BCL-2 family proteins are inhibited (at least BCL-2, BCL-XL, BCL-W, MCL-1, BFL-1 and BCL-B) and highly potent pro-apoptotic effect may be achieved.

NOXA was identified as the most potent BH3-only protein as part of the strategy of using a modified BH3-only protein. NOXA has by far the smallest size of all BCL-2 family proteins (Gross et al. Cell Death Differ. 2017. PMID: 28234359), while it remains highly functional to induce apoptosis like other BH3-only proteins. In addition, NOXA does not require cleavage to become activated (like BID into truncated BID, or tBID) and exists only in a single isoform. The small size of NOXA as a backbone for the modified BH3-only protein has several advantages. First, it was considered that it makes cloning and expression easier and/or better, both when used as a separate vector or when integrated into an existing CAR vector. Second, the transfer through perforin-pores of small unmodified proteins like NOXA is expected to be more efficient than larger proteins, such as other full-length BH3-only proteins. This would especially be the case when still connected to granzyme B, if the cleavage of the two proteins is suboptimal. A combined Granzyme B-NOXA protein is still expected to pass perforin pores if cleavage is suboptimal. Finally, it was considered that NOXA plays an important role in the natural control of cancer cells as it is induced following genotoxic stress through activation of p53. Therefore, there is a desire in the art to further boost the pro-apoptotic activity of NOXA. It is anticipated that all tumor types will be sensitive to this modified version of NOXA having the BIM BH3 effector domain. Combined, NOXA was identified as by far the most effective option to use in the various strategies as part of the present disclosure.

In a preferred embodiment, the BH3-only protein as taught herein, including the modified variant of a BH3-only protein as taught herein, preferably has a length of 5-250 amino acids, preferably a length of 5-150 amino acids, more preferably a length of 5-75 amino acids, most preferably a length of 5-55 amino acids.

In a preferred embodiment, the BH3-only protein as taught herein, including the modified variant of a BH3-only protein as taught herein, does not require cleavage to become activated and/or to have a pro-apoptotic effect.

In a preferred embodiment, the BH3-only protein as taught herein, including the modified variant of a BH3-only protein as taught herein, only exists in a single isoform (preferably in humans).

In a preferred embodiment, the BH3-only protein as taught herein, including the modified variant of a BH3-only protein as taught herein, is PUMA or NOXA.

In one aspect, the current disclosure relates to a nucleic acid molecule encoding a pro-apoptotic protein, wherein the pro-apoptotic protein is encoded by a nucleotide sequence comprising:
1) a BH3 effector domain encoding sequence;
2) an upstream domain encoding sequence that is located upstream of the BH3 effector domain encoding sequence;
3) a downstream domain encoding sequence that is located downstream of the BH3 effector domain encoding sequence,
wherein the upstream domain encoding sequence encodes:
   an upstream domain of NOXA, preferably having at least 40, 50, 60, 70, 80, 85, 90, 95, 96, 97, 98, 99, 99.5, 99.9%, or 100% sequence identity with SEQ ID NO:35 and/or SEQ ID NO:36,
and/or wherein the downstream domain encoding sequence encodes:
   a downstream domain of NOXA, preferably having at least 40, 50, 60, 70, 80, 85, 90, 95, 96, 97, 98, 99, 99.5, 99.9%, or 100% sequence identity with SEQ ID:37 and/or SEQ ID:38;
and/or wherein the BH3 effector domain encoding sequence encodes:
   a BH3 effector domain of HRK, preferably having at least 40, 50, 60, 70, 80, 85, 90, 95, 96, 97, 98, 99, 99.5, 99.9%, or 100% sequence identity with SEQ ID NO:19;
   a BH3 effector domain of BAD, preferably having at least 40, 50, 60, 70, 80, 85, 90, 95, 96, 97, 98, 99, 99.5, 99.9%, or 100% sequence identity with SEQ ID NO:20;
   a BH3 effector domain of BID, preferably having at least 40, 50, 60, 70, 80, 85, 90, 95, 96, 97, 98, 99, 99.5, 99.9%, or 100% sequence identity with SEQ ID NO:21;
   a BH3 effector domain of BIM, preferably having at least 40, 50, 60, 70, 80, 85, 90, 95, 96, 97, 98, 99, 99.5, 99.9%, or 100% sequence identity with SEQ ID NO:22;
   a BH3 effector domain of PURB, preferably having at least 40, 50, 60, 70, 80, 85, 90, 95, 96, 97, 98, 99, 99.5, 99.9%, or 100% sequence identity with SEQ ID NO:23;
   a BH3 effector domain of PUMA;
   a BH3 effector domain of BMF;
   a BH3 effector domain of Beclin-1;
   a BH3 effector domain of BNIP2;
   a BH3 effector domain of BNIP3; and/or
   a BH3 effector domain of BNIP3L.

The downstream domain as disclosed herein preferably comprises and/or functions as:
a mitochondrial targeting domain (MTD) and/or a pronecrotic domain. In addition or alternatively, the downstream domain as disclosed herein preferably (comprises a domain that) functions as an inducer of mitochondrial fragmentation and/or delivery to mitochondria.

Preferably, the downstream domain in the context of the present disclosure comprises a MTD as according to disclosed by Han et al. (Cell Death & Disease volume 10, Article number: 519 (2019), incorporated by reference). The MTD may further promote cell death induced by the BH3 effector domain and/or causing mitochondrial damage.

In a preferred embodiment, the nucleotide sequence comprising a BH3 effector domain encoding sequence, an upstream domain encoding sequence, and a downstream domain encoding sequence, as taught herein, comprises:
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of HRK, preferably having at least 40, 50, 60, 70, 80, 85, 90, 95, 96, 97, 98, 99, 99.5, 99.9%, or 100% sequence identity with SEQ ID NO:19, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37, preferably the nucleotide sequence has at least 40, 50, 60, 70, 80, 85, 90, 95, 96, 97, 98, 99, 99.5, 99.9%, or 100% sequence identity with SEQ ID NO:40;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of BAD, preferably having at least 40, 50, 60, 70, 80, 85, 90, 95, 96, 97, 98, 99, 99.5, 99.9%, or 100% sequence identity with SEQ ID NO:20, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37, preferably the nucleotide sequence has at least 40, 50, 60, 70, 80, 85, 90, 95, 96, 97, 98, 99, 99.5, 99.9%, or 100% sequence identity with SEQ ID NO:41;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of BID, preferably having at least 40, 50, 60, 70, 80, 85, 90, 95, 96, 97, 98, 99, 99.5, 99.9%, or 100% sequence identity with SEQ ID NO:21, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37, preferably the nucleotide sequence has at least 40, 50, 60, 70, 80, 85, 90, 95, 96, 97, 98, 99, 99.5, 99.9%, or 100% sequence identity with SEQ ID NO:42;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:36, a BH3 effector domain encoding sequence of BIM, preferably having at least 40, 50, 60, 70, 80, 85, 90, 95, 96, 97, 98, 99, 99.5, 99.9%, or 100% sequence identity with SEQ ID NO:22, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:38, preferably the nucleotide sequence has at least 40, 50, 60, 70, 80, 85, 90, 95, 96, 97, 98, 99, 99.5, 99.9%, or 100% sequence identity with SEQ ID NO:43;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of PURB, preferably having at least 40, 50, 60, 70, 80, 85, 90, 95, 96, 97, 98, 99, 99.5, 99.9%, or 100% sequence identity with SEQ ID NO:23, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37, preferably the nucleotide sequence has at least 40, 50, 60, 70, 80, 85, 90, 95, 96, 97, 98, 99, 99.5, 99.9%, or 100% sequence identity with SEQ ID NO:44;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of BIK, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of PUMA, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of BMF, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of Beclin-1, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of BNIP2, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of BNIP3, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:35, a BH3 effector domain encoding sequence of BNIP3L, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:37;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:36, a BH3 effector domain encoding sequence of BIK, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:38;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:36, a BH3 effector domain encoding sequence of PUMA, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:38;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:36, a BH3 effector domain encoding sequence of BMF, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:38;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:36, a BH3 effector domain encoding sequence of Beclin-1, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:38;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:36, a BH3 effector domain encoding sequence of BNIP2, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:38;
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:36, a BH3 effector domain encoding sequence of BNIP3, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:38; or
an upstream domain encoding sequence of NOXA, preferably having at least 40, 50, 60, 70, 80, 85, 90, 95, 96, 97, 98, 99, 99.5, 99.9%, or 100% sequence identity with SEQ ID NO:36, a BH3 effector domain encoding sequence of BNIP3L, and a downstream domain encoding sequence of NOXA, preferably having at least 40, 50, 60, 70, 80, 85, 90, 95, 96, 97, 98, 99, 99.5, 99.9%, or 100% sequence identity with SEQ ID NO:38.

In a preferred embodiment, the nucleotide sequence comprising a BH3 effector domain encoding sequence, an upstream domain encoding sequence, and a downstream domain encoding sequence, as taught herein, comprises a further nucleotide sequence that encodes a granule-localizing domain, wherein the granule-localizing domain is as taught herein and/or preferably:
a granzyme protein or a leader peptide thereof, wherein the granzyme protein may be one or more selected from any known human granzyme (such as granzyme A, granzyme B, granzyme H, granzyme K, and granzyme M, preferably granzyme B);
perforin or a leader peptide thereof;
serglycin or a leader peptide thereof;
granulysin or a leader peptide thereof, and/or
an N-Acetylglucosamine-1 (GlcNAc-1) phosphotransferase binding domain,
and/or wherein the further nucleotide sequence is or comprises preferably:
a nucleotide sequence encoding granzyme A, preferably having at least 40, 50, 60, 70, 80, 85, 90, 95, 96, 97, 98, 99, 99.5, 99.9%, or 100% sequence identity with SEQ ID NO:1, or a nucleotide sequence encoding a leader peptide of granzyme A, preferably having at least 40, 50, 60, 70, 80, 85, 90, 95, 96, 97, 98, 99, 99.5, 99.9%, or 100% sequence identity with SEQ ID NO:2;
a nucleotide sequence encoding granzyme B, preferably having at least 40, 50, 60, 70, 80, 85, 90, 95, 96, 97, 98, 99, 99.5, 99.9%, or 100% sequence identity with SEQ ID NO:3, or a nucleotide sequence encoding a leader peptide of granzyme B, preferably having at least 40, 50, 60, 70, 80, 85, 90, 95, 96, 97, 98, 99, 99.5, 99.9%, or 100% sequence identity with SEQ ID NO:4;
a nucleotide sequence encoding granzyme H, preferably having at least 40, 50, 60, 70, 80, 85, 90, 95, 96, 97, 98, 99, 99.5, 99.9%, or 100% sequence identity with SEQ ID NO:5, or a nucleotide sequence encoding a leader peptide of granzyme H, preferably having at least 40, 50, 60, 70, 80, 85, 90, 95, 96, 97, 98, 99, 99.5, 99.9%, or 100% sequence identity with SEQ ID NO:6;
a nucleotide sequence encoding granzyme K, preferably having at least 40, 50, 60, 70, 80, 85, 90, 95, 96, 97, 98, 99, 99.5, 99.9%, or 100% sequence identity with SEQ ID NO:7, or a nucleotide sequence encoding a leader peptide of granzyme K, preferably having at least 40, 50, 60, 70, 80, 85, 90, 95, 96, 97, 98, 99, 99.5, 99.9%, or 100% sequence identity with SEQ ID NO:8;
a nucleotide sequence encoding granzyme M, preferably having at least 40, 50, 60, 70, 80, 85, 90, 95, 96, 97, 98, 99, 99.5, 99.9%, or 100% sequence identity with SEQ ID NO:9, or a nucleotide sequence encoding a leader peptide of granzyme M, preferably having at least 40, 50, 60, 70, 80, 85, 90, 95, 96, 97, 98, 99, 99.5, 99.9%, or 100% sequence identity with SEQ ID NO:10;
a nucleotide sequence encoding granulysin, preferably having at least 40, 50, 60, 70, 80, 85, 90, 95, 96, 97, 98, 99, 99.5, 99.9%, or 100% sequence identity with SEQ ID NO:11, or a nucleotide sequence encoding leader peptide of granulysin, preferably having at least 40, 50, 60, 70, 80, 85, 90, 95, 96, 97, 98, 99, 99.5, 99.9%, or 100% sequences identity with SEQ ID NO:12;
a nucleotide sequence encoding serglycin, preferably having at least 40, 50, 60, 70, 80, 85, 90, 95, 96, 97, 98, 99, 99.5, 99.9%, or 100% sequence identity with SEQ ID NO:13, or a nucleotide sequence encoding a leader peptide of serglycin, preferably having at least 40, 50, 60, 70, 80, 85, 90, 95, 96, 97, 98, 99, 99.5, 99.9%, or 100% sequence identity with SEQ ID NO:14;
a nucleotide sequence encoding perforin, preferably having at least 40, 50, 60, 70, 80, 85, 90, 95, 96, 97, 98, 99, 99.5, 99.9%, or 100% sequence identity with SEQ ID NO:15, or a nucleotide sequence encoding a leader peptide of perforin, preferably having at least 40, 50, 60, 70, 80, 85, 90, 95, 96, 97, 98, 99, 99.5, 99.9%, or 100% sequence identity with SEQ ID NO:16; and/or
a nucleotide sequence encoding for an N-Acetylglucosamine-1 (GlcNAc-1) phosphotransferase binding domain, preferably having at least 40, 50, 60, 70, 80, 85, 90, 95, 96, 97, 98, 99, 99.5, 99.9%, or 100% sequence identity with SEQ ID NO:17.

In a preferred embodiment, the nucleic acid molecule as taught herein may further comprise a nucleotide sequence encoding a linker molecule.

The linker molecule as taught herein is preferably located between the granule-localizing domain as taught herein and the pro-apoptotic protein as taught herein. In addition or alternatively, the linker molecule is preferably one or more of
ubiquitin;
a peptide consisting of the amino acid Glycine, Asparagine and/or Serine;
a peptide consisting of the amino acid Threonine and/or Alanine, and/or Glycine; and
a peptide consisting of Glycine-Serine residues (GS linker), preferably a GS linker with a length of 1-53 amino acids.

The linker as taught herein may, for example, serve as a way to link a granule-localizing domain and a pro-apoptotic protein, as a means to spatially separate a granule-localizing domain and a pro-apoptotic protein, to provide an additional functionality of a granule-localizing domain and/or a pro-apoptotic protein, or a combination thereof. The length and composition of a linker as taught herein can be varied considerably. The linker as taught herein is preferably a linker peptide, e.g., having a chain length of 1 to 500 amino acid residues (such as 1 to 100, 1 to 50, 6 to 30, 1 to 40, 1 to 20, or less than 30 amino acids or 5 to 10 amino acids). In some embodiments, a linker can be 2, 3, 4, 5, 6, 7, 8 or 9 amino acids in length, or can be 10-20, 20-30, 30-40 or 40-50 amino acids in length.

In addition or alternatively, the nucleic acid molecule as taught herein may comprise a cleavage site for an enzyme, wherein the cleavage site is preferably located between a granule-localizing domain as taught herein and the pro-apoptotic protein as taught herein, wherein the cleavage site is preferably one or more of a cleavage site for a protease, most preferably a cleavage site for a protease selected from the group consisting of a caspase, a cathepsin, an enterokinase, furin, factor Xa, a matrix metalloproteinase, and an aggrecanase, preferably a caspase, most preferably caspase 3. The cleavage site as taught herein is preferably cleaved in the (lytic) granule in the effector cell, following exocytosis by the effector cell, and/or following transfer into the cytosol of the target cell.

In addition or alternatively, the nucleic acid molecule as taught herein may comprise a nucleotide sequence encoding a promoter operatively linked to a nucleotide sequence encoding the granule-localizing domain as taught herein and/or a nucleotide sequence encoding the pro-apoptotic protein as taught herein.

The promoter as taught herein is preferably one or more chosen from: U6 promoter, Hl promoter, CMV (cytomegalovirus) promoter, polyoma virus promoter, adenovirus promoter, fowl pox virus promoter, bovine papilloma virus promoter, avian sarcoma virus promoter, the vaccinia virus 7.5K promoter, simian virus 40 (SV40) promoter, HSV (herpes simplex virus) tk promoter, RSV (Rous sarcoma virus) promoter, elongation factor-1 alpha promoter, metallothionein promoter, beta-actin promoter, human IL-2 gene promoter, human IFN gene promoter, human IL (Interleukin)-4 gene promoter, human lymphotoxin gene promoter, human GM-CSF gene promoter, inducible promoter, tumor cell-specific promoter including TERT (telomerase reverse transcriptase) promoter, PSA (Prostate-specific Antigen) promoter, PSMA (prostate-specific membrane antigen) promoter, CEA (carcinoembryonic antigen) promoter, E2F promoter, or AFP (alpha fetoprotein) promoter, tissue-specific promoter including albumin promoter, actin promoter, and ribosomal protein promoter. In addition or alternatively, chimeric promoters containing sequence elements from two or more different promoters may also be used.

As used herein, a nucleic acid sequence is “operatively linked” when it is placed into a functional relationship with another nucleic acid sequence. For instance, a promoter, or rather a transcription regulatory sequence, is operably linked to a coding sequence if it affects the transcription of the coding sequence. Operably linked means that the DNA sequences being linked are typically contiguous and, where necessary join two or more protein encoding regions, contiguous and in reading frame.

In addition or alternatively, the nucleic acid molecule as taught herein may comprise a nucleotide sequence encoding an enhancer sequence that regulates the transcription and/or translation of the nucleic acid molecule as taught herein. Enhancers are relatively independent in direction and location, but an enhancer from a eukaryotic cell virus such as the SV40 enhancer on the late side of the replication origin (bp 100 to 270) or the CMV early promoter enhancer may be preferably used.

As used herein, the term “promoter” can be understood as a nucleic acid sequence that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. Optionally the term “promoter” includes herein also the 5′ UTR region (5′ Untranslated Region) (e.g., the promoter may herein include one or more parts upstream (5′) of the translation initiation codon of a gene, as this region may have a role in regulating transcription and/or translation).

In one aspect, the current disclosure relates to a pro-apoptotic protein encoded by the nucleotide sequence comprising a BH3 effector domain encoding sequence, an upstream domain encoding sequence, and a downstream domain encoding sequence, as taught herein. In addition or alternatively, the pro-apoptotic protein preferably has at least 40, 50, 60, 70, 80, 85, 90, 95, 96, 97, 98, 99, 99.5, 99.9%, or 100% sequence identity with any one of SEQ ID NO:45-50.

It has been shown that a fusion protein comprising a granule-localizing domain and a (modified) pro-apoptotic protein as taught herein may efficiently localize to a (lytic) granule in an effector cell and/or may efficiently induce apoptosis in a target cell. It is considered that the granule-localizing domain and the pro-apoptotic protein are preferably initially linked to each other following translation, but are separated from each other once localized in the (lytic) granule, following exocytosis, and/or following transfer to the target cell.

In one aspect, the current disclosure relates to a pro-apoptotic construct, preferably a fusion protein and/or a chimeric protein, comprising:
1) a granule-localizing domain, preferably a granule-localizing domain as taught herein.
2) a pro-apoptotic protein comprising a BH3 effector domain, preferably a pro-apoptotic protein comprising a BH3 effector domain as taught herein.

The terms “fusion protein” or “chimeric protein” as used herein relate to proteins created through the joining of two or more nucleotide sequences or genes that originally coded for separate proteins. Typically, translation of the fusion gene results in a single or multiple polypeptides with functional properties derived from each of the original proteins. Some fusion proteins may combine whole peptides and therefore contain all functional domains of the original proteins, whereas other fusion proteins, may combine only portions of coding sequences and therefore do not maintain the original functions of the parental genes that formed them. For example, recombinant fusion proteins are typically created artificially by recombinant DNA technology for use in biological research or therapeutics. Chimeric proteins usually designate hybrid proteins made of polypeptides having different functions or physico-chemical patterns.

The granule-localizing domain in the pro-apoptotic construct as taught herein is preferably:
a granzyme protein or a leader peptide thereof, wherein the granzyme protein may be one or more selected from any known human granzyme (such as granzyme A, granzyme B, granzyme H, granzyme K, and granzyme M, preferably granzyme B);
perforin or a leader peptide thereof;
serglycin or a leader peptide thereof;
granulysin or a leader peptide thereof, and/or
an N-Acetylglucosamine-1 (GlcNAc-1) phosphotransferase binding domain.

In addition or alternatively, the granule-localizing domain in the pro-apoptotic construct as taught herein is preferably:
a granule-localizing domain encoded by a nucleotide sequence encoding granzyme A, preferably having at least 40, 50, 60, 70, 80, 85, 90, 95, 96, 97, 98, 99, 99.5, 99.9%, or 100% sequence identity with SEQ ID NO:1, or a nucleotide sequence encoding a leader peptide of granzyme A, preferably having at least 40, 50, 60, 70, 80, 85, 90, 95, 96, 97, 98, 99, 99.5, 99.9%, or 100% sequence identity with SEQ ID NO:2;
a granule-localizing domain encoded by a nucleotide sequence encoding granzyme B, preferably having at least 40, 50, 60, 70, 80, 85, 90, 95, 96, 97, 98, 99, 99.5, 99.9%, or 100% sequence identity with SEQ ID NO:3, or a nucleotide sequence encoding a leader peptide of granzyme B, preferably having at least 40, 50, 60, 70, 80, 85, 90, 95, 96, 97, 98, 99, 99.5, 99.9%, or 100% sequence identity with SEQ ID NO:4;
a granule-localizing domain encoded by a nucleotide sequence encoding granzyme H, preferably having at least 40, 50, 60, 70, 80, 85, 90, 95, 96, 97, 98, 99, 99.5, 99.9%, or 100% sequence identity with SEQ ID NO:5, or a nucleotide sequence encoding a leader peptide of granzyme H, preferably having at least 40, 50, 60, 70, 80, 85, 90, 95, 96, 97, 98, 99, 99.5, 99.9%, or 100% sequence identity with SEQ ID NO:6;
a granule-localizing domain encoded by a nucleotide sequence encoding granzyme K, preferably having at least 40, 50, 60, 70, 80, 85, 90, 95, 96, 97, 98, 99, 99.5, 99.9%, or 100% sequence identity with SEQ ID NO:7, or a nucleotide sequence encoding a leader peptide of granzyme K, preferably having at least 40, 50, 60, 70, 80, 85, 90, 95, 96, 97, 98, 99, 99.5, 99.9%, or 100% sequence identity with SEQ ID NO:8;
a granule-localizing domain encoded by a nucleotide sequence encoding granzyme M, preferably having at least 40, 50, 60, 70, 80, 85, 90, 95, 96, 97, 98, 99, 99.5, 99.9%, or 100% sequence identity with SEQ ID NO:9, or a nucleotide sequence encoding a leader peptide of granzyme M, preferably having at least 40, 50, 60, 70, 80, 85, 90, 95, 96, 97, 98, 99, 99.5, 99.9%, or 100% sequence identity with SEQ ID NO:10;
a granule-localizing domain encoded by a nucleotide sequence encoding granulysin, preferably having at least 40, 50, 60, 70, 80, 85, 90, 95, 96, 97, 98, 99, 99.5, 99.9%, or 100% sequence identity with SEQ ID NO:11, or a nucleotide sequence encoding leader peptide of granulysin, preferably having at least 40, 50, 60, 70, 80, 85, 90, 95, 96, 97, 98, 99, 99.5, 99.9%, or 100% sequence identity with SEQ ID NO:12;
a granule-localizing domain encoded by a nucleotide sequence encoding serglycin, preferably having at least 40, 50, 60, 70, 80, 85, 90, 95, 96, 97, 98, 99, 99.5, 99.9%, or 100% sequence identity with SEQ ID NO:13, or a nucleotide sequence encoding a leader peptide of serglycin, preferably having at least 40, 50, 60, 70, 80, 85, 90, 95, 96, 97, 98, 99, 99.5, 99.9%, or 100% sequence identity with SEQ ID NO:14;
a granule-localizing domain encoded by a nucleotide sequence encoding perforin, preferably having at least 40, 50, 60, 70, 80, 85, 90, 95, 96, 97, 98, 99, 99.5, 99.9%, or 100% sequence identity with SEQ ID NO:15, or a nucleotide sequence encoding a leader peptide of perforin, preferably having at least 40, 50, 60, 70, 80, 85, 90, 95, 96, 97, 98, 99, 99.5, 99.9%, or 100% sequence identity with SEQ ID NO:16; and/or
a granule-localizing domain encoded by a nucleotide sequence encoding for an N-Acetylglucosamine-1 (GlcNAc-1) phosphotransferase binding domain, preferably having at least 40, 50, 60, 70, 80, 85, 90, 95, 96, 97, 98, 99, 99.5, 99.9%, or 100% sequence identity with SEQ ID NO:17.

The BH3 effector domain in the pro-apoptotic construct as taught herein preferably binds a multidomain BCL-2 family protein and/or a BH3 domain-binding groove of a multidomain BCL-2 family protein, such as a (multidomain) pro-apoptotic BCL-2 family protein and/or a BH3 domain-binding groove of a (multidomain) pro-apoptotic BCL-2 family protein, and/or a (multidomain) pro-survival BCL-2 family protein and/or a BH3 domain-binding groove of a (multidomain) pro-survival BCL-2 family protein, preferably a (multidomain) pro-survival BCL-2 family protein and/or a BH3 domain-binding groove of a (multidomain) pro-survival BCL-2 family protein.

The pro-apoptotic protein comprising a BH3 effector domain in the pro-apoptotic construct as taught herein may be a BH3-only pro-apoptotic protein as taught herein.

In addition or alternatively, the pro-apoptotic protein comprising a BH3 effector domain in the pro-apoptotic construct as taught herein may be a pro-apoptotic protein encoded by the nucleotide sequence comprising a BH3 effector domain encoding sequence, an upstream domain encoding sequence, and a downstream domain encoding sequence, as taught herein.

In addition or alternatively, the pro-apoptotic protein comprising a BH3 effector domain in the pro-apoptotic construct as taught herein may be a pro-apoptotic protein encoded by any one of SEQ ID NO:45, 46, 47, 48, 49, 50.

In a preferred embodiment, the pro-apoptotic construct as taught herein further comprises:
a linker molecule between the granule-localizing domain and the pro-apoptotic protein, preferably a linker molecule as taught herein.
a cleavage site between the granule-localizing domain and the pro-apoptotic protein, preferably a cleavage site as taught herein.

The nucleic acid molecule as taught herein may comprise a nucleotide sequence encoding an epitope tag, preferably one or more chosen from a Hemagglutinin (HA) tag, polyhistidine tag, FLAG tag, AU1 tag, AU5 tag, Myc tag, Glu-Glu tag, E. coli OmpF Linker and mouse Langerin fusion Sequence (OLLAS) tag, T7 tag, V5 tag, VSV-G tag, entity tag (E-Tag), Service VLAN tag (S-Tag), Avi tag, herpes simplex virus (HSV) tag, KT3 tag, TK15 tag, calmodulin-binding protein (CBP) tag, maltose-binding protein tag, beta-galactosidase tag, glutathione S transferase (GST) tag, and thioredoxin tag. A pro-apoptotic protein as taught herein or a pro-apoptotic construct as taught herein may comprise one or more epitope tags as taught herein. The HA-tag may allow for easy visualization of a protein comprising the tag by microscopy, flow cytometry and Western blot.

In addition or alternatively, the nucleic acid molecule as taught herein may comprise a nucleotide sequence encoding a fluorescent protein, preferably green fluorescent protein (GFP), enhanced GFP, and/or mCherry. A pro-apoptotic protein as taught herein or a pro-apoptotic construct as taught herein may comprise one or more fluorescent proteins as taught herein. The HA-tag may allow for easy visualization of a protein comprising the fluorescent protein by a fluorescence detection method such as fluorescence microscopy.

In one aspect, the current disclosure relates to a nucleic acid delivery construct comprising a nucleic acid molecule as taught herein, wherein the nucleic acid delivery construct is preferably chosen from one or more of a plasmid, a recombinant adenovirus, an adeno-associated virus (AAV), a retrovirus, a lentivirus, a herpes simplex virus, and a vaccinia virus, preferably a lentivirus.

In an embodiment, the current disclosure provides for a nucleic acid delivery construct including a nucleic acid molecule as taught herein. The term “nucleic acid delivery construct” as used herein relates to a nucleotide sequence comprising a region (transcribed region), that is transcribed into an RNA molecule (e.g., an Mrna) in a cell, operably linked to suitable regulatory regions (e.g., a promoter). A nucleic acid delivery construct may thus comprise several operably linked sequences, such as a promoter, a 5′ leader sequence comprising, e.g., sequences involved in translation initiation, a (protein) encoding region, splice donor and acceptor sites, intronic and exonic sequences, and a 3′ non-translated sequence (also known as 3′ untranslated sequence or 3′UTR) comprising, e.g., transcription termination sequence sites. A nucleic acid delivery construct may be comprised in a DNA vector or in a viral vector. Introduction of a nucleic acid or nucleic acids may be via transfection or transduction methods depending on what type of nucleic acid or nucleic acids are used. It is understood that depending on what type of nucleic acid delivery construct or constructs are used, the nucleic acid delivery construct may consist of DNA or RNA. For example, when a nucleic acid delivery construct is incorporated in a retroviral or lentiviral vector, the nucleic acid delivery construct is comprised in an RNA vector genome (i.e., the sequence that encodes the nucleic acid delivery construct). Retroviral and lentiviral vectors are well known in the art having an RNA genome that, when entered in a cell, is reverse transcribed into DNA that is subsequently integrated into the host genome. Reverse transcription thus results in the genetic information, i.e., the nucleic acid delivery construct, being transformed from RNA into double-stranded DNA, thereby allowing expression therefrom. Integration is advantageous as it allows proliferation of transduced cells while maintaining the viral vector genome comprising the nucleic acid delivery construct. A nucleic acid delivery construct may also be comprised in a DNA vector, e.g., plasmid DNA. A suitable DNA vector may be a transposon. Suitable transposon systems (e.g., class I or class II based) are well known in the art.

In a preferred embodiment, the nucleic acid delivery construct as taught herein is a lentivirus or a lentiviral vector. Lentiviruses are part of a larger group of retroviruses. Examples of primate lentiviruses suitable as nucleic acid delivery construct include, but are not limited to, human immunodeficiency virus (HIV) and simian immunodeficiency virus (SIV). The non-primate lentivirus group suitable as nucleic acid delivery construct include, but are not limited to, “slow virus” Visna/Maedi virus (VMV) and related caprine arthritis encephalitis virus (CAEV), equine infectious anemia virus (EIAV), feline immunodeficiency virus (FIV), and bovine immunodeficiency virus (BIV). The terms “lentiviral vector” or “lentivirus” as used herein relate to a vector comprising at least one component derivable from lentivirus. Preferably, the component part is responsible for the biological mechanism by which the vector infects the cell, expresses the gene, or is replicated. The lentiviral vector may be a “non-primate” vector, i.e., it may be derived primarily from a virus that does not infect a primate, especially a human.

In an embodiment, the nucleic acid delivery construct as taught herein is a recombinant lentiviral vector or a recombinant lentivirus. The terms “recombinant lentiviral vector” or “recombinant lentivirus” refer to a recombinant lentiviral vector containing sufficient lentiviruses to permit packaging of the RNA genome in the presence of packaging components into viral particles capable of infecting target Infection of target cells may involve reverse transcription and incorporation into the target cell genome. Recombinant lentiviral vectors have non-viral coding sequences that are to be delivered to the target cells by the vector. Recombinant lentiviral vectors cannot make independent replication to produce infectious lentiviral particles within the final target cells. Generally, recombinant lentiviral vectors lack functional gag-pol and/or env genes and/or other genes essential for replication. The recombinant lentiviral vector of the present disclosure may have minimal viral genome. As used herein, the term “minimal viral genome” provides the functionality required for infection and transduces the nucleotide sequence of interest to the target host cell. It means that the viral vector has been manipulated to remove nonessential elements and to retain essential elements for delivery. In one embodiment of the disclosure, the lentiviral vector is a self-inactivating vector.

In one aspect, the current disclosure relates to a delivery system comprising a nucleic acid delivery construct as taught herein.

In one aspect, the current disclosure relates to a delivery system comprising a pro-apoptotic protein as taught herein.

A suitable delivery system that can be used instead of the granule-localizing domain to target the pro-apoptotic protein into a target cell has been considered. For example, the pro-apoptotic protein can be delivered as part of a delivery system that is functionalized with targeting ligands that preferentially bind to cancer cells, such as tumor-associated antigens, and preferably do not target other cells or does not induce toxicity (e.g., as described in Haen et al., Nat. Rev. Clin. Oncol. 2020 October; 17(10):595-610). In an embodiment, the present disclosure relates to NOXA or modified NOXA (i.e., substituted BH3-effector domain), preferably NOXA(BIM) that is not coupled to a granule-localizing domain. In an embodiment, the present disclosure relates to NOXA or modified NOXA (i.e., substituted BH3-effector domain), preferably NOXA(BIM), preferably incorporated into a delivery system (allowing targeting into a target cell).

The delivery system as taught herein is preferably one or more chosen from a synthetic nanoparticle, a lipid nanoparticle, and/or a polymeric nanoparticle. In addition or alternatively, the delivery system may be one or more of a nanocrystal, polymer-drug conjugate, polymeric micelle, dendrimer, protein-based nanoparticle, silver nanoparticle, ceramic nanoparticle, carbon-based nanoparticle, hydrogel nanoparticle, and superparamagnetic nanoparticle. The delivery system may also be an isolated natural-occurring particle, such as a (lytic) granule and/or a (secretory) vesicle. Preferably, the delivery system provides intracellular delivery or intracellular uptake of a nucleic acid molecule, a pro-apoptotic protein, and/or a pro-apoptotic construct as taught herein. The delivery system may be delivered to a subject as part of a medical therapy, preferably a cancer therapy, as taught herein. The route of administration of delivery system as taught herein may be any route that provides a therapeutic effect, preferably an anti-cancer effect. The route of administration may, for example, include oral, topical, transdermal, transcutaneous, intramuscular, intraperitoneal, intravenous, subcutaneous injection. The composition as taught herein may be applied systemically (e.g., by oral intake, by systemic injection) or locally (e.g., local injection, topical). For example, injection of the delivery system in vicinity of a tumor may be preferred for solid tumors, whereas systemic injection of the delivery system may be preferred for hematological tumors. In a preferred embodiment, the delivery system as taught herein is delivered locally (e.g., transcutaneous delivery or via an open wound such as in a surgical setting). In addition or alternatively, the delivery system as taught herein is preferably delivered systemically (e.g., by intravenous delivery such as intravenous injection). In an embodiment, the delivery system as taught herein is functionalized with targeting ligands that preferentially bind to cancer cells, such as tumor-associated antigens. The skilled person is familiar with the tumor-associated antigens that may be targeted using a functionalized delivery system (e.g., Haen et al., Nat. Rev. Clin. Oncol. 2020 October; 17(10):595-610).

In an embodiment, the delivery system as taught herein:
is a (lytic) granule;
is a (secretory) vesicle;
has a diameter of between 0.1 to 1.3 μm; and/or
is a membrane-bound organelle and/or comprise a lipid bilayer.

In one aspect, the current disclosure relates to a cell, preferably an immune cell, more preferably an immune cell that is a cytotoxic lymphocyte (e.g., cytotoxic T cell or NK cell), most preferably an immune cell that is a chimeric antigen receptor T cell or chimeric antigen receptor NK cell, wherein the (immune) cell:
comprises a nucleic acid molecule as taught herein;
expresses a (BH3-only) pro-apoptotic protein encoded by a nucleic acid as taught herein;
expresses a (BH3-only) pro-apoptotic protein as taught herein;
expresses a pro-apoptotic construct as taught herein;
comprises a nucleic acid delivery construct as taught herein;
comprises a delivery system as taught herein;
has an immune receptor, preferably engineered immune receptor, most preferably chimeric antigen receptor;
induces apoptosis of a target cell as taught herein, wherein the target cell is preferably a cancer cell as taught herein, and/or wherein the apoptosis is preferably mediated by inactivating one or more pro-survival BCL-2 family proteins, preferably one or more pro-survival BCL-2 family proteins, most preferably one or more of BCL-2, BCL-XL, BCL-W, MCL-1, BFL-1, and BCL-B; and/or
has a granzyme-perforin pathway.

A cell comprising a nucleic acid molecule as taught herein, expressing a (BH3-only) pro-apoptotic protein encoded by a nucleic acid as taught herein, and/or expressing a (BH3-only) pro-apoptotic protein as taught herein may be referred to herein as a “transduced cell” or a “transfected cell.” “Transduced cells” typically relate to cells that have been infected with a viral vector such as one of the viral nucleic acid delivery constructs as taught herein. For example, a retroviral vector as taught herein may be used, such as described herein, but other suitable viral vectors such as described herein may also be contemplated, such as a lentivirus as taught herein. “Transfected cells” relate to cells wherein a non-viral nucleic acid delivery construct has been introduced, e.g., introduced into the cell by a non-viral method, such as transfection or electroporation of DNA vectors. DNA vectors that may be used are transposon vectors. Transfected cells may be “stably transfected cells” or “transiently transfected cells.” Transfection refers to non-viral methods to transfer DNA (or RNA) to cells such that a gene is expressed. Transfection methods are widely known in the art, such as calcium phosphate transfection, (polyethylene glycol) PEG transfection, nanoparticle transfection, and liposomal or lipoplex transfection of nucleic acid molecules. Such a transfection may be transient but may also be a stable transfection wherein cells can be selected that have the nucleic acid delivery constructs integrated in their genome.

The (transfected and/or transduced) cell as taught herein is preferably a human cell or an animal (preferably a mammal) cell. In addition or alternatively, the (transfected and/or transduced) cell as taught herein is preferably an immune cell. In a preferred embodiment, the (transfected and/or transduced) cell is an effector immune cell in cancer immunotherapy such as a cytotoxic T cell or an NK cell, more preferably an engineered cytotoxic T cell or NK cell such as a chimeric antigen receptor (CAR) T or NK cell.

An “immune cell” according to the present disclosure may be any cell belonging to the immune system, preferably chosen from a lymphocyte, granulocyte, myeloid cell, T cell (e.g., T helper cell, T helper 17 cell, follicular helper T cell, cytotoxic T cell, gamma delta T cell), monocyte, macrophage, NK cell, basophil, dendritic cell (e.g., myeloid dendritic cell, plasmacytoid dendritic cell), neutrophil, eosinophil, basophil, mast cell, B cell, or plasma cell, among others. An “immune cell” according to the present disclosure may also be an engineered immune cell as taught herein. In a preferred embodiment, the immune cell according to the present disclosure is an engineered cytotoxic T cell or NK cell such as a chimeric antigen receptor (CAR) T or CAR NK cell.

The term “engineered cell” as used herein refers to a cell hat has been engineered, e.g., by the introduction of an exogenous nucleic acid sequence or specific alteration of an endogenous gene sequence (e.g., by introducing a nucleic acid molecule and/or a nucleic acid delivery construct as taught herein). An exogenous nucleic acid sequence that is introduced may comprise a wildtype sequence of any species that may be modified. An engineered cell may comprise genetic modifications such as one or more mutations, insertions and/or deletions in an endogenous gene and/or insertion of an exogenous nucleic acid (e.g., a nucleic acid delivery construct) in the genome. An engineered cell may refer to a cell in isolation or in culture. In a preferred embodiment, the engineered cell is an immune cell that has been engineered to bind to and/or mediate the killing of a selected target cell population such as a cancer cell. For example, this may be an immune cell such as a T cell, preferably a cytotoxic T cell, engineered so as to recognize a specific molecule on a target cell such as a cancer cell, a CAR cell. A “CAR cell” is a cell expressing a CAR, i.e., a recombinant receptor that combines the specificity of an antigen-specific antibody with the T-cell's activating functions (as reviewed Shi et al., Mol. Cancer. 2014 Sep. 21; 13:219). A CAR may be a fusion molecule between an antibody and a trans-membrane domain allowing expression of an antibody at the cell surface of an immune cell as well as signaling into the cell.

In an embodiment of the current disclosure, a (transfected and/or transduced) as taught herein cell may comprise a selectable marker. A selectable marker may be defined as any nucleic acid sequence and/or amino acid sequence in addition to the nucleic acid molecule as taught herein that allows a cell that is provided therewith to be selected. For example, a selectable marker may be a neomycin or puromycin resistance gene. Selection of a cell to which a nucleic acid delivery construct has been transferred may than be performed by incubating in the presence of neomycin or puromycin. Other selectable markers may be, for example, any one of green, red and yellow fluorescent proteins. Selection may then be performed by using, e.g., fluorescence-activated cell sorting (FACS).

In one aspect, the current disclosure relates to the use of a nucleic acid molecule as taught herein in a medical therapy.

In an embodiment, the nucleic acid molecule, pro-apoptotic protein or pro-apoptotic construct as taught herein, preferably comprising NOXA, more preferably comprising NOXA(BIM), is for use in a therapy, more particularly, for use in the treatment of a cancer as disclosed herein. In an embodiment, the cancer is insensitive to MCL-1 inhibition and/or wildtype NOXA. The sensitivity of a cancer or cancer cell type to MCL-1 inhibition and/or wildtype NOXA can be determined by subjecting cancer cells to NOXA constructs targeting MCL-1 (as referred to as NOXA WT or “NOXA” in the Examples) or synthetic NOXA (having same sequence as wildtype NOXA) and measuring specific apoptosis by staining for apoptosis (e.g., with DiOC6 and/or TO-PRO-3) followed by analysis by flow cytometry, as according to the Examples. Insensitivity to MCL-1 inhibition and/or wildtype NOXA is preferably defined by less than 25%, more preferably less than 10%, even more preferably less than 5% improvement in specific apoptosis after 24 hours treatment, as compared to the appropriate control (e.g., no treatment or treatment with NOXA targeting nothing (NOXA(3E), “Inoxa”) as disclosed in the Examples.

In one aspect, the current disclosure relates to the use of a (BH3-only) pro-apoptotic protein as taught herein in a medical therapy.

In one aspect, the current disclosure relates to the use of a pro-apoptotic construct as taught herein, preferably a fusion protein as taught herein, in a medical therapy.

In one aspect, the current disclosure relates to the use of a nucleic acid delivery construct as taught herein in a medical therapy.

In one aspect, the current disclosure relates to the use of an immune cell as taught herein, preferably a transduced cell, a transfected cell, and/or a cell comprising a nucleic acid delivery construct as taught herein, in a medical therapy.

In one aspect, the current disclosure relates to the use of a delivery system as taught herein in a medical therapy.

The medical therapy as taught herein is preferably cancer therapy, more preferably cancer immunotherapy, wherein the cancer preferably is one or more of melanoma, liver cancer, breast cancer, colorectal cancer, lung cancer, prostate cancer, multiple myeloma, lymphoma or leukemia.

In addition or alternatively, the medical therapy as taught herein may relate to the prevention and/or treatment of, among others:
a hyperproliferative or differentiative disorder, which may, e.g., be fibrosis or hyperplasia, such as pulmonary fibrosis or hyperplasia (e.g., benign prostatic hyperplasia), cardiac fibrosis, or liver fibrosis;
an infectious condition, such as a bacterial, viral or fungal infection, preferably a viral infection, e.g., selected from human immunodeficiency virus (HIV) infection, hepatitis C virus (HCV) infection, hepatitis B infections (HBV), or cytomegalovirus (CMV) infection;
an inflammatory condition, such as a prostatitis, vernal keratoconjunctivitis, atherosclerosis, or idiopathic pulmonary pneumonia;
an autoimmune condition, such as systemic lupus erythematosus (SLE), multiple sclerosis (MS), pemphigus vulgaris (PV), or myasthenia gravis; and/or
cancer.

The cancer (targeted by the medical treatment) as disclosed herein includes, but is not limited to biliary tract cancer, brain cancer (e.g., including glioblastomas and medulloblastomas), breast cancer (e.g., including inflammatory breast cancer), cervical cancer, choriocarcinoma, colon cancer, endometrial cancer, esophageal cancer, gastric cancer, hematological neoplasms (e.g., including acute lymphocytic and myelogenous leukemia), multiple myeloma, AIDS associated leukemias and adult T-cell leukemia lymphoma, intraepithelial neoplasms (e.g., including Bowen's disease and Paget's disease), liver cancer (hepatocarcinoma), lung cancer, lymphomas (e.g., including Hodgkin's disease and lymphocytic lymphomas), neuroblastomas, oral cancer (e.g., including squamous cell carcinoma), ovarian cancer (e.g., including those arising from epithelial cells, stromal cells, germ cells and mesenchymal cells), pancreas cancer, prostate cancer, rectal cancer, sarcomas (e.g., including leiomyosarcoma, rhabdomyosarcoma, liposarcoma, fibrosarcoma and osteosarcoma), skin cancer (e.g., including melanoma, Kaposi's sarcoma, basocellular cancer and squamous cell cancer), testicular cancer (e.g., including germinal tumors, seminoma, non-seminoma [teratomas, choriocarcinomas]), stromal tumors and germ cell tumors, thyroid cancer (e.g., including thyroid adenocarcinoma and medullar carcinoma), and renal cancer (e.g., including adenocarcinoma and Wilms tumor).

In an embodiment, the cancer is refractory to one or more prior treatments, and/or the cancer has relapsed after one or more prior treatments.

In one aspect, the current disclosure relates to a method to produce a transfected and/or transduced cell as taught herein, e.g., comprising the step of:
i. providing cells, preferably human immune cells, more preferably human NK cells and/or human T cells, even more preferably engineered human NK cells and/or engineered human T cells, most preferably human CAR-engineered T or human CAR-engineered NK cells;
ii. providing a nucleic acid molecule as taught herein, wherein the nucleic acid molecule is preferably comprised in a nucleic acid delivery construct as taught herein;
iii. introducing the nucleic acid molecule provided in step ii) into the cells provided in step i) to thereby obtain cells expressing a translated molecule, e.g., a (pro-apoptotic) amino acid sequence, a (pro-apoptotic) peptide, a (pro-apoptotic) protein, a (pro-apoptotic) construct, and/or a (pro-apoptotic) fusion protein) encoded the nucleic acid molecule provided in step ii); and
iv. optionally selecting and/or separating the cells expressing the translated molecule obtained in step iii), e.g., by selecting and/or separating cells based on their expression of a selectable marker as disclosed herein.

In one aspect, the current disclosure relates to a method to produce a (pro-apoptotic) amino acid sequence, a (pro-apoptotic) peptide, a (pro-apoptotic) protein, a (pro-apoptotic) fusion protein, preferably encoded by a nucleic acid molecule as taught herein, e.g., comprising the step of
i. providing an expression system;
ii. providing a nucleic acid molecule as taught herein;
iii. introducing the nucleic acid molecule provided in step ii) into the expression system provided in step i) to thereby provide an expression system expressing a (pro-apoptotic) amino acid sequence, a (pro-apoptotic) peptide, a (pro-apoptotic) protein, and/or a (pro-apoptotic) fusion protein encoded by the nucleic acid molecule provided in step ii). The expression system expressing the (pro-apoptotic) amino acid sequence, the (pro-apoptotic) peptide, the (pro-apoptotic) protein, and/or the (pro-apoptotic) fusion protein encoded by the nucleic acid molecule may optionally be selected and/or separated, e.g., by selecting and/or separating cells based on their expression of a selectable marker as disclosed herein; and
iv. obtaining the (pro-apoptotic) amino acid sequence, the (pro-apoptotic) peptide, the (pro-apoptotic) protein, and/or the (pro-apoptotic) fusion protein expressed by the expression system in step iii).

An “expression system” as used herein relates to a system that generates an amino acid sequence, peptide, and/or protein achieved by the manipulation of gene expression in an organism such that it expresses large amounts of a recombinant gene. This includes the transcription of the recombinant DNA to messenger RNA (Mrna), the translation of Mrna into polypeptide chains, that are ultimately folded into a functional amino acid sequence, peptide, and/or protein and that may be targeted to specific subcellular or extracellular locations. Preferably the expression system includes one derived from bacteria (e.g., Escherichia coli, Corynebacterium, Pseudomonas fluorescens) yeast, baculovirus/insect, or a mammalian cell (e.g., Chinese Hamster ovary, human embryonic kidney cell).

In the present disclosure, the nucleic acid molecule as taught herein, the pro-apoptotic protein as taught herein, the pro-apoptotic construct as taught herein, the nucleic acid delivery construct as taught herein and/or the immune cell as taught herein may be combined with or comprise a granzyme-perforin pathway (or immune cell comprising the same) and/or a pore forming protein preferably perforin (or nucleotide sequence encoding the same).

In various of the embodiments, a (pro-apoptotic) protein as disclosed herein or granule localizing domain as disclosed herein may have “conservative substitutions” in one or more amino acid positions. The term “conservative substitution” relates herein to a situation wherein a residue is replaced by another of the same general type. It is well within grasp of the skilled person that a conservative substitution can lead to a protein with similar biological activity. Furthermore, the skilled person also knows that proteins having a different amino acid sequence can have the same activity. It is common general knowledge that it is often possible to substitute a certain amino acid by another one, without loss of activity of the protein. For example, the following amino acids can often be exchanged for one another:
Ala, Ser, Thr, Gly (small aliphatic, nonpolar or slightly polar residues);
Asp, Asn, Glu, Gln (polar, negatively charged residues and their amides);
His, Arg, Lys (polar, positively charged residues);
Met, Leu, Ile, Val (Cys) (large aliphatic, nonpolar residues); and
Phe, Ty, Trp (large aromatic residues).

In making conservative substitutions, the hydropathic index of amino acids may be considered (See, e.g., Kyte et al., J. Mol. Biol. 157, 105-132, 1982). It is known in the art that certain amino acids may be substituted by other amino acids having a similar hydropathic index or score and still result in a protein having similar biological activity. In making such changes, the substitution of amino acids whose hydropathic indices are within ±2 is preferred, those that are within ±1 are more preferred, and those within ±0.5 are even more preferred. Similarly, select amino acids may be substituted by other amino acids having a similar hydrophilicity, as set forth in U.S. Pat. No. 4,554,101 (herein incorporated by reference in its entirety). In making such changes, as with the hydropathic indices, the substitution of amino acids whose hydrophilicity indices are within +2 is preferred, those that are within ±1 are more preferred, and those within +0.5 are even more preferred.

In various embodiments, a nucleotide sequence as disclosed herein may have on or more variations that encode for the same protein or a protein with similar biological activity. It is well within grasp of the skilled person that polynucleotides having a different nucleotide sequence can encode the same protein. For example, it is common general knowledge that codon usage may vary among polynucleotides encoding the same polypeptide. Specifically, polypeptide-encoding polynucleotides use a triplet code, i.e., a codon code, wherein three bases make up a codon. Because there are four bases (A, C, T, G) possible for each of the three positions in the codon, 64 different codons are possible. However, there are only 20 different amino acids. The overabundance in the number of codons underlies the fact that most amino acids are encoded by more than one codon code.

In this document and in its claims, the verb “to comprise” and its conjugations is used in its non-limiting sense to mean that items following the word are included, but items not specifically mentioned are not excluded. It encompasses the verbs “consisting essentially of” as well as “consisting of.”

As used herein, the singular forms “a,” “an” and “the” include plural referents unless the context clearly dictates otherwise. For example, a method for isolating “a” DNA molecule includes isolating a plurality of molecules (e.g., 10's, 100's, 1000's, 10's of thousands, 100's of thousands, millions, or more molecules).

The term “sequence identity” or “sequence similarity” as used herein refer to a situation where an amino acid or a nucleic acid sequence has sequence identity or sequence similarity with another reference amino acid or nucleic acid sequence. “Sequence identity” or “sequence similarity” can be determined by alignment of two polypeptides or two nucleotide sequences using global or local alignment algorithms. Sequences may then be referred to as “substantially identical” or “essentially similar” when they (when optimally aligned by, for example, the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or the EMBOSS package (EMBOSS: The European Molecular Biology Open Software Suite, Rice et al., 2000, Trends Genet. 16: 276-277), preferably version 5.0.0 or later (using the program “Needle”). For example, the output of Needle in the EMBOSS package labeled “longest identity” (obtained using the -nobrief option) is used as the percent identity and is calculated as follows:

(Identical Residues×100)/(Length of Alignment−Total Number of Gaps in Alignment).

The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1).

### LIST OF SEQUENCES

TABLE 1
List of Sequences

| SEQ ID | | | |
|---|---|---|---|
| NO: | Unit | Molecule | Sequence |
| 1 | NT | Granzyme A | atgaggaactcctatagatttctggcatcctctctctcagttgtc |
| | | | gtttctctcctgctaattcctgaagatgtctgtgaaaaaattatt |
| | | | ggaggaaatgaagtaactcctcattcaagaccctacatggtccta |
| | | | cttagtcttgacagaaaaaccatctgtgctggggctttgattgca |
| | | | aaagactgggtgttgactgcagctcactgtaacttgaacaaaagg |
| | | | tcccaggtcattcttggggctcactcaataaccagggaagagcca |
| | | | acaaaacagataatgcttgttaagaaagagtttccctatccatgc |
| | | | tatgacccagccacacgcgaaggtgaccttaaacttttacagctg |
| | | | acggaaaaagcaaaaattaacaaatatgtgactatccttcatcta |
| | | | cctaaaaagggggatgatgtgaaaccaggaaccatgtgccaagtt |
| | | | gcagggtggggcaggactcacaatagtgcatcttggtccgatact |
| | | | ctgagagaagtcaatatcaccatcatagacagaaaagtctgcaat |
| | | | gatcgaaatcactataattttaaccctgtgattggaatgaatatg |
| | | | gtttgtgctggaagcctccgaggtggaagagactcgtgcaatgga |
| | | | gattctggaagccctttgttgtgcgagggtgttttccgaggggtc |
| | | | acttcctttggccttgaaaataaatgcggagaccctcgtgggcct |
| | | | ggtgtctatattcttctctcaaagaaacacctcaactggataatt |
| | | | atgactatcaagggagcagtttaa |
| 2 | NT | Granzyme A | Atgaggaactcctatagatttctggcatcctctctctcagttgtc |
| | | leader peptide | gtttctctcctgctaattcctgaagatgtctgtgaaaaaattatt |
| 3 | NT | Granzyme B | atgcagccaatactgctcctgttggccttcctcctcctcccgcgg |
| | | | gcagatgcaggcgaaataattggtgggcatgaagcgaaaccccac |
| | | | tcacgcccatatatggcttatttgatgatatgggaccagaaatct |
| | | | cttaaacgatgcggtggatttctcatccgcgatgattttgtgctc |
| | | | actgcagctcattgttgggggtcaagtatcaatgtaacactcggt |
| | | | gctcacaatatcaaagagcaagagcccacgcaacagtttataccc |
| | | | gttaaacggccgatccctcatcctgcctataaccccaagaacttc |
| | | | tctaacgacattatgctccttcaattggagcggaaggctaagcga |
| | | | acaagagcggttcagcccctccggctgccttccaacaaagctcaa |
| | | | gtaaaaccgggccagacgtgctccgtcgcagggtggggtcaaact |
| | | | gccccactcggcaagcactcccacacgcttcaggaagtgaagatg |
| | | | acagtgcaggaagaccgcaagtgcgaaagcgaccttcggcactat |
| | | | tacgactctacaatcgaattgtgcgtgggagatcctgagatcaag |
| | | | aagacctccttcaagggggatagtgggggtccactggtttgcaac |
| | | | aaggtagctcagggtatagtgtcttatggacgcaataacgggatg |
| | | | ccccctagagcttgcaccaaagtgtcatccttcgccattggatta |
| | | | aaaagacaatgaagcggtac |
| 4 | NT | Granzyme B | atgcagccaatactgctcctgttggccttcctcctcctcccgcgg |
| | | leader peptide | gcagatgcaggcgaaataatt |
| 5 | NT | Granzyme H | Atgcagccattcctcctcctgttggcctttcttctgacccctggg |
| | | | gctgggacagaggagatcatcgggggccatgaggccaagccccac |
| | | | tcccgcccctacatggcctttgttcagtttctgcaagagaagagt |
| | | | cggaagaggtgtggcggcatcctagtgagaaaggactttgtgctg |
| | | | acagctgctcactgccagggaagctccataaatgtcaccttgggg |
| | | | gcccacaatatcaaggaacaggagcggacccagcagtttatccct |
| | | | gtgaaaagacccatcccccatccagcctataatcctaagaacttc |
| | | | tccaacgacatcatgctactgcagctggagagaaaggccaagtgg |
| | | | accacagctgtgcggcctctcaggctacctagcagcaaggcccag |
| | | | gtgaagccagggcagctgtgcagtgtggctggctggggttatgtc |
| | | | tcaatgagcactttagcaaccacactgcaggaagtgttgctgaca |
| | | | gtgcagaaggactgccagtgtgaacgtctcttccatggcaattac |
| | | | agcagagccactgagatttgtgtgggggatccaaagaagacacag |
| | | | accggtttcaagggggactccggggggcccctcgtgtgtaaggac |
| | | | gtagcccaaggtattctctcctatggaaataaaaaagggacacct |
| | | | ccaggagtctacatcaaggtctcacacttcctgccctggataaag |
| | | | agaacaatgaagcgcctctaa |
| 6 | NT | Granzyme H | atgcagccattcctcctcctgttggcctttcttctgacccctggg |
| | | leader peptide | gctgggacagaggagatcatc |
| 7 | NT | Granzyme K | Atgactaagttttcttccttttctctgtttttcctaatagttggg |
| | | | gcttatatgactcatgtgtgtttcaatatggaaattattggaggg |
| | | | aaagaagtgtcacctcattccaggccatttatggcctccatccag |
| | | | tatggcggacatcacgtttgtggaggtgttctgattgatccacag |
| | | | tgggtgctgacagcagcccactgccaatatcggtttaccaaaggc |
| | | | cagtctcccactgtggttttaggcgcacactctctctcaaagaat |
| | | | gaggcctccaaacaaacactggagatcaaaaaatttataccattc |
| | | | tcaagagttacatcagatcctcaatcaaatgatatcatgctggtt |
| | | | aagcttcaaacagccgcaaaactcaataaacatgtcaagatgctc |
| | | | cacataagatccaaaacctctcttagatctggaaccaaatgcaag |
| | | | gttactggctggggagccaccgatccagattcattaagaccttct |
| | | | gacaccctgcgagaagtcactgttactgtcctaagtcgaaaactt |
| | | | tgcaacagccaaagttactacaacggcgacccttttatcaccaaa |
| | | | gacatggtctgtgcaggagatgccaaaggccagaaggattcctgt |
| | | | aagggtgactcagggggccccttgatctgtaaaggtgtcttccac |
| | | | gctatagtctctggaggtcatgaatgtggtgttgccacaaagcct |
| | | | ggaatctacaccctgttaaccaagaaataccagacttggatcaaa |
| | | | agcaaccttgtcccgcctcatacaaattaa |
| 8 | NT | Granzyme K | Atgactaagttttcttccttttctctgtttttcctaatagttggg |
| | | leader peptide | gcttatatgactcatgtgtgtttcaatatggaaattatt |
| 9 | NT | Granzyme M | Atggaggcctgcgtgtcttcactgctggtgctggccctgggggcc |
| | | | ctgtcagtaggcagctcctttgggacccagatcatcgggggccgg |
| | | | gaggtgatcccccactcgcgcccgtacatggcctcactgcagaga |
| | | | aatggctcccacctgtgcgggggtgtcctggtgcacccaaagtgg |
| | | | gtgctgacggctgcccactgcctggcccagcggatggcccagctg |
| | | | aggctggtgctggggctccacaccctggacagccccggtctcacc |
| | | | ttccacatcaaggcagccatccagcaccctcgctacaagcccgtc |
| | | | cctgccctggagaacgacctcgcgctgcttcagctggacgggaaa |
| | | | gtgaagcccagccggaccatccggccgttggccctgcccagtaag |
| | | | cgccaggtggtggcagcagggactcggtgcagcatggccggctgg |
| | | | gggctgacccaccagggcgggcgcctgtcccgggtgctgcgggag |
| | | | ctggacctccaagtgctggacacccgcatgtgtaacaacagccgc |
| | | | ttctggaacggcagcctctcccccagcatggtctgcctggcggcc |
| | | | gactccaaggaccaggctccctgcaagggtgactcgggcgggccc |
| | | | ctggtgtgtggcaaaggccgggtgttggccggagtcctgtccttc |
| | | | agctccagggtctgcactgacatcttcaagcctcccgtggccacc |
| | | | gctgtggcgccttacgtgtcctggatcaggaaggtcaccggccga |
| | | | tcggcctga |
| 10 | NT | Granzyme M | Atggaggcctgcgtgtcttcactgctggtgctggccctgggggcc |
| | | leader peptide | ctgtcagtaggcagctcctttgggacccagatcatc |
| 11 | NT | Granulysin | atggctacctgggccctcctgctccttgcagccatgctcctgggc |
| | | | aacccaggtctggtcttctctcgtctgagccctgagtactacgac |
| | | | ctggcaagagcccacctgcgtgatgaggagaaatcctgcccgtgc |
| | | | ctggcccaggagggcccccagggtgacctgttgaccaaaacacag |
| | | | gagctgggccgtgactacaggacctgtctgacgatagtccaaaaa |
| | | | ctgaagaagatggtggataagcccacccagagaagtgtttccaat |
| | | | gctgcgacccgggtgtgtaggacggggaggtcacgatggcgcgac |
| | | | gtctgcagaaatttcatgaggaggtatcagtctagagttacccag |
| | | | ggcctcgtggccggagaaactgcccagcagatctgtgaggacctc |
| | | | aggttgtgtataccttctacaggtcccctctga |
| 12 | NT | Granulysin | atggctacctgggccctcctgctccttgcagccatgctcctgggc |
| | | leader peptide | aacccaggtctggtcttctct |
| 13 | NT | Serglycin | Atgatgcagaagctactcaaatgcagtcggcttgtcctggctctt |
| | | | gccctcatcctggttctggaatcctcagttcaaggttatcctacg |
| | | | cagagagccaggtaccaatgggtgcgctgcaatccagacagtaat |
| | | | tctgcaaactgccttgaagaaaaaggaccaatgttcgaactactt |
| | | | ccaggtgaatccaacaagatcccccgtctgaggactgaccttttt |
| | | | ccaaagacgagaatccaggacttgaatcgtatcttcccactttct |
| | | | gaggactactctggatcaggcttcggctccggctccggctctgga |
| | | | tcaggatctgggagtggcttcctaacggaaatggaacaggattac |
| | | | caactagtagacgaaagtgatgctttccatgacaaccttaggtct |
| | | | cttgacaggaatctgccctcagacagccaggacttgggtcaacat |
| | | | ggattagaagaggattttatgttataa |
| 14 | NT | Serglycin | Atgatgcagaagctactcaaatgcagtcggcttgtcctggctctt |
| | | leader peptide | gccctcatcctggttctggaatcctcagttcaaggt |
| 15 | NT | Perforin | atggcagcccgtctgctcctcctgggcatccttctcctgctgctg |
| | | | cccctgcccgtccctgccccgtgccacacagccgcacgctcagag |
| | | | tgcaagcgcagccacaagttcgtgcctggtgcatggctggccggg |
| | | | gagggtgtggacgtgaccagcctccgccgctcgggctccttccca |
| | | | gtggacacacaaaggttcctgcggcccgacggcacctgcaccctc |
| | | | tgtgaaaatgccctacaggagggcaccctccagcgcctgcctctg |
| | | | gcgctcaccaactggcgggcccagggctctggctgccagcgccat |
| | | | gtaaccagggccaaagtcagctccactgaagctgtggcccgggat |
| | | | gcggctcgtagcatccgcaacgactggaaggtcgggctggacgtg |
| | | | actcctaagcccaccagcaatgtgcatgtgtctgtggccggctca |
| | | | cactcacaggcagccaactttgcagcccagaagacccaccaggac |
| | | | cagtacagcttcagcactgacacggtggagtgccgcttctacagt |
| | | | ttccatgtggtacacactcccccgctgcaccctgacttcaagagg |
| | | | gccctcggggacctgccccaccacttcaacgcctccacccagccc |
| | | | gcctacctcaggcttatctccaactacggcacccacttcatccgg |
| | | | gctgtggagctgggtggccgcatatcggccctcactgccctgcgc |
| | | | acctgcgagctggccctggaagggctcacggacaacgaggtggag |
| | | | gactgcctgactgtcgaggcccaggtcaacataggcatccacggc |
| | | | agcatctctgccgaagccaaggcctgtgaggagaagaagaagaag |
| | | | cacaagatgacggcctccttccaccaaacctaccgggagcgccac |
| | | | tcggaagtggttggcggccatcacacctccattaacgacctgctg |
| | | | ttcgggatccaggccgggcccgagcagtactcagcctgggtaaac |
| | | | tccgtgcccggcagccctggcctggtggactacaccctggaaccc |
| | | | ctgcacgtgctgctggacagccaggacccgcggcgggaggcactg |
| | | | aggagggccctgagtcagtacctgacggacagggctcgctggagg |
| | | | gactgcagccggccgtgcccaccagggcggcagaagagcccccga |
| | | | gacccatgccagtgtgtgtgccatggctcagcggtcaccacccag |
| | | | gactgctgccctcggcagaggggcctggcccagctggaggtgacc |
| | | | ttcatccaagcatggagcctgtggggggactggttcactgccacg |
| | | | gatgcctatgtgaagctcttctttggtggccaggagctgaggacg |
| | | | agcaccgtgtgggacaataacaaccccatctggtcagtgcggctg |
| | | | gattttggggatgtgctcctggccacaggggggcccctgaggttg |
| | | | caggtctgggatcaggactctggcagggacgatgacctccttggc |
| | | | acctgtgatcaggctcccaagtctggttcccatgaggtgagatgc |
| | | | aacctgaatcatggccacctaaaattccgctatcatgccaggtgc |
| | | | ttgccccacctgggaggaggcacctgcctggactatgtcccccaa |
| | | | atgcttctgggggagcctccaggaaaccggagtggggccgtgtgg |
| | | | tga |
| 16 | DNA | Perforin | atggcagcccgtctgctcctcctgggcatccttctcctgctgctg |
| | | leader peptide | cccctgcccgtccctgcc |
| 17 | DNA | GlcNAc-1 | Acagctgctcactgttggggaagctccataaatgtcaccttgggg |
| | | phosphotransferase | gcccacaatatcaaagaacaggagccgacccagcagtttatccct |
| | | binding domain | gtgaaaagacccatcccccatccagcctataatcctaagaacttc |
| | | | tccaacgacatcatgctactgcagctggagagaaagg |
| 18 | DNA | BH3 domain | gaactggaagtggaatgtgccacacagctgcggagattcggcgac |
| | | NOXA | aagctgaacttccgg |
| 19 | DNA | BH3 domain | tcgtccgccgcgcagctcaccgccgcccggctcaaggcgctaggc |
| | | HRK | gacgagctgcaccag |
| 20 | DNA | BH3 domain | ctctgggcagcacagcgctatggccgcgagctccggaggatgagt |
| | | BAD | gacgagtttgtggac |
| 21 | DNA | BH3 domain | gacatcatccggaatattgccaggcacctcgcccaggtcggggac |
| | | BID | agcatggaccgtagc |
| 22 | DNA | BH3 domain | atgcgcccagagatatggatcgcccaagagttgcggcgtatcgga |
| | | BIM | gacgagtttaacgct |
| 23 | DNA | BH3 domain | ttcaaagcctggggcaagttcggaggcgccttttgccggtatgcg |
| | | PURB | gatgagatgaaagaa |
| 24 | DNA | NOXA | Atgcctgggaagaaggcgcgcaagaacgctcaaccgagccccgcg |
| | | | cgggctccagcagagctggaagtcgagtgtgctactcaactcagg |
| | | | agatttggagacaaactgaacttccggcagaaacttctgaatctg |
| | | | atatccaaactcttctgctcaggaacctga |
| 25 | DNA | HRK | Atgtgcccgtgccccctgcaccgcggccgcggccccccggccgtg |
| | | | tgcgcctgcagcgcgggtcgcctggggctgcgctcgtccgccgcg |
| | | | cagctcaccgccgcccggctcaaggcgctaggcgacgagctgcac |
| | | | cagcgcaccatgtggcggcgccgcgcgcggagccggagggcgccg |
| | | | gcgcccggcgcgctccccacctactggccttggctgtgcgcggcc |
| | | | gcgcaggtggcggcgctggcggcctggctgctcggcaggcggaac |
| | | | ttgtag |
| 26 | DNA | BAD | Atgttccagatcccagagtttgagccgagtgagcaggaagactcc |
| | | | agctctgcagagaggggcctgggccccagccccgcaggggacggg |
| | | | ccctcaggctccggcaagcatcatcgccaggccccaggcctcctg |
| | | | tgggacgccagtcaccagcaggagcagccaaccagcagcagccat |
| | | | catggaggcgctggggctgtggagatccggagtcgccacagctcc |
| | | | taccccgcggggacggaggacgacgaagggatgggggaggagccc |
| | | | agcccctttcggggccgttcgcgctcggcgccccccaacctctgg |
| | | | gcagcacagcgctatggccgcgagctccggaggatgagtgacgag |
| | | | tttgtggactcctttaagaagggacttcctcgcccgaagagcgcg |
| | | | ggcacagcaacgcagatgcggcaaagctccagctggacgcgagtc |
| | | | ttccagtcctggtgggatcggaacttgggcaggggaagctccgcc |
| | | | ccctcccagtga |
| 27 | DNA | BID | Atggactgtgaggtcaacaacggttccagcctcagggatgagtgc |
| | | | atcacaaacctactggtgtttggcttcctccaaagctgttctgac |
| | | | aacagcttccgcagagagctggacgcactgggccacgagctgcca |
| | | | gtgctggctccccagtgggagggctacgatgagctgcagactgat |
| | | | ggcaaccgcagcagccactcccgcttgggaagaatagaggcagat |
| | | | tctgaaagtcaagaagacatcatccggaatattgccaggcacctc |
| | | | gcccaggtcggggacagcatggaccgtagcatccctccgggcctg |
| | | | gtgaacggcctggccctgcagctcaggaacaccagccggtcggag |
| | | | gaggaccggaacagggacctggccactgccctggagcagctgctg |
| | | | caggcctaccctagagacatggagaaggagaagaccatgctggtg |
| | | | ctggccctgctgctggccaagaaggtggccagtcacacgccgtcc |
| | | | ttgctccgtgatgtctttcacacaacagtgaattttattaaccag |
| | | | aacctacgcacctacgtgaggagcttagccagaaatgggatggac |
| | | | tga |
| 28 | DNA | BIM | Atggcaaagcaaccttctgatgtaagttctgagtgtgaccgagaa |
| | | | ggtagacaattgcagcctgcggagaggcctccccagctcagacct |
| | | | ggggcccctacctccctacagacagagccacaaggtaatcctgaa |
| | | | ggcaatcacggaggtgaaggggacagctgcccccacggcagccct |
| | | | cagggcccgctggccccacctgccagccctggcccttttgctacc |
| | | | agatccccgcttttcatctttatgagaagatcctccctgctgtct |
| | | | cgatcctccagtgggtatttctcttttgacacagacaggagccca |
| | | | gcacccatgagttgtgacaaatcaacacaaaccccaagtcctcct |
| | | | tgccaggccttcaaccactatctcagtgcaatggcttccatgagg |
| | | | caggctgaacctgcagatatgcgcccagagatatggatcgcccaa |
| | | | gagttgcggcgtatcggagacgagtttaacgcttactatgcaagg |
| | | | agggtatttttgaataattaccaagcagccgaagaccacccacga |
| | | | atggttatcttacgactgttacgttacattgtccgcctggtgtgg |
| | | | agaatgcattga |
| 29 | DNA | BIK | Atgtctgaagtaagacccctctccagagacatcttgatggagacc |
| | | | ctcctgtatgagcagctcctggaacccccgaccatggaggttctt |
| | | | ggcatgactgactctgaagaggacctggaccctatggaggacttc |
| | | | gattctttggaatgcatggagggcagtgacgcattggccctgcgg |
| | | | ctggcctgcatcggggacgagatggacgtgagcctcagggccccg |
| | | | cgcctggcccagctctccgaggtggccatgcacagcctgggtctg |
| | | | gctttcatctacgaccagactgaggacatcagggatgttcttaga |
| | | | agtttcatggacggtttcaccacacttaaggagaacataatgagg |
| | | | ttctggagatccccgaaccccgggtcctgggtgtcctgcgaacag |
| | | | gtgctgctggcgctgctgctgctgctggcgctgctgctgccgctg |
| | | | ctcagcgggggcctgcacctgctgctcaagtga |
| 30 | DNA | PUMA | Atggcccgcgcacgccaggagggcagctccccggagcccgtagag |
| | | | ggcctggcccgcgacggcccgcgccccttcccgctcggccgcctg |
| | | | gtgccctcggcagtgtcctgcggcctctgcgagcccggcctggct |
| | | | gccgcccccgccgcccccaccctgctgcccgctgcctacctctgc |
| | | | gcccccaccgccccacccgccgtcaccgccgccctggggggttcc |
| | | | cgctggcctgggggtccccgcagccggccccgaggcccgcgcccg |
| | | | gacggtcctcagccctcgctctcgctggcggagcagcacctggag |
| | | | tcgcccgtgcccagcgccccgggggctctggcgggcggtcccacc |
| | | | caggcggccccgggagtccgcggggaggaggaacagtgggcccgg |
| | | | gagatcggggcccagctgcggcggatggcggacgacctcaacgca |
| | | | cagtacgagcggcggagacaagaggagcagcagcggcaccgcccc |
| | | | tcaccctggagggtcctgtacaatctcatcatgggactcctgccc |
| | | | ttacccaggggccacagagcccccgagatggagcccaattag |
| 31 | DNA | BMF | Atggagccatctcagtgtgtggaggagctggaggatgatgtgttc |
| | | | caaccagaggatggggagccggtgacccaacccgggagcttgctc |
| | | | tctgctgacctgtttgcccagagcctactggactgccccctcagc |
| | | | cgacttcagctcttccctctcacccactgctgtggccctggcctt |
| | | | cgacccaccagccaggaagacaaagctacccagactctcagccca |
| | | | gcctcccccagccaaggtgtcatgctgccttgtggggtgactgag |
| | | | gaaccccagcgactcttttatggcaatgctggctatcggcttcct |
| | | | ctccctgccagtttcccagcagtcttgcccattggggagcagccc |
| | | | cccgaagggcagtggcaacatcaagcagaggtacagattgcccga |
| | | | aagcttcagtgcattgcagaccagttccaccggcttcatgtgcag |
| | | | caacaccagcagaaccaaaatcgtgtgtggtggcagatcctcctc |
| | | | ttcctgcacaaccttgctttgaatggagaagagaacaggaacggg |
| | | | gcaggccctaggtga |
| 32 | DNA | Beclin-1 | atggaagggtctaagacgtccaacaacagcaccatgcaggtgagc |
| | | | ttcgtgtgccagcgctgcagccagcccctgaaactggacacgagt |
| | | | ttcaagatcctggaccgtgtcaccatccaggaactcacagctcca |
| | | | ttacttaccacagcccaggcgaaaccaggagagacccaggaggaa |
| | | | gagactaactcaggagaggagccatttattgaaactcctcgccag |
| | | | gatggtgtctctcgcagattcatccccccagccaggatgatgtcc |
| | | | acagaaagtgccaacagcttcactctgattggggaggcatctgat |
| | | | ggcggcaccatggagaacctcagccgaagactgaaggtcactggg |
| | | | gacctttttgacatcatgtcgggccagacagatgtggatcaccca |
| | | | ctctgtgaggaatgcacagatactcttttagaccagctggacact |
| | | | cagctcaacgtcactgaaaatgagtgtcagaactacaaacgctgt |
| | | | ttggagatcttagagcaaatgaatgaggatgacagtgaacagtta |
| | | | cagatggagctaaaggagctggcactagaggaggagaggctgatc |
| | | | caggagctggaagacgtggaaaagaaccgcaagatagtggcagaa |
| | | | aatctcgagaaggtccaggctgaggctgagagactggatcaggag |
| | | | gaagctcagtatcagagagaatacagtgaatttaaacgacagcag |
| | | | ctggagctggatgatgagctgaagagtgttgaaaaccagatgcgt |
| | | | tatgcccagacgcagctggataagctgaagaaaaccaacgtcttt |
| | | | aatgcaaccttccacatctggcacagtggacagtttggcacaatc |
| | | | aataacttcaggctgggtcgcctgcccagtgttcccgtggaatgg |
| | | | aatgagattaatgctgcttggggccagactgtgttgctgctccat |
| | | | gctctggccaataagatgggtctgaaatttcagagataccgactt |
| | | | gttccttacggaaaccattcatatctggagtctctgacagacaaa |
| | | | tctaaggagctgccgttatactgttctggggggttgcggtttttc |
| | | | tgggacaacaagtttgaccatgcaatggtggctttcctggactgt |
| | | | gtgcagcagttcaaagaagaggttgagaaaggcgagacacgtttt |
| | | | tgtcttccctacaggatggatgtggagaaaggcaagattgaagac |
| | | | acaggaggcagtggcggctcctattccatcaaaacccagtttaac |
| | | | tctgaggagcagtggacaaaagctctcaagttcatgctgacgaat |
| | | | cttaagtggggtcttgcttgggtgtcctcacaattttataacaaa |
| | | | tga |
| 33 | DNA | BNIP2 | Atggaaggtgtggaacttaaagaagaatggcaagatgaagatttt |
| | | | ccgatacctttaccagaagatgatagtattgaagcagatatacta |
| | | | gctataactggaccagaggaccagcctggctcactagaagttaat |
| | | | ggaaataaagtgagaaagaaactaatggctccagacattagcctg |
| | | | acactggatcctagtgatggctctgtattgtcagatgatttggat |
| | | | gaaagtggggagattgacttagatggcttagacacaccgtcagag |
| | | | aatagtaatgagtttgagtgggaagatgatcttccaaaacccaag |
| | | | actactgaagtaattaggaaaggctcaattactgaatacacagca |
| | | | gcagaggaaaaagaagatggacgacgctggcgtatgttcaggatt |
| | | | ggagaacaggaccacagggttgatatgaaggcaattgaaccctat |
| | | | aaaaaagttatcagccatgggggatattatggggatggattaaat |
| | | | gccattgttgtatttgctgtctgtttcatgcctgaaagtagtcag |
| | | | cctaactatagatacctgatggacaatctttttaaatatgttatt |
| | | | ggcactttggagctattagtagcagaaaactacatgatagtttat |
| | | | ttaaatggtgcaacaactcgaagaaaaatgcccagtctgggatgg |
| | | | ctcaggaaatgttatcagcaaattgatagaaggttacggaaaaat |
| | | | ctaaaatccctaatcattgtacatccttcttggtttatcagaaca |
| | | | cttctggctgttacaagaccatttattagctcgaaattcagccaa |
| | | | aaaattagatacgtgtttaatttggcagaactagcagaacttgtc |
| | | | cccatggaatacgttggcataccagaatgcataaaacaagttgat |
| | | | caagaacttaatggaaaacaagatgaaccgaaaaatgaacagtaa |
| 34 | DNA | BNIP3L | Atgtcgtcccacctagtcgagccgccgccgcccctgcacaacaac |
| | | | aacaacaactgcgaggaaaatgagcagtctctgcccccgccggcc |
| | | | ggcctcaacagttcctgggtggagctacccatgaacagcagcaat |
| | | | ggcaatgataatggcaatgggaaaaatggggggctggaacacgta |
| | | | ccatcctcatcctccatccacaatggagacatggagaagattctt |
| | | | ttggatgcacaacatgaatcaggacagagtagttccagaggcagt |
| | | | tctcactgtgacagcccttcgccacaagaagatgggcagatcatg |
| | | | tttgatgtggaaatgcacaccagcagggaccatagctctcagtca |
| | | | gaagaagaagttgtagaaggagagaaggaagtcgaggctttgaag |
| | | | aaaagtgcggactgggtatcagactggtccagtagacccgaaaac |
| | | | attccacccaaggagttccacttcagacaccctaaacgttctgtg |
| | | | tctttaagcatgaggaaaagtggagccatgaagaaagggggtatt |
| | | | ttctccgcagaatttctgaaggtgttcattccatctctcttcctt |
| | | | tctcatgttttggctttggggctaggcatctatattggaaagcga |
| | | | ctgagcacaccctctgccagcacctactga |
| 35 | DNA | NOXA upstream | atgcctggcaagaaggcccggaagaacgcccaaccttctccagct |
| | | domain | agagcccctgcc |
| | | 57 nucleotide | |
| 36 | DNA | NOXA | atgcctggcaagaaggcccggaagaacgcccaaccttctccagcc |
| | | upstream domain | agagcc |
| | | 51 nucleotide | |
| 37 | DNA | NOXA | Cagaagctgctgaacctgatcagcaagctgttctgcagcggcacc |
| | | downstream | |
| | | domain | |
| | | 45 nucleotide | |
| 38 | DNA | NOXA | Aacctgatcagcaagctgttctgcagcggcacc |
| | | downstream | |
| | | domain | |
| | | 33 nucleotide | |
| 39 | DNA | NOXA (NOXA) | Atgcctggcaagaaggcccggaagaacgcccaaccttctccagct |
| | | | agagcccctgccgaactggaagtggaatgtgccacacagctgcgg |
| | | | agattcggcgacaagctgaacttccggcagaagctgctgaacctg |
| | | | atcagcaagctgttctgcagcggcacc |
| 40 | DNA | NOXA (HRK) | atgcctggcaagaaggcccggaagaacgcccaaccttctccagct |
| | | | agagcccctgcctcgtccgccgcgcagctcaccgccgcccggctc |
| | | | aaggcgctaggcgacgagctgcaccagcagaagctgctgaacctg |
| | | | atcagcaagctgttctgcagcggcacc |
| 41 | DNA | NOXA (BAD) | atgcctggcaagaaggcccggaagaacgcccaaccttctccagct |
| | | | agagcccctgccctctgggcagcacagcgctatggccgcgagctc |
| | | | cggaggatgagtgacgagtttgtggaccagaagctgctgaacctg |
| | | | atcagcaagctgttctgcagcggcacc |
| 42 | DNA | NOXA (BID) | Atgcctggcaagaaggcccggaagaacgcccaaccttctccagct |
| | | | agagcccctgccgacatcatccggaatattgccaggcacctcgcc |
| | | | caggtcggggacagcatggaccgtagccagaagctgctgaacctg |
| | | | atcagcaagctgttctgcagcggcacc |
| 43 | DNA | NOXA (BIM) | Atgcctggcaagaaggcccggaagaacgcccaaccttctccagcc |
| | | | agagccgacatgaggcccgagatctggattgcccaagagctgaga |
| | | | agaatcggcgacgagttcaacgcctactacgccagacggaacctg |
| | | | atcagcaagctgttctgcagcggcacc |
| 44 | DNA | NOXA (PURB) | atgcctggcaagaaggcccggaagaacgcccaaccttctccagct |
| | | | agagcccctgccttcaaagcctggggcaagttcggaggcgccttt |
| | | | tgccggtatgcggatgagatgaaagaacagaagctgctgaacctg |
| | | | atcagcaagctgttctgcagcggcacc |
| 45 | Amino | NOXA (NOXA) | MPGKKARKNAQPSPARAPAELEVECATQLRRFGDKLNFRQKLLNL |
| | acid | | ISKLFCSGT |
| 46 | Amino | NOXA (HRK) | MPGKKARKNAQPSPARAPAMSSAAQLTAARLKALGDELHQQKLLN |
| | acid | | LISKLFCSGT |
| 47 | Amino | NOXA (BAD) | MPGKKARKNAQPSPARAPALWAAQRYGRELRRMSDEFVDQKLLNL |
| | acid | | ISKLFCSGT |
| 48 | Amino | NOXA (BID) | MPGKKARKNAQPSPARAPADIIRNIARHLAQVGDSMDRSQKLLNL |
| | acid | | ISKLFCSGT |
| 49 | Amino | NOXA (BIM) | MPGKKARKNAQPSPARADMRPEIWIAQELRRIGDEFNAYYARRNL |
| | acid | | ISKLFCSGT |
| 50 | Amino | NOXA (PURB) | MPGKKARKNAQPSPARAPAFKAWGKFGGAFCRYADEMKEQKLLNL |
| | acid | | ISKLFCSGT |
| 51 | Amino | Granzyme B | MQPILLLLAFLLLPRADAGEIIGGHEAKPHSRPYMAYLMIWDQKS |
| | acid | | LKRCGGFLIRDDFVLTAAHCWGSSINVTLGAHNIKEQEPTQQFIP |
| | | | VKRPIPHPAYNPKNFSNDIMLLQLERKAKRTRAVQPLRLPSNKAQ |
| | | | VKPGQTCSVAGWGQTAPLGKHSHTLQEVKMTVQEDRKCESDLRHY |
| | | | YDSTIELCVGDPEIKKTSFKGDSGGPLVCNKVAQGIVSYGRNNGM |
| | | | PPRACTKVSSFVHWIKKTMKRY |
| 52 | Amino | Inactive BH-3- | MPGKKARKNAQPSPARAPAELEVECATQERREGDKENFRQKLLNL |
| | acid | only protein | ISKLFCSGT |
| 53 | Amino | Inactive BH-3- | QERREGDKEN |
| | acid | only protein | |
| 54 | DNA | Inactive BH-3- | agagcggagagagggcgacaaagaga |
| | | only protein | |
| 55 | DNA | Inactive BH-3- | Atgcctggcaagaaggcccggaagaacgcccaaccttctccagct |
| | | only protein | agagcccctgccgaactggaagtggaatgcgccacacaagagcgg |
| | | | agagagggcgacaaagagaacttccggcagaagctgctgaacctg |
| | | | atcagcaagctgttctgcagcggcacc |
| 56 | DNA | Tbax without | Atggacgggtccggggagcagcccagaggcggggggcccaccagc |
| | | transmembrane | tctgagcagatcatgaagacaggggcccttttgcttcagggtttc |
| | | domain sequence | atccaggatcgagcagggcgaatggggggggaggcacccgagctg |
| | | | gccctggacccggtgcctcaggatgcgtccaccaagaagctgagc |
| | | | gagtgtctcaagcgcatcggggacgaactggacagtaacatggag |
| | | | ctgcagaggatgattgccgccgtggacacagactccccccgagag |
| | | | gtctttttccgagtggcagctgacatgttttctgacggcaacttc |
| | | | aactggggccgggttgtcgcccttttctactttgccagcaaactg |
| | | | gtgctcaaggccctgtgcaccaaggtgccggaactgatcagaacc |
| | | | atcatgggctggacattggacttcctccgggagcggctgttgggc |
| | | | tggatccaagaccagggtggttgggacggcctcctctcctacttt |
| | | | gggacgcccacgtggcagacc |

In case there is a discrepancy between the sequence list disclosed herein and the sequence listing provided as part of the Sequence Listing Compliance Standard, the sequence listing provided as part of the Sequence Listing Compliance Standard is decisive. Alternatively, the sequence list disclosed herein and above may be used. Moreover, the molecule names in the sequence list disclosed herein (e.g., “Granzyme A,” “Granzyme A leader peptide” and so forth) have been included for the convenience of the reader, without any (functional) relationship to the sequence provided.

### BRIEF DESCRIPTION OF THE DRAWINGS

FIG. 1. Expression of Serpin B9 across lymphoma, multiple myeloma and solid cancer cell lines. Western blot staining showing Serpin B9 is expressed in whole cell lysates of lymphoma and some multiple myeloma (top two panels), and several solid cancer cell lines (bottom two panels).

FIG. 2. Levels of Serpin B9 expression per human cancer type. By combining immunoblot and gene expression data, a cut-off (log 2 RPKM 0) could be set to distinguish cell lines that express Serpin B9 from cell lines lacking gene and protein expression. Based on this cut-off, the fraction of Serpin B9-expressing cell lines within each cancer type included in the Cancer Cell Line Encyclopedia (CCLE) dataset was determined. Abbreviations: DLBCL, diffuse large B cell lymphoma; AML, acute myeloid leukemia; B-ALL, B cell acute lymphocytic leukemia; CML, chronic myeloid leukemia; DLBCL, diffuse large B cell lymphoma; NSC, non-small cell (lung cancer); T-ALL, T cell acute lymphocytic leukemia.

FIG. 3. Overexpression of Serpin B9 impairs sensitivity to granzyme B-induced cell death. (Panel A) Serpin B9 expression in untreated (WT), mock-transduced, and Serpin B9 overexpressing (Pmscv-Serpin B9) malignant melanoma Mewo cells, as determined by immunoblotting. (Panel B) Specific apoptosis induced by streptolysin O (SLO)-mediated introduction of 200 Nm recombinant granzyme B into WT and Serpin B9-overexpressing Mewo cells, normalized to the conditions with SLO only. (Panel C) Specific apoptosis in WT and Serpin B9-overexpressing Mewo cells after 24 hours of co-culture with the NK cell line YT-Indy at the indicated effector:target (E:T) ratios. Data in Panels B and C are average values of three to four independent experiments (+S.E.M.), compared with an unpaired student's t-test (Panel B) or two-way ANOVA with Sidak multiple testing correction (Panel C).

FIG. 4. Granzyme B-independent killing by NK cells may be death receptor-mediated and depends on the target cells. (Panel A) Specific apoptosis of WT (left) or Serpin B9-overexpressing (right) cells after 24 hours of co-culture with WT YT-Indy NK cells or YT-Indy NK cells in which granzyme B was knocked out (GrBKO), at the indicated effector:target (E:T) ratios. (Panel B) As in Panel A, with B-cell non-Hodgkin lymphoma OCI-Ly7 cells as target cells. Data in Panels A and B are average values of two to four independent experiments (+S.E.M.), compared with a two-way ANOVA with Sidak multiple testing correction.

FIG. 5. Knockdown of Serpin B9 expression in lymphoma increases killing by chimeric antigen receptor (CAR) T cells. (Panel A) Representative staining of Serpin B9 expression in the B-cell non-Hodgkin lymphoma cell line OCI-Ly7 after siRNA-mediated knockdown of Serpin B9, compared to untreated cells and cells electroporated with non-targeting siRNA. (Panel B) Specific apoptosis of OCI-Ly7 cells electroporated with non-targeting or Serpin B9 siRNA, after 4 hours co-culture with anti-CD20 CAR expressing T cells from three healthy donors at the indicated effector:target (E:T) ratios.

FIG. 6. Serpin B9 expression does not affect the intrinsic apoptosis machinery. Specific apoptosis of WT malignant melanoma Mewo cells (solid lines) and Mewo cells overexpressing Serpin B9 (Mewo-B9, dotted lines) after 24 hours treatment with the indicated concentrations of MCL-1 inhibitor S63845, BCL-XL inhibitor A-1155463, alone or combined.

FIG. 7. Exogeneous NOXA sequesters the pro-survival MCL-1. Western blots after immunoprecipitation (IP) experiments for myeloid cell leukemia 1 (MCL-1), or an isotype control (IgG), in multiple myeloma L363 cells treated with 15 ng/ml streptolysin O (SLO) and either or not with 8 Mm of synthetic NOXA (having the same sequence as wildtype NOXA). Shown are the indicated proteins that were pulled down (IP).

FIG. 8. Exogenous NOXA induces apoptosis in cancer cells. Multiple myeloma (L363 and MM1.S, top panel) and diffuse large B cell lymphoma cell lines (Ly7 and Ly10, bottom panel) were treated with varying concentrations of synthetic NOXA (having the same sequence as wildtype NOXA) in the presence or absence of sub-lethal concentrations of streptolysin O (SLO). Sub lethal concentrations of SLO were tested per cell line and was determined to result in less than 10% specific apoptosis due to treatment with SLO only. Specific apoptosis was calculated by measuring the altered percentage of viable cells compared with untreated cells. Values are averages of three to eight experiments per cell line with SEM.

FIG. 9. Strategy to redirect pro-apoptotic NOXA variants to cytotoxic granules in T and NK cells. Different variations of NOXA constructs were made, wherein the NOXA gene is placed behind the granzyme B sequence, followed by an HA-tag for easy visualization by microscopy, flow cytometry and Western blot. The ribosomal skipping sequence T2A allows generation of two different proteins from one Mrna (e.g., in this case generating a separate GFP protein). Abbreviations: GFP, green fluorescent protein; HA, human influenza hemagglutinin.

FIG. 10. Binding of NOXA variations having replaced BH3 effector domains for different pro-survival/anti-apoptotic BCL-2 family proteins. Here, the BH3 domain of BH3-only molecule NOXA is replaced with BH3 effector domains that determine binding to, hence inactivation of, the six different pro-survival BCL-2 family proteins (BCL-2, BCL-XL, BCL-W, MCL-1, BFL-1 and BCL-B). − indicates no binding, +++ indicates strong binding and + indicates intermediate binding. BH3 effector domains with a 20 amino acid length are used from BH3-only proteins NOXA, HRK, BAD and BID. For BIM a 25 amino acid length was used. Purine-rich element binding protein B (PURB) is a BH3 effector domain that has specificity for only BCL-2. Abbreviations: BCL-2, B cell lymphoma; BCL-XL, B-cell lymphoma extra-large; HRK, hara-kiri; MCL-1, myeloid cell leukemia 1; BFL-1, BCL-2 related gene in fetal liver; BAD, BCL-2 associated agonist of cell death; BID, BH3-interacting domain death agonist, BIM, BCL-2-like protein 11; WT, wildtype.

FIG. 11. Introduced NOXA localizes to cytotoxic granules in T cells. (Panel A) CD8 T cells transduced with the NOXA construct show correlated expression of green fluorescent protein (GFP) and human influenza hemagglutinin (HA). (Panel B) ImageStream analysis reveals that NOXA is localized to lysosomal-associated membrane protein 1 (LAMP-1)-positive cytotoxic granules in CD8 T cells transduced with the NOXA construct.

FIG. 12. Introduced NOXA in NK cells promotes cell death in target cells. YT-Indy NK cells transduced with the NOXA(WT) construct kill H929 multiple myeloma and OCI-Ly7 diffuse large B cell lymphoma (DLBCL) cells in a dose-dependent manner.

FIG. 13. Use of the granzyme-perforin trafficking system localizes the cargo into the granules. B-cell maturation antigen (BCMA) CAR T cells are transduced with the Mscarlet TRACKER construct. Confocal microscopy shows that granzyme B delivers Mscarlet into LAMP1-positive granules, while GFP is localized to the cytosol.

FIG. 14. Multiple myeloma (MM) cells take up the cargo delivered using the granzyme-perforin trafficking system and survive the interaction. Representative of three experiments is shown. BCMA CAR T cells transduced with the Mscarlet TRACKER construct were co-cultured with MM cell line NCI-H929 for 4 or 16 hours and analyzed by flow cytometry. As control, untransduced BCMA CAR T cells were used. Viable BCMA CAR T cells (CD3+) were plotted together with viable NCI-H929 cells (CD3−). The gate indicates NCI-H929 cells that have taken up Mscarlet upon interaction with BCMA CAR T cells and survived the interaction. Representative of three experiments is shown.

FIG. 15. BCMA CAR T cells transduced with targeting NOXA constructs induce specific apoptosis in multiple myeloma cells. NOXA constructs were developed targeting MCL-1 (NOXA WT, “NOXA”), all pro-survival proteins [NOXA(BIM), “Snoxa” ] or nothing as control [NOXA(3E), “Inoxa” ] (Panel A). Co-culture of BCMA CAR T cells transduced with the NOXA constructs and primary MM cells leads to specific apoptosis after 24 hours of co-culture with four different patient samples plotted separately (Panel B) or averaged (Panel C).

FIG. 16. BCMA CART cells transduced with targeting NOXA constructs induce specific apoptosis in multiple myeloma cells sensitive to MCL-1 inhibition. NOXA constructs were developed targeting MCL-1 (NOXA WT, “NOXA”), all pro-survival proteins [NOXA(BIM), “Snoxa” ] or nothing as control [NOXA(3E), “Inoxa” ]. Co-culture of BCMA CAR T cells transduced with NOXA or Snoxa constructs and H929 (Panel A) or INA6 (Panel B) cells leads to specific apoptosis after 24 hours of co-culture. Data are average of 2-3 experiments.

FIG. 17. BCMA CAR T cells transduced with NOXA(BIM) constructs induce specific apoptosis in multiple myeloma cells insensitive to MCL-1 inhibition. NOXA constructs were developed targeting MCL-1 (NOXA WT, “NOXA”), all pro-survival proteins [NOXA(BIM), “Snoxa” ] or nothing as control [NOXA(3E), “Inoxa” ]. Co-culture of BCMA CAR T cells transduced with NOXA or Snoxa constructs and MM1S (Panel A) or RPMI8226-LUC (Panel B) cells leads to specific apoptosis after 24 hours of co-culture. Data are average of 2-3 experiments.

FIG. 18. Tumor growth in mice following treatment with BCMA CART cells. Mice with developed tumors were treated with NOXA constructs were developed targeting MCL-1 (NOXA WT, “NOXA”), or inactive NOXA was used as control [NOXA(3E), “Inoxa” ] by i.v. injection. Bioluminescence signal was measured at different time points as a sign of tumor growth.

### CLAUSES

Herein, clauses are embodiments of the disclosure. Features of clauses (embodiments) herein can be combined.

Clause 1. Nucleic acid molecule comprising:
1) a first nucleotide sequence encoding a granule-localizing domain;
2) a second nucleotide sequence encoding a pro-apoptotic protein comprising a B-cell lymphoma 2 (BCL-2) homology 3 (BH3) effector domain.

Clause 2. Nucleic acid molecule according to clause 1, wherein the BH3 effector domain binds a multidomain BCL-2 family protein and/or a BH3 domain-binding groove of a multidomain BCL-2 family protein, preferably a pro-survival BCL-2 family protein and/or a BH3 domain-binding groove of a pro-survival BCL-2 family protein.

Clause 3. Nucleic acid molecule according to any one of the previous clauses, wherein the granule-localizing domain is a granzyme protein or a leader peptide thereof, and/or wherein the first nucleotide sequence is:
a nucleotide sequence encoding granzyme A, preferably having at least 90% sequence identity with SEQ ID NO:1, or a nucleotide sequence encoding a leader peptide of granzyme A, preferably having at least 90% sequence identity with SEQ ID NO:2;
a nucleotide sequence encoding granzyme B, preferably having at least 90% sequence identity with SEQ ID NO:3, or a nucleotide sequence encoding a leader peptide of granzyme B, preferably having at least 90% sequence identity with SEQ ID NO:4;
a nucleotide sequence encoding granzyme H, preferably having at least 90% sequence identity with SEQ ID NO:5, or a nucleotide sequence encoding a leader peptide of granzyme H, preferably having at least 90% sequence identity with SEQ ID NO:6;
a nucleotide sequence encoding granzyme K, preferably having at least 90% sequence identity with SEQ ID NO:7, or a nucleotide sequence encoding a leader peptide of granzyme K, preferably having at least 90% sequence identity with SEQ ID NO:8;
a nucleotide sequence encoding granzyme M, preferably having at least 90% sequence identity with SEQ ID NO:9, or a nucleotide sequence encoding a leader peptide of granzyme M, preferably having at least 90% sequence identity with SEQ ID NO:10;
a nucleotide sequence encoding granulysin, preferably having at least 90% sequence identity with SEQ ID NO:11, or a nucleotide sequence encoding leader peptide of granulysin, preferably having at least 90% sequence identity with SEQ ID NO:12;
a nucleotide sequence encoding serglycin, preferably having at least 90% sequence identity with SEQ ID NO:13, or a nucleotide sequence encoding a leader peptide of serglycin, preferably having at least 90% sequence identity with SEQ ID NO:14;
a nucleotide sequence encoding perforin, preferably having at least 90% sequence identity with SEQ ID NO:15, or a nucleotide sequence encoding a leader peptide of perforin, preferably having at least 90% sequence identity with SEQ ID NO:16; and/or
a nucleotide sequence encoding for an N-Acetylglucosamine-1 (GlcNAc-1) phosphotransferase binding domain, preferably having at least 90% sequence identity with SEQ ID NO:17.

Clause 4. Nucleic acid molecule according to any one of the previous clauses, wherein the BH3 effector domain is a BH3 effector domain of a BH3-only pro-apoptotic protein and/or a BH3 effector domain of a purine rich element binding protein (PUR), preferably PURbeta (PURB), wherein the BH3-only pro-apoptotic protein is preferably chosen from BCL-2-like protein 11 (BIM), BH3-interacting domain death agonist (BID), BCL-2 interacting killer (BIK), p53 upregulated modulator of apoptosis (PUMA), BCL-2 associated agonist of cell death (BAD), phorbol-12-myristate-13-acetate-induced protein 1 (NOXA), BCL-2-modifying factor (BMF), harakiri (HRK), Beclin-1, BCL-2/adenovirus E1B 19 kDa protein-interacting protein (BNIP)2, BNIP3, and/or BNIP3L, and/or wherein the BH3 effector domain is encoded by a nucleotide sequence having at least 90% sequence identity with:
SEQ ID NO:18;
SEQ ID NO:19;
SEQ ID NO:20;
SEQ ID NO:21;
SEQ ID NO:22; and/or
SEQ ID NO:23.

Clause 5. Nucleic acid molecule according to any one of the previous clauses,
wherein the second nucleotide sequence encodes for a BH3-only pro-apoptotic protein wherein the BH3 effector domain is substituted by a BH3 effector domain of another BH3-only pro-apoptotic protein, wherein the BH3-only pro-apoptotic protein is preferably chosen from:
   NOXA, and/or a BH3-only protein encoded by a nucleotide sequence having at least 90% sequence identity with SEQ ID NO:24;
   HRK, and/or a BH3-only protein encoded by a nucleotide sequence having at least 90% sequence identity with SEQ ID NO:25;
   BAD, and/or a BH3-only protein encoded by a nucleotide sequence having at least 90% sequence identity with SEQ ID NO:26;
   BID, and/or a BH3-only protein encoded by a nucleotide sequence having at least 90% sequence identity with SEQ ID NO:27;
   BIM, and/or a BH3-only protein encoded by a nucleotide sequence having at least 90% sequence identity with SEQ ID NO:28;
   BIK, and/or a BH3-only protein encoded by a nucleotide sequence having at least 90% sequence identity with SEQ ID NO:29;
   PUMA, and/or a BH3-only protein encoded by a nucleotide sequence having at least 90% sequence identity with SEQ ID NO:30;
   BMF, and/or a BH3-only protein encoded by a nucleotide sequence having at least 90% sequence identity with SEQ ID NO:31;
   Beclin-1, and/or a BH3-only protein encoded by a nucleotide sequence having at least 90% sequence identity with SEQ ID NO:32;
   BNIP2, and/or a BH3-only protein encoded by a nucleotide sequence having at least 90% sequence identity with SEQ ID NO:33;
   BNIP3;
   BNIP3L, and/or a BH3-only protein encoded by a nucleotide sequence having at least 90% sequence identity with SEQ ID NO:34,
wherein preferably the BH3 effector domain is substituted by:
   a BH3 effector domain of NOXA, preferably encoded by a nucleotide sequence having at least 90% sequence identity with SEQ ID NO:18;
   a BH3 effector domain of HRK, preferably encoded by a nucleotide sequence having at least 90% sequence identity with SEQ ID NO:19;
   a BH3 effector domain of BAD, preferably encoded by a nucleotide sequence having at least 90% sequence identity with SEQ ID NO:20;
   a BH3 effector domain of BID, preferably encoded by a nucleotide sequence having at least 90% sequence identity with SEQ ID NO:21;
   a BH3 effector domain of BIM, preferably encoded by a nucleotide sequence having at least 90% sequence identity with SEQ ID NO:22; or
   a BH3 effector domain of PURB, preferably encoded by a nucleotide sequence having at least 90% sequence identity with SEQ ID NO:23.

Clause 6. Nucleic acid molecule encoding a pro-apoptotic protein, wherein the pro-apoptotic protein is encoded by a nucleotide sequence comprising:
1) a BH3 effector domain encoding sequence;
2) an upstream domain encoding sequence that is located upstream of the BH3 effector domain encoding sequence;
3) a downstream domain encoding sequence that is located downstream of the BH3 effector domain encoding sequence,
wherein the upstream domain encoding sequence encodes:
   an upstream domain of NOXA, preferably having at least 90% sequence identity with SEQ ID NO:35 and/or SEQ ID NO:36,
and/or wherein the downstream domain encoding sequence encodes
   a downstream domain of NOXA, preferably having at least 90% sequence identity with SEQ ID:37 and/or SEQ ID:38;
wherein the BH3 effector domain encoding sequence encodes:
   a BH3 effector domain of HRK, preferably having at least 90% sequence identity with SEQ ID NO:19;
   a BH3 effector domain of BAD, preferably having at least 90% sequence identity with SEQ ID NO:20;
   a BH3 effector domain of BID, preferably having at least 90% sequence identity with SEQ ID NO:21;
   a BH3 effector domain of BIM, preferably having at least 90% sequence identity with SEQ ID NO:22; and/or
   a BH3 effector domain of PURB, preferably having at least 90% sequence identity with SEQ ID NO:23.

Clause 7. Nucleic acid molecule according to clause 6, wherein the pro-apoptotic protein is encoded by a nucleotide sequence comprising:
an upstream domain encoding sequence of NOXA, preferably having at least 90% sequence identity with SEQ ID NO:35, a BH3 effector domain encoding sequence of HRK, preferably having at least 90% sequence identity with SEQ ID NO:19, and a downstream domain encoding sequence of NOXA, preferably having at least 90% sequence identity with SEQ ID NO:37, preferably the nucleotide sequence has at least 90% sequence identity with SEQ ID NO:40;
an upstream domain encoding sequence of NOXA, preferably having at least 90% sequence identity with SEQ ID NO:35, a BH3 effector domain encoding sequence of BAD, preferably having at least 90% sequence identity with SEQ ID NO:20, and a downstream domain encoding sequence of NOXA, preferably having at least 90% sequence identity with SEQ ID NO:37, preferably the nucleotide sequence has at least 90% sequence identity with SEQ ID NO:41;
an upstream domain encoding sequence of NOXA, preferably having at least 90% sequence identity with SEQ ID NO:35, a BH3 effector domain encoding sequence of BID, preferably having at least 90% sequence identity with SEQ ID NO:21, and a downstream domain encoding sequence of NOXA, preferably having at least 90% sequence identity with SEQ ID NO:37, preferably the nucleotide sequence has at least 90% sequence identity with SEQ ID NO:42;
an upstream domain encoding sequence of NOXA, preferably having at least 90% sequence identity with SEQ ID NO:36, a BH3 effector domain encoding sequence of BIM, preferably having at least 90% sequence identity with SEQ ID NO:22, and a downstream domain encoding sequence of NOXA, preferably having at least 90% sequence identity with SEQ ID NO:38, preferably the nucleotide sequence has at least 90% sequence identity with SEQ ID NO:43; or
an upstream domain encoding sequence of NOXA, preferably having at least 90% sequence identity with SEQ ID NO:35, a BH3 effector domain encoding sequence of PURB, preferably having at least 90% sequence identity with SEQ ID NO:23, and a downstream domain encoding sequence of NOXA, preferably having at least 90% sequence identity with SEQ ID NO:37, preferably the nucleotide sequence has at least 90% sequence identity with SEQ ID NO:44.

Clause 8. Nucleic acid molecule according to clause 6 or 7, comprising a further nucleotide sequence that encodes a granule-localizing domain,
wherein the granule-localizing domain is preferably a granule-localizing domain as defined by clause 3, and/or
wherein the further nucleotide sequence is preferably a first nucleotide sequence as defined by clause 3.

Clause 9. Nucleic acid molecule according to any one of the clauses 1-5 and 8, further comprising one or more of:
1) a nucleotide sequence encoding a linker molecule, wherein the linker molecule is preferably located between the granule-localizing domain and the pro-apoptotic protein, wherein the linker molecule is preferably one or more of
   ubiquitin;
   a peptide consisting of the amino acid Glycine, Asparagine and/or Serine;
   a peptide consisting of the amino acid Threonine and/or Alanine, and/or Glycine; and
   a peptide consisting of Glycine-Serine residues (GS linker), preferably a GS linker with a length of 1-53 amino acids;
2) a nucleotide sequence encoding a cleavage site for an enzyme, wherein the cleavage site is preferably located between the granule-localizing domain and the pro-apoptotic protein, wherein the cleavage site is preferably one or more of a cleavage site for a protease, most preferably a cleavage site for a protease selected from the group consisting of a caspase, a cathepsin, an enterokinase, furin, factor Xa, a matrix metalloproteinase, and an aggrecanase, preferably a caspase, most preferably caspase 3;
3) a nucleotide sequence encoding a promoter operatively linked to a nucleotide sequence encoding the granule-localizing domain and/or a nucleotide sequence encoding the pro-apoptotic protein.

Clause 10. Pro-apoptotic protein comprising a BH3 effector domain, wherein the pro-apoptotic protein is encoded by a nucleic acid molecule according to clause 6 or 7, preferably having an amino acid sequence with at least 90% sequence identity with any one of SEQ ID NO:45, 46, 47, 48, 49, −50.

Clause 11. Pro-apoptotic construct, preferably a fusion protein, comprising:
1) a granule-localizing domain, and
2) a pro-apoptotic protein comprising a BH3 effector domain,
wherein the granule-localizing domain is preferably:
   a granule-localizing domain as defined by clause 3; and/or
   a granule-localizing domain encoded by a first nucleotide sequence as defined by clause 3,
and/or wherein the pro-apoptotic protein is preferably:
   a pro-apoptotic protein comprising a BH3 effector domain as defined by clause 2 and/or 4;
   a BH3-only pro-apoptotic protein as defined by clause 5;
   a pro-apoptotic protein encoded by the nucleic acid molecule as defined by clauses 6 and/or 7; and/or
   a pro-apoptotic protein according to clause 10,
wherein the pro-apoptotic construct preferably further comprises:
   a linker molecule between the granule-localizing domain and the pro-apoptotic protein, preferably a linker molecule as defined by clause 9; and/or
   a cleavage site between the granule-localizing domain and the pro-apoptotic protein, preferably a cleavage site as defined by clause 9.

Clause 12. Nucleic acid delivery construct comprising the nucleic acid molecule according to any one of clauses 1-9, wherein the nucleic acid delivery construct is preferably chosen from one or more of a plasmid, a recombinant adenovirus, an adeno-associated virus (AAV), a retrovirus, a lentivirus, a herpes simplex virus, and a vaccinia virus, preferably a lentivirus.

Clause 13. Immune cell, wherein the immune cell:
comprises the nucleic acid molecule according to any one of clauses 1-9;
expresses a pro-apoptotic protein encoded by the nucleic acid molecule as defined by any of clauses 1-9;
expresses a pro-apoptotic protein according to clause 10;
expresses a pro-apoptotic construct according to clause 11;
comprises the nucleic acid delivery construct according to clause 12;
has an immune receptor, preferably an engineered receptor, most preferably a chimeric antigen receptor; and/or
induces apoptosis of cancer cells, wherein the apoptosis is preferably mediated by inactivating one or more pro-survival BCL-2 family proteins, preferably one or more pro-survival BCL-2 family proteins, most preferably one or more of BCL-2, C-cell lymphoma-extra large (BCL-XL), BCL-W, myeloid cell leukemia 1 (MCL-1), BCL-2 related gene in fetal liver (BFL-1), and BCL-B.

Clause 14. Immune cell according to clause 13, wherein the immune cell is a human T cell or human NK cell, and/or wherein the immune cell is for use in medical therapy, preferably cancer therapy, more preferably cancer immunotherapy, wherein the cancer preferably is one or more of melanoma, liver cancer, breast cancer, colorectal cancer, lung cancer, prostate cancer, multiple myeloma, lymphoma, or leukemia.

Clause 15. Nucleic acid molecule according to any one of clauses 1-9, pro-apoptotic protein as defined by any one of clauses 1-9, pro-apoptotic protein according to clause 10, pro-apoptotic construct according to clause 11, nucleic acid delivery construct according to clause 12, or immune cell according to clause 13 or 14, for use in medical therapy, preferably for use in cancer therapy, more preferably for use in cancer immunotherapy, wherein the cancer preferably is one or more of melanoma, liver cancer, breast cancer, colorectal cancer, lung cancer, prostate cancer, multiple myeloma, lymphoma or leukemia.

### EXPERIMENTAL EXAMPLES

### Experimental Example 1

### 1. Background

In this experimental example, the role of the natural granzyme inhibitor Serpin B9 in the resistance of cancer cells to chimeric antigen receptor (CAR) T cells and T cell receptor (TCR) engineered T cells was investigated. Moreover, ways to bypass possible resistance caused by Serpin B9 was also investigated.

### 2. Methods

### 2.1. Cell Culture and Chemicals

Cell lines were cultured in Dulbecco's Modified Eagle Medium (DMEM, Life Technologies), Iscove's Modified Dulbecco's Medium (IMDM, life Technologies), or RPMI 1640 GlutaMAX HEPES culture medium (Life Technologies), supplemented with 10-20% fetal bovine serum (FBS, Sigma) and 100 μg/ml penicillin-streptomycin (p/s, Gibco/Life Technologies). Human healthy donor peripheral blood mononuclear cells (PBMCs) were isolated from buffy coats (Sanquin, Amsterdam, the Netherlands) using Ficoll-Paque according to the manufacturer's protocol. PBMCs were cultured in RPMI with 2.5% pooled AB+ human serum (IPLA-CSER, Innovative Research), 50 Mm β-mercaptoethanol (Life Technologies) and 1% p/s.

### 2.2. Immunoblotting

For Western blot analysis, cells were lysed in buffer containing 1% Nonidet P-40 and proteins were separated using SDS-PAGE (Mini-PROTEAN® TGX™ Precast Gels, Bio-Rad), transferred to low florescence PVDF membranes (Bio-Rad), blocked in PBS containing 2% non-fat dry milk, and stained using the following antibodies: mouse anti-Serpin B9 (Invitrogen), mouse anti-α-tubulin (Cell Signaling), and goat anti-mouse-680RD (LI-COR Biosciences). Infrared imaging was used for detection (Odyssey Sa, LI-COR Biosciences). Analysis and quantification were performed using LI-COR Image Studio software.

### 2.3. Generation of CD20 CAR T Cells

The CD20 CAR construct (pBu-CD20-CAR) was generated by cloning single chain variable fragments from anti-CD20 antibody Rituximab into a pBullet vector containing a D8α-41BB-CD3-ζ signaling cassette. Phoenix-Ampho packaging cells were transfected with gag-pol (pHit60), env (P-COLT-GALV) and pBu-CD20-CAR, using FugeneHD transfection reagent (Promega). Human PBMCs were pre-activated with 30 ng/ml anti-CD3 (OKT3, Miltenyi) and 50 IU/ml IL-2 (Sigma) and subsequently transduced two times with viral supernatant in the presence of 6 μg/ml polybrene (Sigma) and 50 U/ml IL-2. Transduced T cells were expanded using 50 U/ml IL-2 and anti CD3/CD28 dynabeads (Thermo Fisher), and CD20-CAR-expressing cells were selected by treatment with 80 μg/ml neomycin. T cells were further expanded using rapid expansion protocol as described elsewhere.

### 2.4. Overexpression and Knockdown of Serpin B9

Generation of the retroviral vector Pmscv-Serpin B9 is described elsewhere. Virus production was performed as described for the CD20-CAR construct. Subsequently, Mewo cells were transduced two times with viral supernatant in the presence of 6 μg/ml polybrene, and stably overexpressing cells were selected using 1 μg/ml puromycin (Sigma). In order to knock down Serpin B9 expression, the ON-TARGETplus Human SERPIN B9 siRNA SMARTpool (L-015400-00-0005, Dharmacon) was electroporated into OCI-Ly7 cells using a Neon transfection system 10 μl kit (Thermo Fischer Scientific), at 1150 V, with 2×30 ms pulses.

### 2.5. Co-Culture Experiments

The effect of Serpin B9 overexpression in Mewo cells on killing by cytotoxic cells was determined by co-culturing wildtype (WT) and Serpin B9-overexpressing Mewo with the YT-Indy NK cell line. Similarly, the effect of Serpin B9 knockdown in OCI-Ly7 cells was investigated by co-culture with YT-Indy cells or CD20 CAR T cells. Effector and target cells were combined in ratios 1:1, 3:1, and 6:1, and co-culture took place for 4 hours (lymphoma cell lines) or 24 hours (Mewo).

### 2.6. Apoptosis Staining and Flow Cytometry

Assessment of cell viability took place by staining with 15 Nm DiOC6 (Thermo Scientific) and 20 Nm TO-PRO-3 (Thermo Scientific), followed by flow cytometric analysis (BD FACSCanto II or BD LSRFortessa, BD Biosciences). Specific apoptosis was calculated by determining the altered percentage of DiOC6+/TO-PRO-3⁻ (live) cells compared to untreated cells, using the formula (% cell death in treated cells−% cell death in control)/% viable cells control*100. In co-culture experiments, target cells were identified by flow cytometric surface staining with CD19-BV421 (Sony Biotechnology) (lymphoma cell lines), or by staining with Cell Trace Violet (Invitrogen) (Mewo) prior to adding effector cells. CD20 CAR T cells were characterized by staining with CD4-Pacific Blue (Biolegend), CD8-PE/Cy7 (BD), and biotinylated protein L (Genscript) with streptavidin-PE (Thermo Fisher). For intracellular staining of Serpin B9, cells were fixed and permeabilized using BD Cytofix/Cytoperm (BD Biosciences) and stained with mouse anti-Serpin B9 (Invitrogen), mouse anti-IgG Isotype (Southern Biotech). Flow cytometry data analysis took place using FlowJo.

2.7. Introduction of Exogenous Granzyme B into Target Cells

In order to determine the effect of Serpin B9 overexpression or knockdown on granzyme-mediated killing, the pore forming Streptolysin O (Sigma) was used to facilitate entry of exogenous Granzyme B (Enzo) into target cells. SLO was activated with 10 Mm DTT for 20 minutes at RT, and subsequently diluted in serum-free DMEM to a final concentration of 4 U/ml. Target cells were incubated with SLO and 200 Nm Granzyme B for 30 minutes at 37° C., after which FBS-containing medium was added to inactivate SLO. After 24 hours, apoptosis staining was performed to measure target cell viability using flow cytometry.

### 2.8. Statistical Analysis

Statistical analysis was performed using GraphPad Prism version 8.3. Unpaired groups were compared with a Student's t-test. For comparison of more than two groups, a two-way ANOVA was used.

### 3. Results

### 3.1. Serpin B9 is Expressed Across Lymphoma and in Some Multiple Myeloma and Solid Cancer Cell Lines

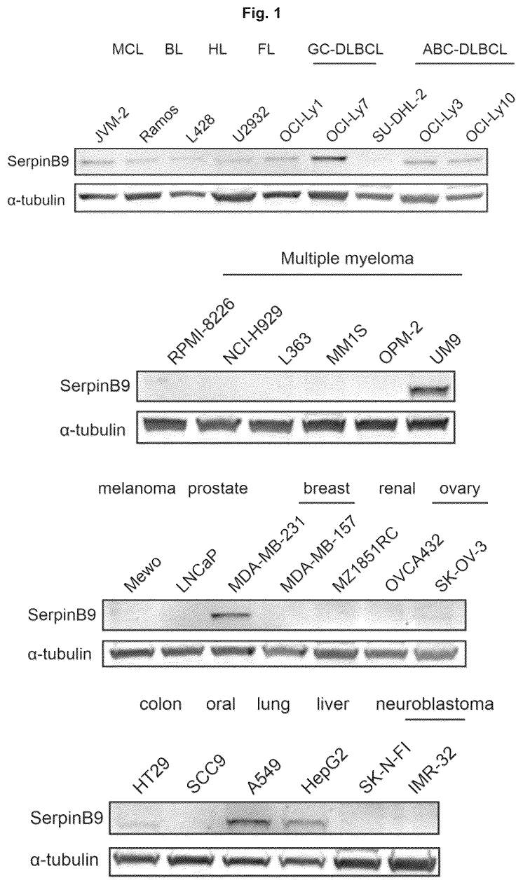

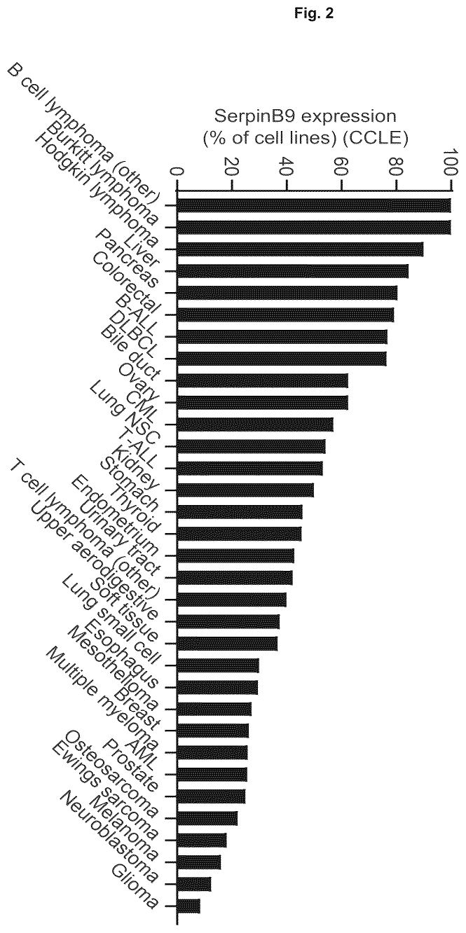

Serpin B9 is normally expressed in cytotoxic lymphocytes (CL), NK cells, antigen-presenting cells, endo- and mesothelial sites, and immune privileged sites. In order to determine the frequency of Serpin B9 expression in malignant cells of different tumor types, Serpin B9 expression was measured in panels of lymphoma, multiple myeloma and solid cancer cell lines (FIG. 1). In lymphoma, Serpin B9 was ubiquitously expressed, with Serpin B9 levels varying from almost undetectable (SU-DHL-2) to very high (OCI-Ly7). In multiple myeloma, one (UM9) out of six tested cell lines expressed Serpin B9. In a panel of solid cancer cell lines, clear Serpin B9 expression was observed in breast cancer cell line MDA-MB-231, lung cancer cell line A549, and liver carcinoma cell line HepG2, and lower levels were found in SK-OV-3 (ovary carcinoma) and HT29 (colon carcinoma). Together, these data indicate that Serpin B9 is expressed across lymphomas, and in some multiple myeloma and solid cancers. In addition, by combining immunoblot and gene expression data, a cutoff (log₂ RPKM 0) could be set to distinguish cell lines that express SERPIN B9 from cell lines lacking gene and protein expression. Based on this cutoff, the fraction of Serpin B9-expressing cell lines within each cancer type included in the CCLE dataset was determined (FIG. 2). This analysis predicts expression of Serpin B9 in 75-100% of B cell lymphoma and leukemia (Burkitt lymphoma, Hodgkin lymphoma, B-ALL, DLBCL, “B cell lymphoma other”) cell lines. Additionally, >75% Serpin B9 expression was observed in liver, pancreas, and colorectal cancer. In other cancer types, the fraction of Serpin B9 expression was lower, with large variation of expression within cancer types (FIG. 2).

### 3.2. Overexpression of Serpin B9 Impairs Sensitivity to Granzyme B-Induced Cell Death

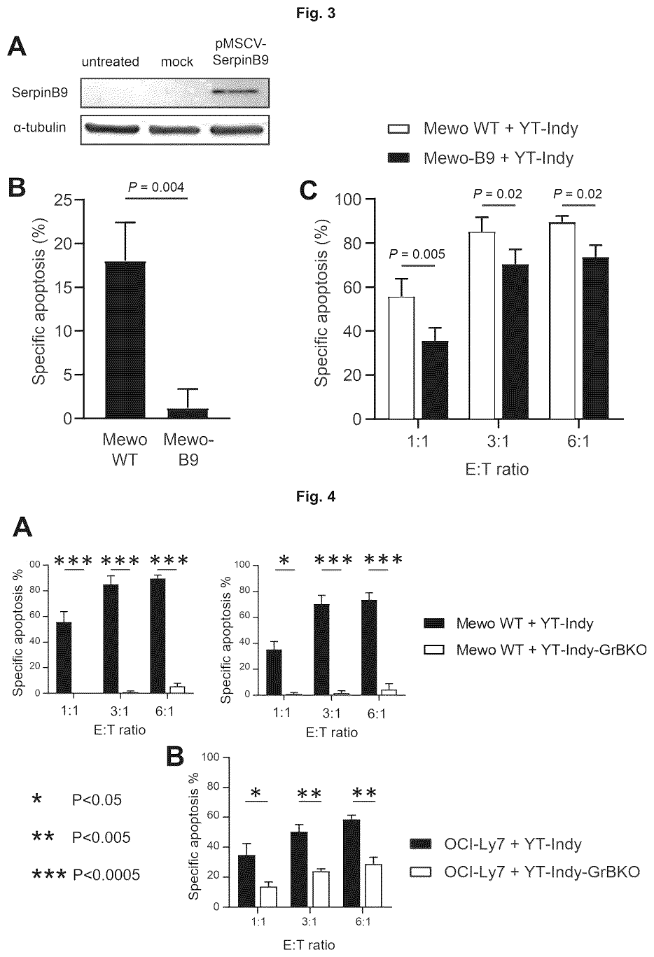

In order to examine the role of Serpin B9 in Granzyme B-mediated killing, Serpin B9 was stably overexpressed in Mewo, a melanoma cell line lacking endogenous Serpin B9 expression (FIG. 3, Panel A). Sensitivity of the WT and Serpin B9-overexpressing Mewo cell lines to Granzyme B was first assessed by introducing recombinant Granzyme B using streptolysin O (SLO), a bacterial pore-forming exotoxin. In this experiment, Serpin B9 overexpression rendered the cell line completely resistant to Granzyme B-mediated killing (FIG. 3, Panel B). Next, the cell lines were co-cultured with NK cell line YT-Indy, and significantly reduced killing was observed in Serpin B9-overexpressing cells (FIG. 3, Panel C). Co-culture of Mewo with a Granzyme B-knockout YT-Indy cell line (YT-Indy-Granzyme BKO) completely abrogated cell death (FIG. 4, Panel A), indicating that the observed target cell killing was fully dependent on Granzyme B. In contrast to Mewo, DLBCL cell line OCI-Ly7 has high endogenous expression of Serpin B9 (FIG. 1). Specific apoptosis observed upon co-culture of OCI-Ly7 was not fully abrogated when using YT-Indy-Granzyme BKO (FIG. 4, Panel B), suggesting that other cell death pathways are still effectively engaged in OCI-Ly7 in the absence of Granzyme B. Besides Granzyme B, NK cells kill by expressing death receptor ligands, such as Fas ligand, which may induce extrinsic apoptosis in target cells.

### 3.3. Knockdown of Serpin B9 Expression in Lymphoma Cells Increases Killing by CAR T Cells

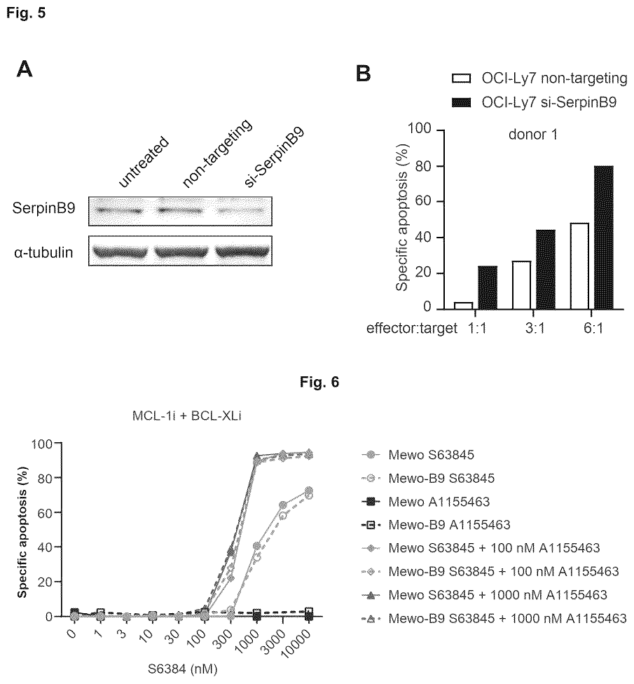

To study the role of Serpin B9 in CAR T cell therapy resistance, we performed siRNA-mediated knockdown of Serpin B9 in OCI-Ly7 (FIG. 5, Panel A). In addition, we stably expressed an anti-CD20 CAR in T cells from healthy donors. The used CAR contains the scFv sequence from anti-CD20 antibody rituximab, as well as a 4-1BB co-stimulatory domain. When OCI-Ly7 cells expressing normal or reduced levels of Serpin B9 were cultured in the presence of CD20-CAR T cells from healthy donors, the OCI-Ly7 cells underwent apoptosis in a concentration-dependent manner (FIG. 5, Panel B). Notably, knockdown of Serpin B9 increased CD20-CAR T cell-mediated killing of OCI-Ly7 cells (FIG. 5, Panel B). These results indicate that Serpin B9 expression, which is abundant in lymphomas, may impair killing by CAR T cells.

3.4. Serpin B9 Overexpression does not Impair the Intrinsic Apoptosis Pathway

It has been shown that Serpin B9 expression, which is widespread across cancers, may confer resistance of cancer cells against Granzyme B-mediated killing by gene-engineered T cells as well as NK cells. In addition to Granzyme B, apoptosis can be induced in tumor cells through activation of the extrinsic and intrinsic apoptosis pathways. Although Bid cleavage by Granzyme B can indirectly lead to intrinsic apoptosis, activation of both extrinsic and intrinsic apoptosis pathways may not depend on Granzyme B functionality. Treatment of Mewo cells with MCL-1 (563845) and BCL-XL (A-1155463) inhibitors reveals high sensitivity to combined MCL-1 and BCL-XL inhibitor treatment (FIG. 6). No difference between WT and Serpin B9-overexpressing Mewo with regard to BCL-2 family inhibitor sensitivity was observed. These results suggest that Granzyme B-resistance resulting from Serpin B9 overexpression may be circumvented by activating the intrinsic apoptosis pathways directly.

### 4. Summary

Expression of Serpin B9 in a panel of human cancer cell lines was demonstrated by Western blot and revealed expression in a range of cancer types, such as in most lymphoma cell lines, certain multiple myeloma, and in a selection of solid cancer cell lines. Using modified cell lines wherein Serpin B9 was either inhibited or overexpressed, it was shown that Serpin B9-overexpressing cancer cells may be less sensitive to exogenously delivered Granzyme B or to Granzyme B-mediated killing by NK cells and NY-ESO-1 engineered T cells, as compared to melanoma cells lacking Serpin B9. Conversely, knockdown of Serpin B9 in diffuse large B cell lymphoma (DLBCL) rendered these cells more sensitive toward killing by anti-CD20 CAR T cells, as compared to DLBCL with high expression of Serpin B9. These results indicate that Serpin B9 expression, which is abundant in different cancer types, may impair killing by engineered cytotoxic lymphocytes including CAR T cells. Finally, it was found that, regardless of Serpin B9 expression, cancer cells may remain equally sensitive to inhibitors of pro-survival BCL-2 family proteins, such as BCL-2, MCL-1 and BCL-XL, suggesting that this cell death pathways may be engaged to kill cancer cells that are resistant to engineered cytotoxic lymphocytes.

BH3-only proteins and/or modified variants thereof are established herein as more effective in killing cancer cells, in particular, in the presence of inhibitors of apoptosis such as Serpin B9. Among the possible explanation of these surprising findings, the following has been considered:
BH3-only proteins do not require cleavage in the cytosol to initiate apoptosis, e.g., as opposed to caspases;
BH3-only proteins do not induce toxicity in the effector cell or neighboring cells, as BH3-only proteins do not have a direct degrading or pore-forming effect. In comparison, caspases may degrade various molecules also outside the cancer cell. Moreover, BCL-2 pore formers (i.e., BAX, BAK) may insert into membranes and mitochondria outside of the cancer cell;
CAR T cells have shorter contacts with target cells as compared to conventional T cells, therefore the granzyme-perforin pathway (leading to activation of the intrinsic apoptosis pathway) plays a more important role. BH3-only proteins are therefore more effective than other pro-apoptotic molecules, e.g., such as those involved in the death receptor pathway, for cancer immunotherapy, preferably CAR T cell therapy or CAR NK cell therapy.

### Experimental Example 2

### 1. Background

Based on the findings herein disclosed, Serpin B9 is a likely mediator of resistance toward cytotoxic lymphocytes such as engineered (CAR) T cells or NK cells, providing a novel rationale to improve the killing capacity of engineered cytotoxic lymphocytes by circumventing Serpin B9-mediated inhibition of pro-apoptotic molecules including Granzyme B. This method makes use of the granzyme-perforin trafficking system to deliver pro-apoptotic molecules into cancer cells, e.g., after a cytotoxic lymphocyte engages with a cancer cell. Subsequently, the pro-apoptotic molecules may inactivate pro-survival BCL-2 family proteins in the cancer cell and hereby promote the intrinsic apoptosis pathway of killing. In the process, the pro-apoptotic molecules may overcome the effect of natural inhibitors such as Serpin B9. Additionally, the use of variants of BCL-2 family proteins having a replaced BH3 effector domain with far potent pro-apoptotic and killing capacity is disclosed herein.

### 2. Results

### 2.1. Exogenous NOXA Enters Tumor Cells Through Perforin-Like Pores, Sequesters MCL-1 and Induces Apoptosis of the Tumor Cell

Importantly, it was found that the inactivation of pro-survival BCL-2 family proteins may lead to efficient apoptosis in target cells, irrespective of the presence of natural inhibitors such as Serpin B9. As proof for this, it was first assessed whether a BH3-only BCL-2 family protein, for example, NOXA, can kill tumor cells when it enters cells through membrane pores. To visualize entry of NOXA into target cells, synthetic NOXA (having same sequence as wildtype NOXA) labelled with the fluorophore TAMRA (NOXA-TMR) was used. Since perforin is highly unstable, the bacterial pore-forming protein streptolysin O (SLO) was used, which creates pores in cell membranes of roughly equal size as perforin pores and allows passive diffusion of proteins into the cell. NOXA-TMR is significantly smaller (around 10 kDa) than Granzyme B (around 32 kDa) so it should be no problem for NOXA to enter cells through perforin or SLO-pores. Multiple myeloma (MM; L363) or diffuse large B cell lymphoma (DLBCL; Ly10) cell lines were incubated with NOXA-TMR and either or not a sub-lethal concentration of SLO. Using confocal fluorescence microscopy, it was confirmed that NOXA-TMR was efficiently localized to the cytoplasm in the presence of SLO, while without SLO it remained at the cell surface. Further proof that NOXA entered tumor cells through SLO-pores was obtained from immunoprecipitation (IP) experiments. Here, L363 cells were treated with SLO and either or not synthetic NOXA. An IP for MCL-1, the main natural binding partner of NOXA, using the lysate of treated cells clearly showed the interaction of exogenously delivered NOXA with MCL-1 in the cytoplasm (FIG. 7).

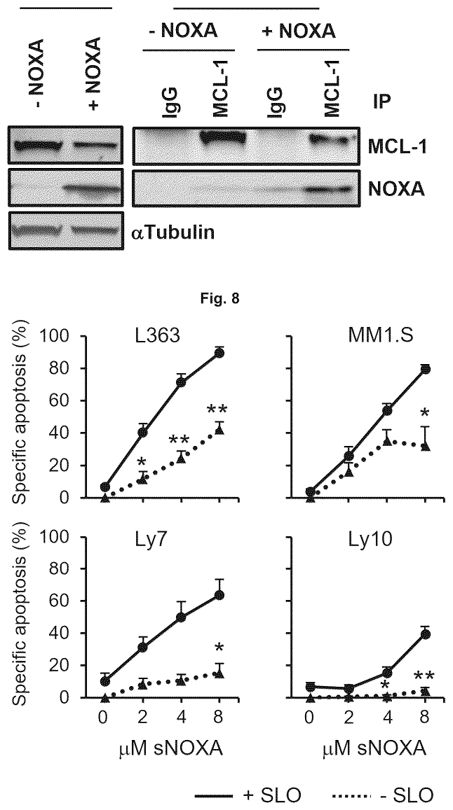

After confirming that a BH3-only BCL-2 family protein, for example, synthetic NOXA, enters cells through pores in the cell membrane, it was examined whether it could also induce apoptosis. Multiple myeloma MM and diffuse large B cell lymphoma (DLBCL) cell lines were treated with varying concentrations of synthetic NOXA in the presence or absence of sub-lethal concentrations of SLO. These sub-lethal concentrations of SLO were tested per cell line and was determined to result in less than 10% specific apoptosis due to treatment with SLO only. This revealed that exogenous NOXA induces apoptosis, although the sensitivity to NOXA differed per cell line (FIG. 8). Interestingly, the sensitivity to NOXA was highly comparable to sensitivity toward the novel MCL-1-specific inhibitor A1210477. Combined, the IP experiments (FIG. 7) and experiments with NOXA and MCL-1 inhibitor (FIG. 8), demonstrate that the mechanism of action regarding tumor cell killing by exogenous NOXA is through inhibition of pro-survival MCL-1. Finally, comparisons were made as to tumor cell killing by NOXA with cell killing induced by Granzyme B. MM or DLBCL cells were treated with SLO in combination with synthetic NOXA, recombinant Granzyme B or a combination of both. As expected, NOXA and Granzyme B both induced apoptosis of the tumor cells. The addition of Granzyme B did not hamper NOXA activity. Surprisingly, the combined treatment showed increased tumor cell death compared to the single treatments, suggesting that NOXA and Granzyme B act through different apoptotic pathways that act in concert.

### 2.2. NOXA Construct Localizes to Cytotoxic Granules in T or NK Cells and Promotes Killing of Cancer Cells

Next, it was found that pro-apoptotic proteins may be introduced into target cells by preceding their domains with specific granule-localizing domains, such as granule-localizing domains, which are essential for pro-apoptotic molecules to localize to cytotoxic granules. For this purpose, constructs were generated wherein the BH3-only BCL-2 family protein, for example, NOXA, was placed behind Granzyme B for localization into cytotoxic granules of cytotoxic lymphocytes. The Granzyme B sequence allows localization of NOXA into Granzyme B-positive cytotoxic granules and will then be cleaved off after arrival in these granules.

At the same time, it was found that the pro-apoptotic activity of BH3-only proteins, including NOXA, may be enhanced by replacing their BH3 effector domain. This follows the premise that the BH3 effector domain (e.g., of BH3-only proteins) largely determines the specificity and affinity for multidomain effector proteins of the BCL-2 family, including the for pro-survival BCL-2 family proteins. Normally, NOXA promotes cell killing by neutralizing pro-survival BCL-2 family protein MCL-1. However, NOXA does not inhibit other BCL-2 family proteins (e.g., BCL-2, BCL-B, BCL-W, BCL-XL, and BFL-1), even though many tumor types also depend on expression of these other BCL-2 family proteins for survival. Therefore, in addition to the wild type (WT) NOXA, denoted as NOXA(NOXA), a number of NOXA variants were generated, characterized in that the variants have a different ability to bind to and inactivate anti-apoptotic/pro-survival BCL-2 family proteins:
NOXA(PURB): a variant comprising a BH3 effector domain from the transcriptional activator protein Pur-beta that is predicted to have specificity for only BCL-2, hence, has a different pro-apoptotic activity than NOXA(NOXA).
NOXA(HRK), a variant with the BH3 effector domain from HRK that binds BCL-XL, hence, also has a different pro-apoptotic activity than NOXA(NOXA).
NOXA(BAD), a variant with the BH3 effector domain from BAD that binds BCL-2, BCL-XL, and BCL-W, hence, has moderate/high pro-apoptotic activity.
NOXA(BID), a variant with the BH3 effector domain from BID that binds BFL-1, BCL-W, BCL-XL, BCL-2, and MCL-1, hence, has high pro-apoptotic activity.
NOXA(BIM): a variant with the BH3 effector domain from BIM that binds all pro-survival BCL-2 family proteins, namely MCL-1, MFL-1, BCL-B, BCL-2, BCL-XL, and BCL-W, hence, has very high pro-apoptotic activity.

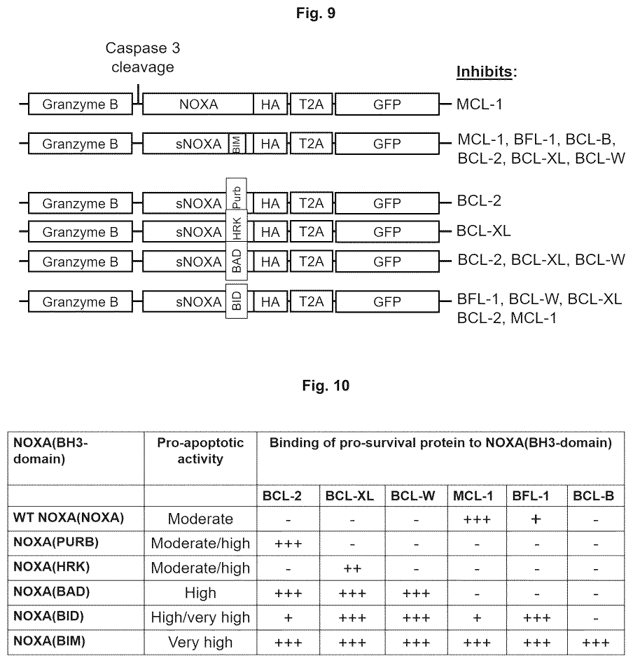

In the different variations of the NOXA constructs, the NOXA gene is placed behind the Granzyme B sequence, followed by an HA-tag for easy visualization by microscopy, flow cytometry and Western blot. The ribosomal skipping sequence T2A allows generation of two different proteins from one Mrna. As a result, GFP is not directed to granules by Granzyme B, but expressed in the cytosol as a separate protein. A cleavage site between Granzyme B and NOXA, in this case a caspase 3 cleavage site, allows cleavage of both proteins upon arrival in the target cell. Since active caspase 3 is only expressed in target cells undergoing apoptosis, premature cleavage in the transduced cytotoxic cells will not occur (FIG. 9). The pro-apoptotic activity and the associated binding of the different NOXA variants for pro-survival proteins of the BCL-2 family is summarized in FIG. 10.

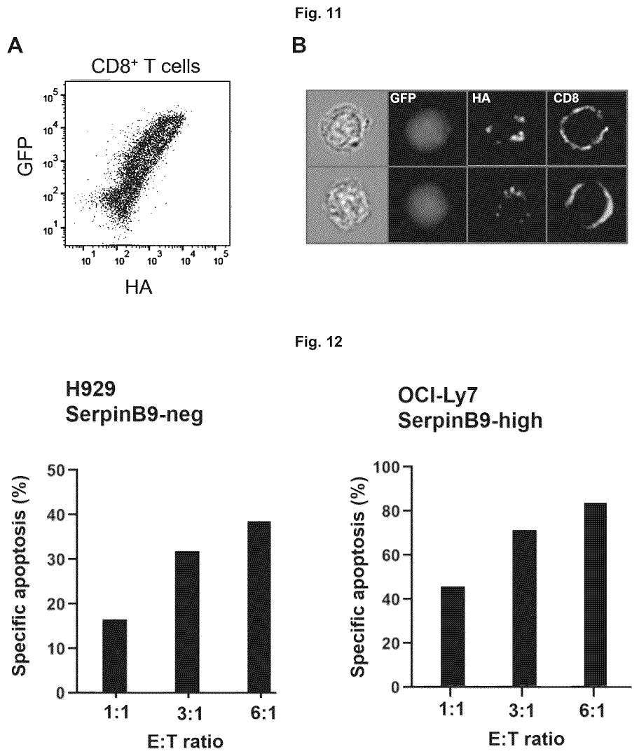

To show the potency of the novel constructs, primary CD8 T cells and the NK cell line YT-Indy with the wildtype NOXA constructs were lentivirally transduced, and revealed that NOXA indeed localizes to lysosomal-associated membrane protein 1 (LAMP-1)-positive cytotoxic granules in CD8 T cells and in NK cells (CD8 T cells shown in FIG. 11). Importantly, it was found thatNK cells transduced with the novel NOXA construct killed H929 multiple myeloma (Serpin B9-neg) and OCI-Ly7 (Serpin B9-high) cells (FIG. 12). These data demonstrate that it is possible to direct pro-apoptotic NOXA into cancer cells though interaction with engineered NK or T cells and subsequently boost their killing. Since the variants of NOXA (e.g., NOXA(BIM), NOXA(BID), NOXA(BAD) have a replaced BH3 effector domain with improved binding to BCL-2 family proteins these variants are more efficient in inducing apoptosis than NOXA and larger therapeutic effects on cancer cell killing can be expected in patients when using these variants.

### 2.3. NOXA(BIM) Constructs Localize to Cytotoxic Granules in T Cells and Promotes Specific Apoptosis of Multiple Myeloma Cells

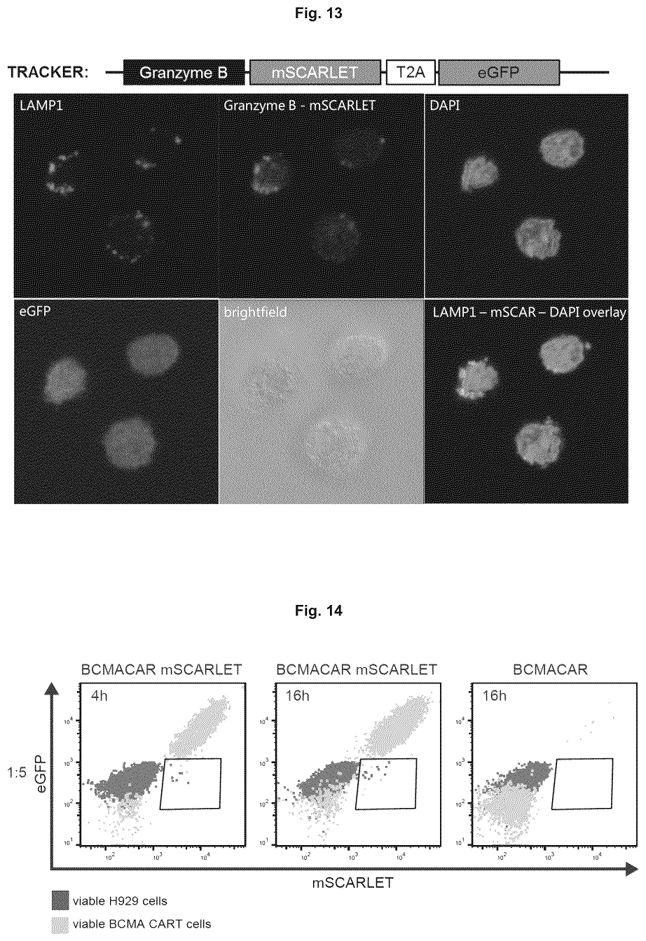

To show that NOXA(BIM) is localized to cytotoxic granules in CAR T cells and transferred to cancer cells after contact, NOXA was replaced with the fluorescent Mscarlet molecule in a lentiviral construct. Confocal microscopy showed that granzyme B delivers Mscarlet into LAMP1-positive granules in B-cell maturation antigen (BCMA) CAR T cells transduced with the Mscarlet TRACKER construct, while GFP is localized to the cytosol (FIG. 13).

BCMA CAR T cells transduced with the Mscarlet TRACKER construct were co-cultured with multiple myeloma (MM) cell line NCI-H929 for 4 or 16 hours and analyzed by flow cytometry. As control, untransduced BCMA CAR T cells were used. Viable BCMA CAR T cells (CD3+) were plotted together with viable NCI-H929 cells (CD3−). The gate indicates NCI-H929 cells that have taken up Mscarlet upon interaction with BCMA CAR T cells and survived the interaction (FIG. 14). A similar experiment was performed, but in the presence of caspase-inhibitor QVD to prevent apoptosis of the targeted MM cells. The accumulation of SCARLET-positive MM cells was found in time upon co-culture with BCMA CAR T cells.

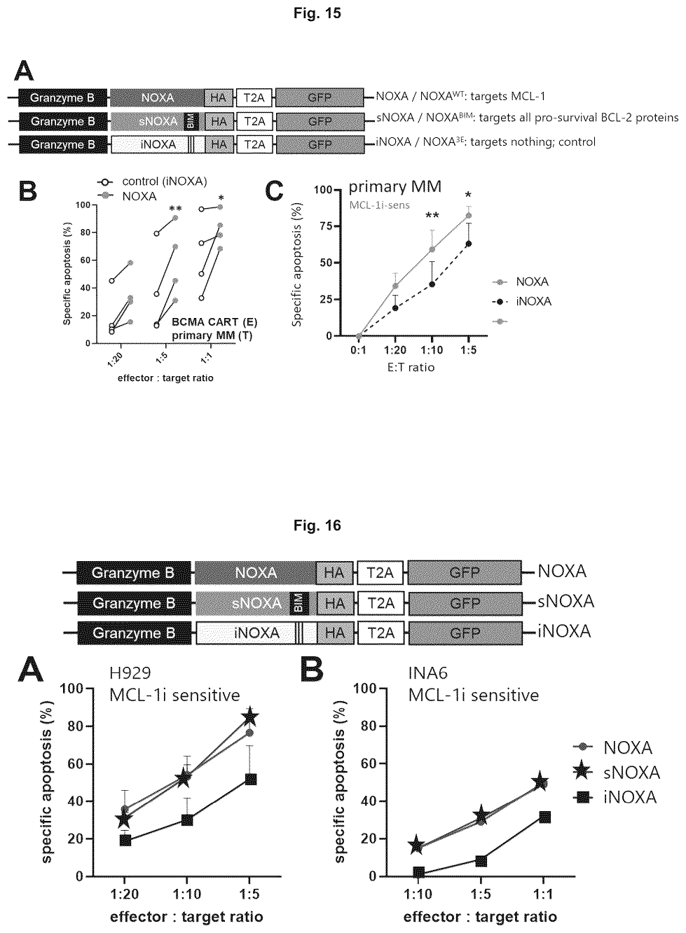

Different variations of NOXA constructs were made, wherein the NOXA gene is placed behind the granzyme B sequence, followed by an HA-tag for easy visualization. The ribosomal skipping sequence T2A allows generation of two different proteins from one Mrna (e.g., in this case generating a separate GFP protein). Constructs were developed targeting MCL-1 (NOXA WT, “NOXA”), all pro-survival proteins [NOXA(BIM), “Snoxa” ] or nothing as control [NOXA(3E), “Inoxa” ] (FIG. 15, Panel A). It was found that NOXA (detected by HA-staining) localizes to (LAMP-1-positive) cytotoxic granules in BCMA CAR T cells transduced with the granzyme B-NOXA lentiviral construct. Furthermore, it was found that co-culture of BCMA CAR T cells transduced with the NOXA constructs and primary MM cells leads to specific apoptosis after 24 hours of co-culture with four different patient samples plotted separately (FIG. 15, Panel B) or averaged (FIG. 15, Panel C).

It appeared that the use of a granzyme-localizing domain for the delivery of (modified) NOXA is far more effective and causes no or less effects, as compared to other delivery systems such as transmembrane-localizing domains.

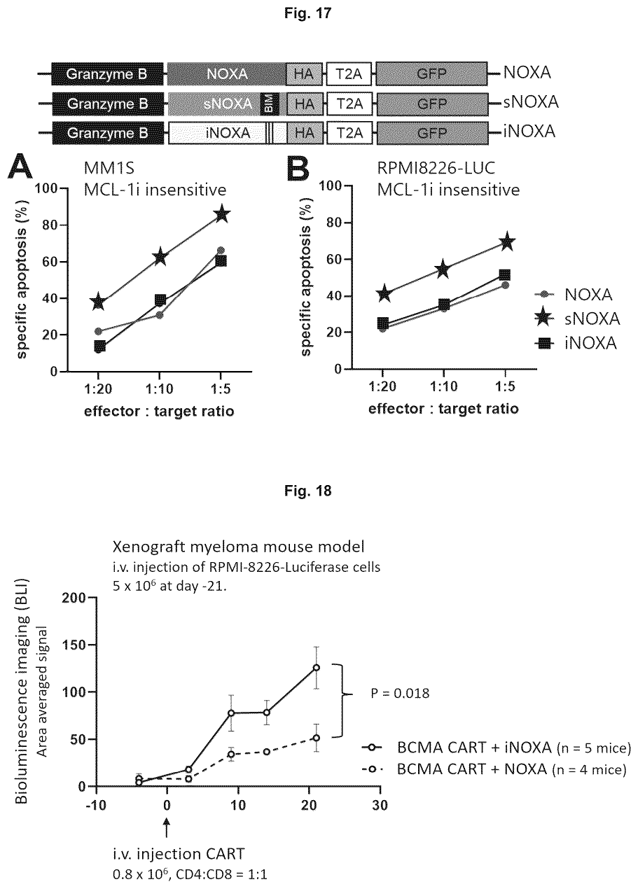

Next, BCMA CAR T cells transduced with the aforementioned NOXA, Snoxa or Inoxa constructs were cocultured with the H929 or the INA6 MM cell lines that are sensitive to MCL-1 inhibition (MCL-li) (FIG. 16). Based on the specific apoptosis after 24 hours of co-culture, it was found that specific apoptosis is induced by NOXA and Snoxa in both H929 (FIG. 16, Panel A) or INA6 (FIG. 16, Panel B) cells. A similar experiment was performed using the MM1 S and RPMI8226-LUC MM cell lines, which are insensitive to MCL-1 inhibition (FIG. 17). It was found that MM1 S (FIG. 17, Panel A) and RPMI8226-LUC (FIG. 17, Panel B) cells insensitive to MCL-1 inhibition by NOXA, are sensitive to inhibition via Snoxa (i.e., NOXA(BIM)).

### 3. Summary

Exogenous pro-apoptotic proteins, particularly those that inactivate pro-survival BCL-2 family proteins (e.g., NOXA and related), was shown to efficiently induce apoptosis in cancer cells, irrespective of Serpin B9 inhibitory activity. When preceded by a granule localizing domain (such as comprised in Granzyme B), the pro-apoptotic proteins readily localize into cytotoxic granules in T cells and NK cells and bind to the pro-survival BCL-2 family proteins to induce apoptosis. Finally, it was shown that modified variants of pro-apoptotic proteins, i.e., having replaced BH3 effector domains, have an altered killing capacity due to the modified specificity and affinity for pro-survival BCL-2 family proteins. In particular variants of BH3-only proteins, including but not limited to NOXA, are likely most efficient in cancer killing. The current data hold proof that this novel strategy can be applied to empower engineered cytotoxic lymphocytes (e.g., engineered T or NK cells), in cancer killing irrespective of natural inhibitors such as Serpin B9. Foremost, NOXA variants comprising a BIM effector domain appear most effective and were shown to induce specific apoptosis in cancer cells insensitive to MCL-1. This indicates that all tumor types could be sensitive to this modified version of NOXA having the BIM BH3 effector domain.

### Experimental Example 3

### 1. Background

The current example compares the efficiency of different constructs based on several BCL-2 family proteins and their BH3 effector domains (i.e., BIM, PUMA, BID, BAX, NOXA), or diphtheria toxin A, placed behind Granzyme B in translocating and inducing apoptosis in cancer cells.

BIM is a BH3-only protein for which the binding specificity appears to be defined entirely by its effector BH3 domain. The BH3 effector domains of BIM binds to all of its pro-survival relatives and also weakly to BAX. Similarly, the BH3 effector domain of PUMA Based on this, it is generally accepted that BIM and PUMA are more potent inducers of apoptosis than other BH3-only proteins such as BAD and NOXA that target only a subset of BH3-only proteins (Sinicrope et al., Clin. Cancer Res. 2008 Sep. 15; 14(18): 5810-5818). Therefore, it is hypothesized that constructs based on BAD and PUMA can efficiently induce apoptosis in cancer cells (at least to a similar degree as NOXA) once localized into cytotoxic granules by Granzyme B.

BAX is believed to be an essential protein required for Mitochondrial outer membrane permeabilization (MOMP) downstream of the pro-apoptotic pathway in target cells (Dewson et al. J. Cell. Sci. 2009 Aug. 15; 122(16): 2801-2808). Therefore, it is generally assumed that the use of BAX may lead to a faster pro-apoptotic response by directly initiating MOMP, as opposed to BH3-only proteins, which require the inhibition of BH3 pro-survival proteins. Hence, it is hypothesized that Tbax (i.e., truncated BAX) can readily induce apoptosis in cancer cells (e.g., faster than BH3-only proteins) after localization into cytotoxic granules.

WO2015157864A1 describes genetically modified cytotoxic lymphocytes to produce fusion proteins comprising granzyme B and diphtheria toxin A (DTA). It is shown in the delivery of the fusion protein to the cytosols of the target cancer cells and the target cells, the granzyme-perforin pathway of the cytotoxic lymphocytes is activated, leading to the delivery of the fusion protein to the cytosols and killing of apoptosis-resistant cancer cells. Considering that the mechanism-of-action of DTA does not involve the pro-apoptotic pathway (as for the BCL-2 family of proteins), it is hypothesized that DTA can directly and efficiently induce apoptosis in MM cancer cells, irrespective of their resistance to apoptosis.

Constructs based on NOXA are used as reference because of the already established pro-apoptotic activity, as shown in Examples 1 and 2.

Based on the previous, the following sequences are placed behind the granzyme B sequence and tested for effectiveness as pro-apoptotic constructs:
1. BIM(BIM)
2. BIM (PUMA)
3. PUMA(PUMA)
4. PUMA(BIM)
5. NOXA(NOXA)
6. Tbax
7. DTA

### 2. Methods

Constructs are produced according to the method described in Example 2, using the BIM, PUMA, NOXA, Tbax or NOXA sequences (i.e., incorporating their native or a swapped BH3 effector domain according to sequences of Table 1) placed behind the granzyme B followed by an HA-tag and a T2A sequence. For Tbax, the transmembrane domain is removed from the sequence of human BAX (final sequence according to SEQ ID NO:56) to minimize the chance that tBAX will integrate into membranes, such as the cell membrane and the membrane of the cytotoxic granules in (CAR T) lymphocytes. The fusion construct sequence for the Granzyme B-DTA construct is based on the sequence described in WO2015157864A1 (SEQ ID NO:1 in WO2015157864A1, herein incorporated by reference).

BCMA CAR T cells are transduced with the constructs and cocultured with H929 MM cells in an effector:target ratio of 1:5. Specific apoptosis is determined after 24 hours of coculture using flow cytometry as aforementioned.

The localization of the constructs into the H929 MM cells is determined by staining with fluorophore-tagged anti-HA antibody and detection by flow cytometry. The presence of fluorescence signal in the H929 MM cells is scored qualitatively in a blinded fashion.

A condition is included wherein the transduced BCMA CAR T cells are cultured alone for 24 hours and viability of the cells is determined using propidium iodide staining by flow cytometry (ThermoFisher scientific, catalog #BMS500PI), based on the manufacturer's instructions.

The results of three independent experiments are shown.

### 3. Results

As shown in Table 2, when compared to unmodified BCMA CAR T cells, the increase in specific apoptosis in H929 cells is higher with the NOXA construct than with constructs based on other pro-apoptotic proteins.

CAR T cells transduced with BIM(BIM), BIM(PUMA), PUMA(PUMA), PUMA(BIM) do not clearly show higher pro-apoptotic activity as compared to use of wildtype (i.e., untransduced) BCMA CAR T cells.

Transduction with Granzyme B-BAX constructs also does not lead to specific apoptosis, but rather is associated with reduced viability of the BCMA CAR T cells. BCMA CAR T cells transduced with the construct based on DTA show enhanced pro-apoptotic activity although to lesser extent than the NOXA construct. Furthermore, the DTA appears to strongly reduce viability of the BCMA CAR T cells. Thus, BAX and DTA may induce toxicity in the effector cells.

The Granzyme B-NOXA constructs efficiently localize into the target cells. The translocalization for the other constructs appears relatively low, considering the low fluorescence signal detected.

TABLE 2

| | Increase | | |
|---|---|---|---|
| | in specific | BCMA CAR | |
| | apoptosis of | T cell | Localization in |
| | H929 cells | viability | H929 cells |
| GzB-BIM(BIM) | <50% | 50-95% | Absent/low signal |
| GzB-BIM(PUMA) | <50% | 50-95% | Absent/low signal |
| GzB-PUMA(PUMA) | <50% | 50-95% | Absent/low signal |
| GzB-PUMA(BIM) | <50% | 50-95% | Absent/low signal |
| GzB-NOXA(NOXA) | 50-90% | 50-95% | High signal |
| GzB-BAX | <25% | <50% | Absent/low signal |
| GzB-DTA | 25-75% | <50% | Average signal |

GzB = Granzyme B, DTA = Diphteria toxin A.

### 4. Summary

The current Example shows that NOXA is the most potent pro-apoptotic protein to be used in conjunction with the granzyme-perforin trafficking system and as the backbone of the modified BH3-only proteins. Without being bound by theory, it is considered that the small size of NOXA may play a role in its potency (and the lack of potency of BIM and BAX). NOXA has the smallest size of all BCL-2 family proteins (Gross et al. Cell Death Differ. 2017. PMID: 28234359), while it remains highly functional to induce apoptosis like other BH3-only proteins. In the current Example, the constructs with BIM, PUMA, and BAX (which are relatively large proteins) appear to more poorly localize into the effector cells, despite their directed secretion into the close environment of the synapse via the granzyme-perforin pathway. The small size of NOXA as a backbone for the modified BH3-only protein also facilitates cloning, expression, and transfer to perforin pores as additional advantages.

The current Example furthermore shows that other pro-apoptotic proteins than NOXA, in particular, BAX and DTA, may negatively affect the viability of the effector T cells. Increased loss of viability is seen in effector T cells transduced with constructs with BAX and DTA. Without being bound by theory, it seems that BAX may insert itself in the membrane of the cytotoxic granules of the effector T cells, as it does in mitochondrial membranes, and/or leak into the effector T cells causing apoptosis. DTA kills primarily by inhibiting protein synthesis. It appears that the DTA may lead to toxicity in the effector cell and other cells in vicinity, such as after leakage from the (eradicated) cancer cells into the surrounding tissue.

Combined, NOXA was identified as by far the most effective option to achieve a strong pro-apoptotic effect and with minimal/no toxicity in effector or neighboring cells.

### Experimental Example 4

### 1. Background

The current example compares the efficiency of constructs using granzyme A, granzyme B, and serglycin as granule-localizing domains to deliver NOXA(BIM) in to target cells and to induce selective apoptosis.

Granzyme A, granzyme B, and serglycin are tested because they share the feature that they are naturally loaded into lytic granules upon activation of activation of T cells and NK cells.

Membrane-translocating sequences were initially also tested, but transduction of T cells with the constructs did not lead to localizing of the pro-apoptotic proteins into cytotoxic granules in the T cells, and consequently did not lead to localization into cancer cells and the induction of apoptosis. The constructs based on membrane-translocating sequences are therefore not discussed herein.

### 2. Methods

Lentiviral or retroviral constructs comprising Granzyme B and NOXA(BIM) are produced according to Example 2. NOXA(BIM) constructs are additionally designed with granzyme A (SEQ ID NO:1) or serglycin (SEQ ID NO:13) as alternative granule-localizing domain.

BCMA CAR T cells are transduced with granzyme A-NOXA(BIM), granzyme B-NOXA(BIM), and serglycin-NOXA(BIM) constructs and cocultured with H929 MM cells in an effector:target ratio of 1:5. Specific apoptosis is determined after 24 hours of coculture using flow cytometry as aforementioned.

To study the delivery of proteins comprising granzyme A, granzyme B, and seglycin into target cells, the NOXA(BIM) is replaced with the fluorescent Mscarlet and BCMA CAR T cells transduced therewith are cocultured with H929 MM cells for 16 hours and analyzed by flow cytometry. As control, untransduced BCMA CAR T cells are used.

The results of three independent experiments are shown.

### 3. Results

It is found that H929 MM cells cocultured with BCMA CAR T cells transduced with the Mscarlet TRACKER constructs based on granzyme A, granzyme B or serglycin take up Mscarlet and survive the interaction. Overall, the Mscarlet signal in the H929 MM cells is similar for the granzyme A, granzyme B, or serglycin constucts.

The specific apoptosis in H929 cells is comparable (>75%) for granzyme A-NOXA(BIM), granzyme B-NOXA(BIM) and serglycin-NOXA(BIM).

### 4. Summary

The present disclosure shows that several proteins that naturally localize into lytic granules and target cells after lymphocyte activation and can deliver NOXA and modified variants into target cells and cause selective apoptosis. In contrast, membrane-translocating sequences (i.e., which are not trafficked specifically to the cytotoxic granules) do not deliver NOXA into target cells and cause selective apoptosis.

Granzyme A, granzyme B, and serglycin share the feature that they are naturally loaded into lytic granules upon activation of activation of T cells and NK cells. Nevertheless, the findings in the present Example are surprising, because Granzyme A, granzyme B, and serglycin may follow distinct paths to trigger cellular death, but at the same time show similar efficiency in inducing apoptosis in combination with NOXA(BIM). To illustrate, granzyme A activates a caspase-independent cell death whereas Granzyme B activates apoptosis primarily by cleaving caspases and some key caspase pathway substrates (Zhu et al., Blood 114(6):1205-16). Serglycin, on the other hand, is a primary proteoglycan of cytotoxic granules is not recognized as a direct mediator of apoptosis. It was shown by others that serglycin forms complexes with mediators of the granzyme-perforin pathway and that the complexes are delivered into target cells (Metkar et al., Immunity. 2002 March; 16(3):417-28).

Without being bound by theory, it appears that granzyme A, granzyme B and serglycin share a common mechanism to deliver pro-apoptotic proteins (e.g., NOXA(BIM)) into target cells. which allows activation of the pro-apoptotic. Hence, the function of the granule-localizing domain may not be crucial for determining the efficiency of apoptosis (e.g., as also seen in Table 1 of Smyth et al., J. Leukoc. Biol. 2001 July; 70(1):18-29).

### Experimental Example 5

### 1. Background

The efficacy of the developed NOXA constructs is validated in an in vivo tumor model.

### 2. Methods

NSG mice were injected with 5×10⁶ RPMI8226-luciferase cells (human myeloma cell line). After 17 days, tumor growth was established in the mice, as determined with Bioluminescence imaging (BLI). Four days later (day 0), 8×10⁵ BCMA CAR T cells were injected with aforementioned “NOXA” construct or the inactive “Inoxa” construct. Tumor growth was measured over time with BLI. Statistical analysis was performed by mixed-effects analysis/two-way ANOVA.

### Results

There is a clear difference between the “NOXA” and “Inoxa” groups with regard to tumor growth.

It is expected that larger experimental groups will also show a (significant) difference in death (e.g., based on the Kaplan-Meier curve).

1. A nucleic acid molecule comprising:
a) a first nucleotide sequence encoding a granule-localizing domain;
b) a second nucleotide sequence encoding a pro-apoptotic protein comprising a B-cell lymphoma 2 (BCL-2) homology 3 (BH3) effector domain,
wherein the pro-apoptotic protein is:
phorbol-12-myristate-13-acetate-induced protein 1 (NOXA) and/or a pro-apoptotic protein with at least 90% sequence identity with SEQ ID NO:45; and/or
NOXA wherein the BH3 effector domain is substituted by:
a BH3 effector domain of harakiri (HRK),
a BH3 effector domain of BCL-2 associated agonist of cell death (BAD),
a BH3 effector domain of BH3-interacting domain death agonist (BID),
a BH3 effector domain of BCL-2-like protein 11 (BIM), or
a BH3 effector domain of PURbeta (PURB).

2. The nucleic acid molecule according to claim 1, wherein the granule-localizing domain is a granzyme protein or a leader peptide thereof, and/or wherein the first nucleotide sequence is:
a nucleotide sequence encoding granzyme A or a nucleotide sequence encoding a leader peptide of granzyme A;
a nucleotide sequence encoding granzyme B or a nucleotide sequence encoding a leader peptide of granzyme B;
a nucleotide sequence encoding granzyme H or a nucleotide sequence encoding a leader peptide of granzyme H;
a nucleotide sequence encoding granzyme K or a nucleotide sequence encoding a leader peptide of granzyme K;
a nucleotide sequence encoding granzyme M or a nucleotide sequence encoding a leader peptide of granzyme M;
a nucleotide sequence encoding granulysin or a nucleotide sequence encoding leader peptide of granulysin;
a nucleotide sequence encoding serglycin or a nucleotide sequence encoding a leader peptide of serglycin;
a nucleotide sequence encoding perforin or a nucleotide sequence encoding a leader peptide of perforin; and/or
a nucleotide sequence encoding an N-Acetylglucosamine-1 (GlcNAc-1) phosphotransferase binding domain.

3. The nucleic acid molecule according to claim 1, wherein:
the first nucleotide sequence is a nucleotide sequence encoding granzyme B or a nucleotide sequence encoding a leader peptide of granzyme B; and
the second nucleotide sequence encodes NOXA wherein the BH3 effector domain is substituted by a BH3 effector domain of BIM.

4. A nucleic acid molecule encoding a pro-apoptotic protein, wherein the pro-apoptotic protein is encoded by a nucleotide sequence comprising:
a) B-cell lymphoma 2 (BCL-2) homology 3 (BH3) effector domain encoding sequence;
b) an upstream domain encoding sequence, which is located upstream of the BH3 effector domain encoding sequence; and
c) a downstream domain encoding sequence, which is located downstream of the BH3 effector domain encoding sequence,
wherein the upstream domain encoding sequence encodes an upstream domain of phorbol-12-myristate-13-acetate-induced protein 1 (NOXA), and/or wherein the downstream domain encoding sequence encodes a downstream domain of NOXA,
wherein the BH3 effector domain encoding sequence encodes:
a BH3 effector domain of harakiri HRK);
a BH3 effector domain of BCL-2 associated agonist of cell death (BAD);
a BH3 effector domain of BH3 interacting domain death agonist (BID);
a BH3 effector domain of BCL-2-like protein 11 (BIM); and/or
a BH3 effector domain of PURbeta (PURB.

5. The nucleic acid molecule according to claim 4, wherein the pro-apoptotic protein is encoded by a nucleotide sequence comprising:
an upstream domain encoding sequence of NOXA;
a BH3 effector domain encoding sequence of BIM; and
a downstream domain encoding sequence of NOXA.

6. The nucleic acid molecule according to claim 4, wherein the pro-apoptotic protein is encoded by a nucleotide sequence having at least 90% sequence identity with SEQ ID NO:43.

7. The nucleic acid molecule claim 4, comprising a further nucleotide sequence that encodes a granule-localizing domain.

8. The nucleic acid molecule of claim 1, further comprising one or more of:
a) a nucleotide sequence encoding a linker molecule;
b) a nucleotide sequence encoding a cleavage site for an enzyme; or
c) a nucleotide sequence encoding a promoter operatively linked to a nucleotide sequence encoding the granule-localizing domain and/or a nucleotide sequence encoding the pro-apoptotic protein.

9. A pro-apoptotic protein comprising a BH3 effector domain, wherein the pro-apoptotic protein is encoded by a nucleic acid molecule comprising:
a) a B-cell lymphoma 2 (BCL-2) homology 3 (BH3) effector domain encoding sequence encoding a BH3 effector domain of harakiri (HRK), a BH3 effector domain of BCL-2 associated agonist of cell death (BAD), a BH3 effector domain of BH3-interacting domain death agonist (BID), a BH3 effector domain of BCL-2-like protein 11 (BIM), and/or a BH3 effector domain of PURbeta (PURB);
b) an upstream domain encoding sequence located upstream of the BH3 effector domain encoding sequence, wherein the upstream domain encoding sequence encodes an upstream domain of phorbol-12-myristate-13-acetate-induced protein 1 (NOXA); and
c) a downstream domain encoding sequence located downstream of the BH3 effector domain encoding sequence encoding a downstream domain of NOXA, and/or
has an amino acid sequence with at least 90% sequence identity with any one of SEQ ID NO:45, 46, 47, 48, 49, and 50.

10. A pro-apoptotic construct comprising:
a) a granule-localizing domain,
wherein the granule-localizing domain is granzyme A or a leader peptide of granzyme A; granzyme B or a leader peptide of granzyme B; granzyme H or a leader peptide of granzyme H; granzyme K or a leader peptide of granzyme K; granzyme M or a leader peptide of granzyme M; granulysin or a leader peptide of granulysin; serglycin or a leader peptide of serglycin; perforin or a leader peptide of perforin; and/or N-acetylglucosamine-1 (GlcNAc-1) phosphotransferase binding domain;
b) a pro-apoptotic protein,
wherein the pro-apoptotic protein is encoded by a polynucleotide comprising:
i) a B-cell lymphoma 2 (BCL-2) homology 3 (BH3) effector domain encoding sequence encoding a BH3 effector domain of harakiri (HRK), a BH3 effector domain of BCL-2 associated agonist of cell death (BAD), a BH3 effector domain of BH3-interacting domain death agonist (BID), a BH3 effector domain of BCL-2-like protein 11 (BIM), and/or a BH3 effector domain of PURbeta (PURB);
ii) an upstream domain encoding sequence located upstream of the BH3 effector domain encoding sequence, wherein the upstream domain encoding sequence encodes an upstream domain of phorbol-12-myristate-13-acetate-induced protein 1 (NOXA); and
iii) a downstream domain encoding sequence located downstream of the BH3 effector domain encoding sequence encoding a downstream domain of NOXA; and
c) a linker molecule between the granule-localizing domain and the pro-apoptotic protein.

11. The pro-apoptotic construct according to claim 10, comprising a cleavage site between the granule-localizing domain and the pro-apoptotic protein.

12. A nucleic acid delivery construct comprising the nucleic acid molecule of claim 1, wherein the nucleic acid delivery construct is one or more of a plasmid, a recombinant adenovirus, an adeno-associated virus (AAV), a retrovirus, a lentivirus, a herpes simplex virus, and a vaccinia virus.

13. A human T cell or human NK cell, wherein the human T cell or human NK cell comprises the nucleic acid molecule of claim 1.

14. A method of treating cancer the method comprising utilizing the nucleic acid molecule of claim 1 to treat the cancer.

15. The nucleic acid molecule of claim 1, wherein the pro-apoptotic protein comprises NOXA with the BH3 effector domain substituted by a BH3 effector domain of HRK encoded by a polynucleotide having at least 90% sequence identity with SEQ ID NO:19.

16. The nucleic acid molecule of claim 1, wherein the pro-apoptotic protein comprises NOXA with the BH3 effector domain substituted by a BH3 effector domain of BAD encoded by a polynucleotide having at least 90% sequence identity with SEQ ID NO:20.

17. The nucleic acid molecule of claim 1, wherein the pro-apoptotic protein comprises NOXA with the BH3 effector domain substituted by a BH3 effector domain of BID encoded by a polynucleotide having at least 90% sequence identity with SEQ ID NO:21.

18. The nucleic acid molecule of claim 1, wherein the pro-apoptotic protein comprises NOXA with the BH3 effector domain substituted by a BH3 effector domain of BIM encoded by a polynucleotide having at least 90% sequence identity with SEQ ID NO:22.

19. The nucleic acid molecule of claim 1, wherein the pro-apoptotic protein comprises NOXA with the BH3 effector domain substituted by a BH3 effector domain of PURB encoded by a polynucleotide having at least 90% sequence identity with SEQ ID NO:23.
